(19) United States
(12) Patent Application Publication
(10) Pub. No.: US 2023/0233667 A1
(43) Pub. Date: Jul. 27, 2023

(54) CORONAVIRUS VACCINE

(71) Applicant: Affinivax, Inc., Cambridge, MA (US)

(72) Inventors: Gilles R. Besin, Brookline, MA (US); Heidi Burke, Wakefield, MA (US); Richard Malley, Beverly, MA (US)

(21) Appl. No.: 17/940,967

(22) Filed: Sep. 8, 2022

Related U.S. Application Data
(60) Provisional application No. 63/241,926, filed on Sep. 8, 2021.

(51) Int. Cl. A61K 39/215

(52) U.S. Cl. CPC A61K 39/215; C07K 2319/00; A61K 2039/6087

(57) ABSTRACT

Compositions and methods for the prevention and/or treatment of SARS-CoV-2 infection and/or COVID-19 are described.

### CROSS-REFERENCE TO RELATED APPLICATIONS

The present application claims the benefit of U.S. Provisional Application No. 63/241,926 filed on Sep. 8, 2021, the contents of which are hereby incorporated herein in their entirety.

### SEQUENCE LISTING

The present application contains a Sequence Listing which has been submitted electronically in XML file format and is hereby incorporated by reference in its entirety. Said XML copy, created on Nov. 21, 2022, is named “2011588-0192_SL.xml” and is 94,168 bytes in size.

### BACKGROUND

Coronaviruses are a group of related viruses that cause diseases in mammals and birds. They are enveloped viruses with a positive-sense single-stranded RNA genome contained in a nucleocapsid. In humans, coronaviruses cause respiratory tract infections that are often mild, such as some cases of the common cold. However, certain coronaviruses can cause disease than can be severe or even lethal, such as severe acute respiratory syndrome (SARS), which is caused by the SARS coronavirus (SARS-CoV) and Middle East respiratory syndrome (MERS), a respiratory infection caused by the MERS-coronavirus (MERS-CoV). In late 2019, a new coronavirus-associated disease affecting humans emerged. The etiologic agent, named SARS-CoV-2, is related to SARS-CoV, and both of these viruses belong to a large group of virus strains collectively termed severe acute respiratory syndrome-related coronavirus (SARSr-CoV), which are known to infect non-human species such as bats. The disease caused by SARS-CoV-2 has been named coronavirus disease 2019 (COVID-19). The rapid spread of COVID-19 presents a serious threat to human health globally. While many patients experience only mild symptoms, a subset of people infected with the SARS-CoV-2 virus will progress to develop severe disease symptoms, such as acute respiratory distress syndrome, pneumonia, pulmonary edema and organ failure.

### SUMMARY

Among other things, the present disclosure provides compositions and methods for prevention and/or treatment of SARS-CoV-2 infection and COVID-19 in patient populations in need thereof.

In one aspect, the disclosure features a vaccine comprising one or more species of immunogenic complexes, wherein each immunogenic complex comprises: (a) a biotinylated polysaccharide antigen; and (b) a fusion protein comprising: (i) a biotin-binding moiety; and (ii) at least one polypeptide antigen of SARS-CoV-2; wherein the biotinylated polysaccharide antigen is non-covalently associated with the biotin-binding moiety of the fusion protein to form an immunogenic complex.

In some embodiments, the fusion protein comprises at least one of: (a) a Spike (S) polypeptide antigen or antigenic fragment thereof, (b) an Envelope (E) polypeptide antigen or antigenic fragment thereof; (c) a Membrane (M) polypeptide antigen or antigenic fragment thereof; and (d) a Nucleocapsid (N) polypeptide or antigenic fragment thereof.

In some embodiments, the fusion protein comprises at least one of: (a) a Spike (S) polypeptide antigen or antigenic fragment thereof, and (b) a Membrane (M) polypeptide antigen or antigenic fragment thereof.

In some embodiments, the fusion protein comprises the Receptor Binding Domain (RBD) of a Spike (S) polypeptide antigen or antigenic fragment thereof. In some embodiments, the fusion protein comprises one or more Extra-Cellular Domains (ECDs) of a Membrane (M) polypeptide antigen or antigenic fragment thereof. In some embodiments, the fusion protein comprises: (a) the Receptor Binding Domain (RBD) of a Spike (S) polypeptide antigen or antigenic fragment thereof; and (b) one or more Extra-Cellular Domains (ECDs) of a Membrane (M) polypeptide antigen or antigenic fragment thereof.

In some embodiments, the one or more species of immunogenic complexes comprise polypeptide antigen(s) of one strain (variant) of SARS-CoV-2. In some embodiments, the one or more species of immunogenic complexes comprise polypeptide antigen(s) of multiple strains (variants) of SARS-CoV-2.

In some embodiments, the vaccine comprises one species of immunogenic complexes, wherein the species comprises the same fusion protein. In some embodiments, the vaccine comprises a plurality of different species of immunogenic complexes, wherein the plurality of different species comprises a plurality of different fusion proteins.

In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, 18, 21, 24, 27, 30, 33, 36, 39 or 42, or an antigenic fragment thereof. In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, or 18, or an antigenic fragment thereof. In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:33, 36, 39 or 42, or an antigenic fragment thereof.

In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, 20, 23, 26, 29, 32, 35, 38, 41 or 44, or an antigenic fragment thereof. In some embodiments, the fusion protein is or comprises the amino acid sequence of SEQ ID NO:5, 8, 11, 14, 17, 20, 23, 26, 29, 32, 35, 38, 41 or 44, or an antigenic fragment thereof. In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, or 20, or an antigenic fragment thereof. In some embodiments, the fusion protein is or comprises the amino acid sequence of SEQ ID NO:5, 8, 11, 14, 17, or 20, or an antigenic fragment thereof. In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:35, 38, 41 or 44, or an antigenic fragment thereof. In some embodiments, the fusion protein is or comprises the amino acid sequence of SEQ ID NO:35, 38, 41 or 44, or an antigenic fragment thereof.

In some embodiments, the biotinylated polysaccharide antigen comprises a polysaccharide of Streptococcus pneumoniae. In some embodiments, the biotinylated polysaccharide antigen comprises a polysaccharide of Streptococcus pneumoniae selected from serotypes 1, 9N, and 19A. In some embodiments, the biotinylated polysaccharide antigen comprises a polysaccharide of Streptococcus pneumoniae serotype 1 (PS1).

In some embodiments, the biotin-binding moiety is a polypeptide comprising (i) an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:1 or a biotin-binding fragment thereof; or (ii) a polypeptide comprising an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:2, or a biotin-binding fragment thereof.

In another aspect, the disclosure features an immunogenic complex comprising: (a) a biotinylated polysaccharide antigen; and (b) a fusion protein comprising: (i) a biotin-binding moiety; and (ii) at least one polypeptide antigen of SARS-CoV-2; wherein the biotinylated polysaccharide antigen is non-covalently associated with the biotin-binding moiety of the fusion protein.

In some embodiments, the fusion protein comprises at least one of: (a) a Spike (S) polypeptide antigen or antigenic fragment thereof, (b) an Envelope (E) polypeptide antigen or antigenic fragment thereof; (c) a Membrane (M) polypeptide antigen or antigenic fragment thereof; and (d) a Nucleocapsid (N) polypeptide or antigenic fragment thereof. In some embodiments, the fusion protein comprises at least one of: (a) a Spike (S) polypeptide antigen or antigenic fragment thereof; and (b) a Membrane (M) polypeptide antigen or antigenic fragment thereof. In some embodiments, the fusion protein comprises the Receptor Binding Domain (RBD) of a Spike (S) polypeptide antigen or antigenic fragment thereof. In some embodiments, the fusion protein comprises one or more Extra-Cellular Domains (ECDs) of a Membrane (M) polypeptide antigen or antigenic fragment thereof. In some embodiments, the fusion protein comprises: (a) the Receptor Binding Domain (RBD) of a Spike (S) polypeptide antigen or antigenic fragment thereof; and (b) one or more Extra-Cellular Domains (ECDs) of a Membrane (M) polypeptide antigen or antigenic fragment thereof.

In some embodiments, the immunogenic complex comprises one polypeptide antigen. In some embodiments, the immunogenic complex comprises more than one polypeptide antigen. In some embodiments, the immunogenic complex comprises polypeptide antigen(s) of one strain (variant) of SARS-CoV-2. In some embodiments, the immunogenic complex comprises polypeptide antigen(s) of multiple strains (variants) of SARS-CoV-2.

In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, 18, 21, 24, 27, 30, 33, 36, 39 or 42, or an antigenic fragment thereof. In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, or 18, an antigenic fragment thereof. In some embodiments, the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:33, 36, 39 or 42, or an antigenic fragment thereof.

In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5, 8, 11, 14, 17, 20, 23, 26, 29, 32, 35, 38, 41 or 44, or an antigenic fragment thereof. In some embodiments, the fusion protein is or comprises the amino acid sequence of SEQ ID NO:5, 8, 11, 14, 17, 20, 23, 26, 29, 32, 35, 38, 41 or 44, or an antigenic fragment thereof. In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 5, 8, 11, 14, 17, 20, or an antigenic fragment thereof. In some embodiments, the fusion protein is or comprises the amino acid sequence of SEQ ID NO: 5, 8, 11, 14, 17, 20, or an antigenic fragment thereof. In some embodiments, the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 35, 38, 41 or 44, or an antigenic fragment thereof. In some embodiments, the fusion protein is or comprises the amino acid sequence of SEQ ID NO: 35, 38, 41 or 44, or an antigenic fragment thereof.

In some embodiments, the biotinylated polysaccharide antigen comprises a polysaccharide of Streptococcus pneumoniae. In some embodiments, the biotinylated polysaccharide antigen comprises a polysaccharide of Streptococcus pneumoniae selected from serotypes 1, 9N, and 19A. In some embodiments, the biotinylated polysaccharide antigen comprises a polysaccharide of Streptococcus pneumoniae serotype 1 (PS1).

In some embodiments, the biotin-binding moiety is or comprises a rhizavidin polypeptide. In some embodiments, the biotin-binding moiety is a polypeptide comprising (i) an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:1 or a biotin-binding fragment thereof, or (ii) a polypeptide comprising an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:2, or a biotin-binding fragment thereof. In some embodiments, the polypeptide of (i) and/or (ii) comprises one or more mutations.

In another aspect, the disclosure features a vaccine comprising one or more of the immunogenic complexes disclosed herein.

In another aspect, the disclosure features a pharmaceutical composition comprising any of the vaccines described herein, and a pharmaceutically acceptable carrier. In another aspect, the disclosure features a pharmaceutical composition comprising an immunogenic complex disclosed herein, and a pharmaceutically acceptable carrier.

In some embodiments, the pharmaceutical composition further comprises one or more adjuvants. In some embodiments, the one or more adjuvants are or comprise a co-stimulation factor. In some embodiments, the one or more adjuvants are selected from the group consisting of aluminum phosphate, aluminum hydroxide, and phosphated aluminum hydroxide. In some embodiments, the one or more adjuvants are or comprise aluminum phosphate.

In some embodiments, the pharmaceutical composition is formulated for injection.

In some embodiments, upon administration to a subject, the pharmaceutical composition induces an immune response. In some embodiments, the immune response comprises an antibody and/or B cell response. In some embodiments, the antibody and/or B cell response comprises a memory B cell response. In some embodiments, the immune response comprises a T cell response. In some embodiments, the immune response comprises a CD4+ T cell response (e.g., T_{H}1, T_{H}2, or T_{H}17 response); a CD8+ T cell response; a CD4+ and CD8+ T cell response; or a CD4−/CD8− T cell response. In some embodiments, the T cell response comprises a memory T cell response. In some embodiments, the immune response comprises (i) an antibody or B cell response and (ii) a T cell response. In some embodiments, the immune response is to (i) at least one polysaccharide antigen of the vaccine or immunogenic complex, and/or (ii) at least one polypeptide antigen of the vaccine or immunogenic complex. In some embodiments, the immune response comprises (i) an antibody or B cell response to at least one polysaccharide antigen of the vaccine or immunogenic complex, and (ii) a T cell response to at least one polypeptide antigen of the vaccine or immunogenic complex. In some embodiments, the immune response comprises (i) an antibody or B cell response to at least one polysaccharide antigen of the vaccine or immunogenic complex, and (ii) a CD4+ T cell response (e.g., T_{H}1, T_{H}2, or T_{H}17 response), a CD8+ T cell response, a CD4+ and CD8+ T cell response, or a CD4−/CD8− T cell response to at least one polypeptide antigen of the vaccine or immunogenic complex. In some embodiments, the immune response comprises (i) an antibody or B cell response to at least one polysaccharide antigen of the vaccine or immunogenic complex, and (ii) an antibody or B cell response to at least one polypeptide antigen of the vaccine or immunogenic complex. In some embodiments, the immune response comprises (i) an antibody or B cell response to at least one polysaccharide antigen of the vaccine or immunogenic complex, and (ii) an antibody or B cell response; and a CD4+ T cell response (including T_{H}1, T_{H}2, or T_{H}17 response), a CD8+ T cell response, a CD4+ and CD8+ T cell response, or a CD4−/CD8− T cell response to at least one polypeptide antigen of the vaccine or immunogenic complex.

In some embodiments, upon administration to a subject, the pharmaceutical composition induces neutralizing antibodies against one or more strains (variants) of SARS-CoV-2. In some embodiments, upon administration to a subject, the pharmaceutical composition reduces or inhibits transmission of one or more strains (variants) of SARS-CoV-2 from the subject to another subject. In some embodiments, upon administration to a subject, the pharmaceutical composition reduces or inhibits replication, and/or reduces viral load, of one or more strains (variants) of SARS-CoV-2. In some embodiments, upon administration to a subject, the pharmaceutical composition inhibits, or reduces the rate of occurrence of, COVID-19 associated with or induced by one or more strains (variants) of SARS-CoV-2. In some embodiments, upon administration to a subject, the pharmaceutical composition reduces the severity of COVID-19 associated with or induced by one or more strains (variants) of SARS-CoV-2. In some embodiments, upon administration to a subject, the pharmaceutical composition inhibits, or reduces the rate of occurrence of, pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2. In some embodiments, upon administration to a subject, the pharmaceutical composition reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2. In some embodiments, upon administration to a subject, the pharmaceutical composition inhibits, or reduces the rate of, shedding of one or more strains (variants) of SARS-CoV-2. In some embodiments, upon administration to a subject, the pharmaceutical composition inhibits, or reduces the rate of, asymptomatic infection by one or more strains (variants) of SARS-CoV-2.

In another aspect, the disclosure features a method of making a vaccine, comprising non-covalently complexing a plurality of biotinylated polysaccharide antigens with a plurality of fusion proteins, wherein each fusion protein comprises at least one polypeptide antigen of SARS-CoV-2 selected from: (a) a Spike (S) polypeptide antigen or antigenic fragment thereof; (b) an Envelope (E) polypeptide antigen or antigenic fragment thereof, (c) a Membrane (M) polypeptide antigen or antigenic fragment thereof; and (d) a Nucleocapsid (N) polypeptide or antigenic fragment thereof.

In some embodiments, the plurality of biotinylated polysaccharide antigens comprises polysaccharides of Streptococcus pneumoniae serotype 1.

In another aspect, the disclosure features a method of immunizing a subject against one or more strains (variants) of SARS-CoV-2 comprising administering to the subject an immunologically effective amount of any of the vaccines described herein. In another aspect, the disclosure features a method of immunizing a subject against one or more strains (variants) of SARS-CoV-2 comprising administering to the subject an immunologically effective amount of any of the immunogenic complexes described herein. In another aspect, the disclosure features a method of immunizing a subject against one or more strains (variants) of SARS-CoV-2 comprising administering to the subject an immunologically effective amount of any of the pharmaceutical compositions described herein.

In some embodiments, the vaccine, immunogenic composition, or pharmaceutical composition induces an immune response. In some embodiments, the immune response comprises an antibody or B cell response. In some embodiments, the antibody and/or B cell response comprises a memory B cell response. In some embodiments, the immune response comprises a T cell response. In some embodiments, the immune response comprises a CD4+ T cell response (e.g., T_{H}1, T_{H}2, or T_{H}17 response), a CD8+ T cell response, a CD4+ and CD8+ T cell response, or a CD4−/CD8− T cell response. In some embodiments, the T cell response comprises a memory T cell response. In some embodiments, the immune response comprises (i) an antibody or B cell response, and (ii) a T cell response. In some embodiments, the immune response comprises (i) an antibody or B cell response, and (ii) a CD4+ T cell response (e.g., T_{H}1, T_{H}2, or T_{H}17 response), a CD8+ T cell response, a CD4+ and CD8+ T cell response, or a CD4-/CD8− T cell response. In some embodiments, the immune response is to at least one polypeptide of a fusion protein. In some embodiments, the vaccine induces neutralizing antibodies against one or more strains (variants) of SARS-CoV-2. In some embodiments, the vaccine reduces or inhibits transmission of one or more strains (variants) of SARS-CoV-2 from the subject to another subject. In some embodiments, the vaccine reduces or inhibits replication, and/or reduces viral load, of one or more strains (variants) of SARS-CoV-2. In some embodiments, the vaccine inhibits, or reduces the rate of occurrence of, COVID-19 associated with or induced by one or more strains (variants) of SARS-CoV-2. In some embodiments, the vaccine reduces the severity of COVID-19 associated with or induced by one or more strains (variants) of SARS-CoV-2. In some embodiments, the vaccine inhibits, or reduces the rate of occurrence of, pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2. In some embodiments, the vaccine reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2. In some embodiments, the vaccine inhibits, or reduces the rate of, shedding of one or more strains (variants) of SARS-CoV-2. In some embodiments, the vaccine inhibits, or reduces the rate of, asymptomatic infection by one or more strains (variants) of SARS-CoV-2.

In some embodiments, the subject is immunized against one or more strains (variants) of SARS-CoV-2 with one dose of the vaccine. In some embodiments, the subject is immunized against one or more strains (variants) of SARS-CoV-2 with two doses of the vaccine (e.g., two doses of the same vaccine, or a first dose of a first vaccine and a second dose of a second vaccine). In some embodiments, the subject is immunized against one or more strains (variants) of SARS-CoV-2 with three doses of the vaccine (e.g., three doses of the same vaccine, or three doses comprising at least two different vaccines). In some embodiments, the subject is immunized against one or more strains (variants) of SARS-CoV-2 with periodic doses of the vaccine (e.g., doses of the same vaccine or doses comprising at least two different vaccines). In some embodiments, the subject is immunized against one or more strains (variants) of SARS-CoV-2 with annual doses of the vaccine (e.g., doses of the same vaccine or doses comprising at least two different vaccines).

In some embodiments, the vaccine is administered in a regimen as a priming vaccine. In some embodiments, the vaccine is administered in a regimen as a booster vaccine. In some embodiments, the vaccine is administered in a regimen as a priming vaccine and a booster vaccine. In some embodiments, the regimen comprises administration of one or more additional vaccines.

In another aspect, the disclosure features a fusion protein comprising: (i) a biotin-binding moiety; (ii) at least one polypeptide antigen of SARS-CoV-2.

In some embodiments, the at least one polypeptide antigen of SARS-CoV-2 is selected from: (a) a Spike (S) polypeptide antigen or antigenic fragment thereof, (b) an Envelope (E) polypeptide antigen or antigenic fragment thereof; (c) a Membrane (M) polypeptide antigen or antigenic fragment thereof, and (d) a Nucleocapsid (N) polypeptide or antigenic fragment thereof. In some embodiments, the at least one polypeptide antigen of SARS-CoV-2 is selected from: (a) a Spike (S) polypeptide antigen or antigenic fragment thereof; and (b) a Membrane (M) polypeptide antigen or antigenic fragment thereof.

In some embodiments, the fusion protein comprises: (i) a biotin-binding moiety comprising an amino acid sequence at least 80%, at least 85%, at least 90%, at least 95%, at least 98%, at least 99%, or 100% identical to SEQ ID NO:2 or a biotin binding portion thereof, and (ii) a polypeptide antigen comprising an amino acid sequence at least 80%, at least 85%, at least 90%, at least 95%, at least 98%, at least 99%, or 100% identical to any of SEQ ID NO:3, 6, 9, 12, 15, 18, 21, 24, 27, 30, 33, 36, 39 or 42, or an antigenic fragment thereof. In some embodiments, the fusion protein comprises: (i) a biotin-binding moiety comprising an amino acid sequence at least 80%, at least 85%, at least 90%, at least 95%, at least 98%, at least 99%, or 100% identical to SEQ ID NO:2 or a biotin binding portion thereof, and (ii) a polypeptide antigen comprising an amino acid sequence at least 80%, at least 85%, at least 90%, at least 95%, at least 98%, at least 99%, or 100% identical to any of SEQ ID NO:3, 6, 9, 12, 15, or 18, or an antigenic fragment thereof. In some embodiments, the fusion protein comprises: (i) a biotin-binding moiety comprising an amino acid sequence at least 80%, at least 85%, at least 90%, at least 95%, at least 98%, at least 99%, or 100% identical to SEQ ID NO:2 or a biotin binding portion thereof; and (ii) a polypeptide antigen comprising an amino acid sequence at least 80%, at least 85%, at least 90%, at least 95%, at least 98%, at least 99%, or 100% identical to any of SEQ ID NO:33, 36, 39 or 42, or an antigenic fragment thereof. In some embodiments, the biotin-binding moiety of any of the foregoing fusion proteins comprises one or more mutations.

In some embodiments, the biotin-binding moiety is C-terminally linked at a polypeptide antigen. In some embodiments, the biotin-binding moiety is N-terminally linked to a polypeptide antigen.

In another aspect, the disclosure features a fusion protein comprising an amino acid sequence at least 80%, at least 85%, at least 90%, at least 95%, at least 96%, at least 97%, at least 98%, at least 99%, or at least 99.5% identical to any of SEQ ID NO: 5, 8, 11, 14, 17, 20, 23, 26, 29, 32, 35, 38, 41 or 44, or an antigenic fragment thereof.

In another aspect, the disclosure features a fusion protein comprising an amino acid sequence at least 80%, at least 85%, at least 90%, at least 95%, at least 96%, at least 97%, at least 98%, at least 99%, or at least 99.5% identical to any of SEQ ID NO: 5, 8, 11, 14, 17, or 20, or an antigenic fragment thereof.

In another aspect, the disclosure features a fusion protein comprising an amino acid sequence at least 80%, at least 85%, at least 90%, at least 95%, at least 96%, at least 97%, at least 98%, at least 99%, or at least 99.5% identical to any of SEQ ID NO: 35, 38, 41 or 44, or an antigenic fragment thereof.

In another aspect, the disclosure features a fusion protein comprising the amino acid sequence of any of SEQ ID NO: 5, 8, 11, 14, 17, 20, 23, 26, 29, 32, 35, 38, 41 or 44, or an antigenic fragment thereof.

In another aspect, the disclosure features a fusion protein comprising the amino acid sequence of any of SEQ ID NO: 5, 8, 11, 14, 17, or 20, or an antigenic fragment thereof.

In another aspect, the disclosure features a fusion protein comprising the amino acid sequence of any of SEQ ID NO: 35, 38, 41 or 44, or an antigenic fragment thereof.

In another aspect, the disclosure features a nucleic acid that comprises a nucleotide sequence encoding any of the fusion proteins described herein.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 is a schematic representation of an exemplary Multiple Antigen Presenting System (MAPS). In the exemplary embodiment shown, MAPS immunogenic complexes comprise (i) fusion proteins of the biotin-binding protein rhizavidin and proteins of interest, and (ii) a biotinylated polysaccharide. In this figure, a MAPS complex is formed between one or more fusion proteins and a biotinylated polysaccharide by non-covalent binding of rhizavidin to biotin.

FIG. 2 is a schematic of two exemplary fusion proteins that e.g., can be used in a MAPS complex.

FIGS. 3A-3F show results of a non-human primate (cynomolgus macaque) study of an exemplary MAPS SARS-CoV-2 vaccine. FIG. 3A is a schematic of the study. FIGS. 3B, 3C, and 3D depict total and specific antibody responses against SARS-CoV-2 S-RBD and other targets following immunization with saline (placebo) or exemplary vaccine. Antibody responses were measured at baseline (Day 0 of the study), 21 days post-first injection (Day 21 of the study, before the second injection; P1) and again 21 days post-second injection (Day 42 of the study; P2) by ELISA. Each point on the graphs represents results for one animal. FIG. 3B: Total IgG levels (μg/mL) against SARS-CoV-2 S-RBD in Day 0, Day 21, and Day 42 sera of saline- and exemplary vaccine-immunized animals. FIG. 3C: Subclass IgG1 levels (μg/mL), respectively, against SARS-CoV-2 Spike protein (S), S1 subunit, S2 subunit, S-RBD, Nucleoprotein (N), and unrelated influenza HA protein in Day 42 sera of saline- and exemplary vaccine-immunized animals. P=saline (open circles); V=vaccine (filled circles). FIG. 3D: Subclass IgG3 levels (μg/mL) against SARS-CoV-2 Spike protein (S), S1 subunit, S2 subunit, S-RBD, Nucleoprotein (N), and unrelated influenza HA protein in Day 42 sera of saline- and exemplary vaccine-immunized animals. P=saline (open circles); V=vaccine (filled circles). FIG. 3E depicts SARS-CoV-2 virus neutralization titers (IC50) following immunization with saline or exemplary vaccine. Neutralization titers were evaluated in Day 0, Day 21, Day 42, and Day 49 sera of saline- and exemplary vaccine-immunized animals. FIG. 3F shows cross-reactive antibody responses against S-RBD of different SARS-CoV-2 strains. The graph depicts IgG levels (μg/mL) against S-RBD of strains D614G (ancestral Wuhan), B.1.1.7 (UK) or B.1.351 (South Africa) in Day 42 sera of exemplary vaccine-immunized animals (black circles) and sera collected from seroconverted human patients (gray circles).

FIGS. 4A-4E show antibody effector function and Fc receptor binding following immunization of non-human primates with exemplary vaccine. Antibody-dependent neutrophil phagocytosis (ADNP, FIG. 4A); antibody-dependent cellular phagocytosis (ADCP, FIG. 4B); and antibody-dependent complement deposition (ADCD, FIG. 4C) was analyzed using the SARS-CoV-2 Spike protein (S) or Nucleoprotein (N) in Day 42 sera of saline- or vaccine-immunized animals. FIGS. 4D and 4E show binding of SARS-CoV-2 specific-antibodies to Fcγ receptor 2A (FcγR2A; FIG. 4D) and Fcγ receptor 3A (FcγR3A; FIG. 4E) in the presence of SARS-CoV-2 Spike protein (S), S1 subunit, S2 subunit, S-RBD, Nucleoprotein (N), and unrelated influenza HA protein, in Day 42 sera of saline- and exemplary vaccine-immunized animals. In FIGS. 4D and 4E, P=saline (open circles); V=vaccine (filled circles).

FIG. 5A shows the presence of IFN-γ secreting cells following immunization of non-human primates with exemplary vaccine. FIG. 5B shows the presence of IL-17 secreting cells following immunization of non-human primates with exemplary vaccine.

FIG. 6 shows induction of CD4+ and CD8+ T cell responses in non-human primates following two doses of exemplary vaccine.

FIGS. 7A-7B show efficacy of exemplary vaccine against nasopharyngeal viral replication and active viral shedding in non-human primates. FIG. 7A shows viral replication assessed by the Tissue Culture Infectious Dose (TCID₅₀) assay on nasal swabs collected on each of days 1-7 after challenge. FIG. 7B shows viral replication assessed by analysis of SARS-CoV-2 subgenomic RNA (sgRNA) on nasal swabs collected on days 2, 3, 4, 6 and 8 after challenge.

FIGS. 8A-8B show efficacy of exemplary vaccine against pulmonary infection in non-human primates. FIG. 8A shows viral replication assessed by the TCID₅₀ assay on BAL collected on the indicated days. FIG. 8B shows viral replication assessed by analysis of SARS-CoV-2 subgenomic RNA (sgRNA) on BAL collected on the indicated days.

FIG. 9A shows total IgG levels in μg/mL against SARS-CoV-2 Spike protein (S) in P0 (Day 0), P1 (Day 2), and P2 (Day 42) sera of saline- or exemplary vaccine-immunized rabbits. FIG. 9B shows IC50 neutralizing antibody titers against SARS-CoV-2 Spike protein (S) in P0 (Day 0), P1 (Day 2), and P2 (Day 42) sera of saline- or exemplary vaccine-immunized rabbits. Open circles=saline; black diamonds=vaccine.

FIGS. 10A and 10B depict representative variants of concern (VOCs). FIG. 10B is reproduced from Vo et al., Microorganisms, 10(3), 598 (2022).

FIG. 11 is a schematic of an exemplary multivariant MAPS vaccine (Multivariant MAPS Vaccine #1) comprising a mixture of three monovariant MAPS vaccines.

FIG. 12A shows total IgG levels in μg/mL against S-RBD of the strains indicated on the x axis, in Day 42 sera of rabbits immunized with either saline or exemplary Multivariant MAPS Vaccine #1. FIG. 12B shows IC50 neutralizing antibody titers against S-RBD of the strains indicated on the x axis, in Day 42 sera of rabbits immunized with either saline or exemplary Multivariant MAPS Vaccine #1.

FIGS. 13A-13C show total IgG levels in μg/mL against SRBD of the ancestral Wuhan D614G (FIG. 13A), B.1.1.7 (UK, Alpha variant) (FIG. 13B), and B.1.351 (South Africa, Beta variant) (FIG. 13C) strains, following immunization with the indicated monovariant MAPS vaccines or exemplary Multivariant MAPS Vaccine #1 shown schematically in FIG. 11.

FIG. 14 shows IC50 neutralizing antibody titer against S-RBD of the ancestral Wuhan D614G, B.1.1.7 (UK, Alpha variant), and B.1.351 (South Africa, Beta variant) strains, following immunization with the indicated monovariant MAPS vaccines or exemplary Multivariant MAPS Vaccine #1 shown schematically in FIG. 11.

FIG. 15 shows the design of adoptive transfer Study 1.

FIG. 16 shows the design of adoptive transfer Study 2.

FIG. 17A and FIG. 17B together form a table showing exemplary structures of S. pneumoniae antigenic polysaccharides of serotypes 1, 9N, and 19A.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastrical, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: In its broadest sense, the term “amino acid”, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Non-standard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]).

Antigen: The term “antigen”, as used herein, refers to (i) an agent that induces an immune response; and/or (ii) an agent that binds to a T cell receptor (e.g., when presented by an MHC molecule) or to an antibody. In some embodiments, an antigen induces a humoral response (e.g., including production of antigen-specific antibodies); in some embodiments, an antigen induces a cellular response (e.g., involving T cells whose receptors specifically interact with the antigen). In some embodiments, an antigen induces a humoral response and a cellular response. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)), etc. In some embodiments, an antigen is or comprises a polypeptide. In some embodiments, an antigen is or comprises a polysaccharide. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen is a polypeptide or a polysaccharide that, upon administration to a subject, induces a specific and/or clinically relevant immune response to such polypeptide or polysaccharide. In some embodiments, an antigen is selected to induce a specific and/or clinically relevant immune response to such polypeptide or polysaccharide.

Associated with: Two entities are “associated” with one another, as that term is used herein, if the presence, level and/or form of one is correlated with that of the other. In some embodiments, two or more entities are physically “associated” with one another if they interact, directly or indirectly, so that they are and/or remain in physical proximity with one another. In some embodiments, two or more entities that are physically associated with one another are covalently linked to one another. In some embodiments, two or more entities that are physically associated with one another are not covalently linked to one another but are non-covalently associated, for example by means of affinity interactions, electrostatic interactions, hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Carrier protein: As used herein, the term “carrier protein” refers to a protein or peptide that is coupled, or complexed, or otherwise associated with a hapten (e.g., a small peptide or lipid) or less immunogenic antigen (e.g., a polysaccharide) and that induces or improves an immune response to such a coupled, or complexed, or otherwise associated hapten (e.g., a small peptide or lipid) or less immunogenic antigen (e.g., a polysaccharide). In some embodiments, such an immune response is or comprises a response to a hapten or less immunogenic antigen that is coupled, or complexed, or otherwise associated with such a carrier protein. In some embodiments, such an immune response is or comprises a response to both a carrier protein and a hapten or less immunogenic antigen that is coupled, or complexed, or otherwise associated with such a carrier protein. In some embodiments, no significant immune response to a carrier protein itself occurs. In some embodiments, immune response to a carrier protein may be detected; in some embodiments, immune response to such a carrier protein is strong. In some embodiments, a carrier protein is coupled, or complexed, or otherwise associated with one or more other molecules.

Combination therapy: As used herein, the term “combination therapy” refers to those situations in which a subject is exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Derivative: As used herein, the term “derivative”, or grammatical equivalents thereof, refers to a structural analogue of a reference substance. That is, a “derivative” is a substance that shows significant structural similarity with the reference substance, for example sharing a core or consensus structure, but also differs in certain discrete ways. Such a substance would be said to be “derived from” said reference substance. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Fragment: A “fragment” of a material or entity as described herein has a structure that includes a discrete portion of the whole, but lacks one or more moieties found in the whole. In some embodiments, a fragment consists of such a discrete portion. In some embodiments, a fragment includes a discrete portion of the whole which discrete portion shares one or more functional characteristics found in the whole. In some embodiments, a fragment consists of such a discrete portion. In some embodiments, a fragment consists of or comprises a characteristic structural element or moiety found in the whole. In some embodiments, a fragment of a polymer, e.g., a polypeptide or a polysaccharide, comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improve, increase, inhibit or reduce: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Immunologically effective amount or immunologically effective dose: As used herein, “immunologically effective amount” or “immunologically effective dose” refers to an amount of an antigenic or immunogenic substance, e.g., an antigen, immunogen, immunogenic complex, immunogenic composition, vaccine, or pharmaceutical composition, which when administered to a subject, either in a single dose or as part of a series of doses, that is sufficient to enhance a subject's own immune response against a subsequent exposure to a pathogen. An immunologically effective amount may vary based on the subject to be treated, the species of the subject, the degree of immune response desired to induce, etc. In some embodiments, an immunologically effective amount is sufficient for treatment or protection of a subject having or at risk of having disease. In some embodiments, an immunologically effective amount refers to a non-toxic but sufficient amount that can be an amount to treat, attenuate, or prevent infection and/or disease (e.g., a sign or symptom associated with infection and/or disease) in any subject. In some embodiments, an immunologically effective amount is sufficient to induce an immunoprotective response upon administration to a subject.

Immunoprotective response or protective response: As used herein, “immunoprotective response” or “protective response” refers to an immune response that mediates antigen or immunogen-induced immunological memory. In some embodiments, an immunoprotective response is induced by the administration of a substance, e.g., an antigen, immunogen, immunogenic complex, immunogenic composition, vaccine, or pharmaceutical composition to a subject. In some embodiments, immunoprotection involves one or more of active immune surveillance, a more rapid and effective response upon immune activation as compared to a response observed in a naïve subject, efficient clearance of the activating agent or pathogen, followed by rapid resolution of inflammation. In some embodiments, an immunoprotective response is an adaptive immune response. In some embodiments, an immunoprotective response is sufficient to protect an immunized subject from productive infection by a particular pathogen or pathogens to which a vaccine is directed (e.g., SARS-CoV-2 nfection).

Immunization: As used herein, “immunization”, or grammatical equivalents thereof, refers to a process of inducing an immune response to an infectious organism or agent in a subject (“active immunization”), or alternatively, providing immune system components against an infectious organism or agent to a subject (“passive immunization”). In some embodiments, immunization involves the administration of one or more antigens, immunogens, immunogenic complexes, vaccines, immune molecules such as antibodies, immune sera, immune cells such as T cells or B cells, or pharmaceutical compositions to a subject. In some embodiments, immunization is performed by administering an immunologically effective amount of a substance, e.g., an antigen, immunogen, immunogenic complex, immunogenic composition, vaccine, immune molecule such as an antibody, immune serum, immune cell such as a T cell or B cell, or pharmaceutical composition to a subject. In some embodiments, immunization results in an immunoprotective response in the subject. In some embodiments, active immunization is performed by administering to a subject an antigenic or immunogenic substance, e.g., an antigen, immunogen, immunogenic complex, vaccine, or pharmaceutical composition. In some embodiments, passive immunization is performed by administering to a subject an immune system component, e.g., an immune molecule such as an antibody, immune serum, or immune cell such as a T cell or B cell.

Isolated: As used herein, the term “isolated”, or grammatical equivalents thereof, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polysaccharide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide or polysaccharide that is chemically synthesized or is synthesized in a cellular system different from that which produces it in nature is considered to be an “isolated” polypeptide or polysaccharide. Alternatively or additionally, in some embodiments, a polypeptide or polysaccharide that has been subjected to one or more purification techniques may be considered to be an “isolated” polypeptide or polysaccharide to the extent that it has been separated from other components a) with which it is associated in nature; and/or b) with which it was associated when initially produced.

Linker: As used herein, the term “linker” is used to refer to an entity that connects two or more elements to form a multi-element agent. For example, those of ordinary skill in the art appreciate that a polypeptide whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, a polypeptide linker is at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids in length. In some embodiments, a linker is characterized in that it tends not to adopt a rigid three-dimensional structure, but rather provides flexibility to the polypeptide. A variety of different linker elements that can appropriately be used when engineering polypeptides (e.g., fusion polypeptides) are known in the art (Holliger et al, 1993; Poljak, 1994).

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Polysaccharide: The term “polysaccharide” as used herein refers to a polymeric carbohydrate molecule composed of long chains of monosaccharide units bound together by glycosidic, phosphodiester, or other linkages and on hydrolysis give the constituent monosaccharides or oligosaccharides. Polysaccharides range in structure from linear to highly branched. Examples include storage polysaccharides such as starch and glycogen, structural polysaccharides such as cellulose and chitin and microbial polysaccharides, and antigenic polysaccharides found in microorganisms including, but not limited to, capsular polysaccharides (CPS), O polysaccharides (OPS), core O polysaccharides (COPS), and lipopolysaccharides (LPS).

Polypeptide: The term “polypeptide”, as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Prevention: The term “prevent” or “prevention”, as used herein in connection with a disease, disorder, and/or medical condition, refers to reducing the risk of developing the disease, disorder and/or condition, and/or a delay of onset, and/or reduction in frequency and/or severity of one or more characteristics or symptoms of a particular disease, disorder or condition. In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition. In some embodiments, prevention may be considered complete when onset of a disease, disorder or condition has been delayed for a pre-defined period of time.

Protein: As used herein, the term “protein” encompasses a polypeptide. Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: As used herein, the term “recombinant” is intended to refer to polypeptides that are designed, engineered, prepared, expressed, created, manufactured, and/or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, subject, population, sample, sequence or value of interest is compared with a reference or control agent, animal, subject, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Response: As used herein, a “response” to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. Subject response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of biomarkers in a sample obtained from a subject, cytology, and/or histology. The exact response criteria can be selected in any appropriate manner, provided that when comparing groups of subjects and/or tumors, the groups to be compared are assessed based on the same or comparable criteria for determining response rate. One of ordinary skill in the art will be able to select appropriate criteria.

Risk: As will be understood from context, “risk” of a disease, disorder, and/or condition refers to a likelihood that a particular subject will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from subjects comparable to a particular subject. In some embodiments, relative risk is 0, 1, 2, 3, 4, 5, 6, 7, 8, 9, 10, or more.

Serotype: As used herein, the term “serotype”, also referred to as a serovar, refers to a distinct variation within a species of bacteria or virus or among immune cells of different subjects. These microorganisms, viruses, or cells are classified together based on their cell surface antigens, allowing the epidemiologic classification of organisms to the sub-species level. A group of serovars with common antigens may be referred to as a serogroup or sometimes serocomplex.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an subject to whom diagnosis and/or therapy is and/or has been administered.

Susceptible to: A subject who is “susceptible to” a disease, disorder, or condition is at risk for developing the disease, disorder, or condition. In some embodiments, a subject who is susceptible to a disease, disorder, or condition does not display any symptoms of the disease, disorder, or condition. In some embodiments, a subject who is susceptible to a disease, disorder, or condition has not been diagnosed with the disease, disorder, and/or condition. In some embodiments, a subject who is susceptible to a disease, disorder, or condition is a subject who has been exposed to conditions associated with development of the disease, disorder, or condition. In some embodiments, a risk of developing a disease, disorder, and/or condition is a population-based risk (e.g., family members of subjects suffering from the disease, disorder, or condition).

Symptoms are reduced: As used herein, “symptoms are reduced” when one or more symptoms of a particular disease, disorder or condition is reduced in magnitude (e.g., intensity, severity, etc.) and/or frequency, e.g., to a statistically and/or clinically significant or relevant level. For purposes of clarity, a delay in the onset of a particular symptom is considered one form of reducing the frequency of that symptom.

Treatment: As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vaccination: As used herein, the term “vaccination” refers to the administration of a composition intended to generate an immune response, for example to a disease-causing agent. For the purposes of the present invention, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition. In some embodiments, vaccination initiates immunization.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 modifications (e.g., substitutions, additions or deletions) at the N-terminus portion, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 modifications (e.g., substitutions, additions or deletions) at the C-terminus portion, as compared to the reference. In some embodiments, a reference polypeptide or nucleic acid is one found in nature.

### DETAILED DESCRIPTION

The present disclosure relates, generally, to compositions, systems, and methods that include novel complexed proteins and polysaccharides, e.g., vaccines of complexed proteins and polysaccharides. Such complexes can be used, e.g., to induce and/or increase an immunoprotective response in subjects at risk of or suffering from SARS-CoV-2 infection and/or COVID-19.

In one aspect, the present disclosure features novel complexed proteins and polysaccharides, e.g., vaccines of complexed proteins and polysaccharides, and combinations. Such novel complexes and vaccines can be used, e.g., to induce and/or increase an immunoprotective response in subjects, such as those at risk of or suffering from SARS-CoV-2 infection.

SARS-CoV-2 and COVID-19

Coronaviruses are enveloped, positive single stranded RNA viruses. Coronaviruses have been identified in various mammalians hosts such as bats, camels, or mice, among others. Several coronaviruses are pathogenic to human, leading to varying degrees of symptoms severity (Cui et al., Nat Rev Microbiol. 2019 March; 17(3):181-92). Highly pathogenic variants include the severe acute respiratory syndrome coronavirus (SARS-CoV) that emerged in China in 2002, resulting in around 8000 human infections and over 700 deaths (Peiris et al., Nat Med., 2004 December; 10 (12 Suppl):588-97) and the Middle East respiratory syndrome coronavirus (MERS-CoV), first detected in Saudi Arabia in 2012 and responsible for about 2500 human infections and over 850 deaths (Zaki et al., N Engl J Med., 2012 Nov. 8; 367(19): 1814-20; Lee et al., BMC Infect Dis. 2017 Jul. 14; 17(1):498).

As used herein, “SARS-CoV-2”, also referred to herein as “Wuhan coronavirus”, or “Wuhan seafood market pneumonia virus”, or “Wuhan CoV”, or “novel CoV”, or “nCoV”, or “2019 nCoV”, or “Wuhan nCoV”, or “ancestral Wuhan D614G” is a betacoronavirus believed to be of lineage B (sarbecovirus). SARS-CoV-2 was first identified in Wuhan, Hubei province, China, in late 2019 and spread within China and to other parts of the world by early 2020. Symptoms of SARS-CoV-2 include fever, dry cough, and dyspnea. The genomic sequence of SARS-CoV-2 isolate Wuhan-Hu-1 is known (see GenBank MN908947.3, Jan. 23, 2020), and the amino acid translation of the genome is also known (see GenBank QHD43416.1, Jan. 23, 2020) (see also Huang et al., Acta Pharmacologica Sinica 41:1141-1149 (2020)). Those skilled in the art are aware of various strains of SARS-CoV-2. In some embodiments, such variants of SARS-CoV-2 may be identified based on publicly available data (e.g., data provided in the GISAID Initiative database: https://www.gisaid.org, and/or data provided by the World Health Organization WHO (e.g., as provided at https://www.who.int/activities/tracking-SARS-CoV-2-variants).

Immunogenic Complexes

The present disclosure encompasses immunogenic complexes that include one or more polypeptides of SARS-CoV-2 and one or more polysaccharides.

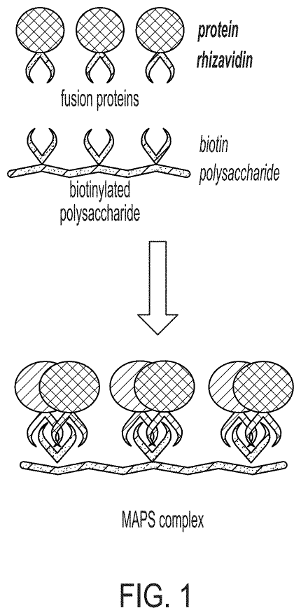

In some embodiments, immunogenic complexes are, or are based on, Multiple Antigen Presenting System (MAPS) complexes. Aspects of the MAPS platform have been previously described in WO2012/155007 and WO2020/056202, the contents of which are herein incorporated by reference in their entirety, and are shown schematically in FIG. 1.

As described herein, immunogenic complexes of the disclosure include one or more antigenic polypeptides non-covalently complexed with one or more antigenic polysaccharides. In some embodiments, one or more antigenic polypeptides are complexed via affinity interaction with one or more antigenic polysaccharides. In some embodiments, immunogenic complexes of the disclosure include one or more antigenic polypeptides non-covalently complexed with one or more antigenic polysaccharides using one or more affinity molecule/complementary affinity molecule pairs. In some embodiments, an immunogenic complex includes (i) a first affinity molecule described herein conjugated to one or more antigenic polysaccharides, and (ii) a fusion protein that is or comprises a complementary affinity molecule described herein and at least one polypeptide associated with SARS-CoV-2. Upon association of the first affinity molecule and the complementary affinity molecule, the one or more antigenic polypeptides are non-covalently complexed to the one or more antigenic polysaccharides.

In some embodiments, one or more antigenic polypeptides are complexed via affinity interaction with one antigenic polysaccharide. In some embodiments, immunogenic complexes of the disclosure include one or more antigenic polypeptides non-covalently complexed with one antigenic polysaccharide using one affinity molecule/complementary affinity molecule pair. In some embodiments, immunogenic complexes of the disclosure include one or more antigenic polypeptides non-covalently complexed with one antigenic polysaccharide using one or more affinity molecule/complementary affinity molecule pairs. In some embodiments, each of the affinity molecule/complementary affinity molecule pairs is the same, e.g., biotin/biotin-binding moiety pairs. In some embodiments, an immunogenic complex includes (i) a first affinity molecule described herein conjugated to one antigenic polysaccharide, and (ii) a fusion protein that is or comprises a complementary affinity molecule described herein and at least one polypeptide associated with SARS-CoV-2. Upon association of the first affinity molecule and the complementary affinity molecule, the one or more antigenic polypeptides are non-covalently complexed to the one antigenic polysaccharide.

In some embodiments, the affinity molecule/complementary affinity molecule pair is selected from one or more of biotin/biotin-binding moiety, antibody/antigen, enzyme/substrate, receptor/ligand, metal/metal-binding protein, carbohydrate/carbohydrate binding protein, lipid/lipid-binding protein, and His tag/His tag-binding molecule.

In some embodiments, the first affinity molecule is biotin (or a derivative or fragment thereof), and the complementary affinity molecule is a moiety, e.g., a biotin-binding protein, or a biotin-binding domain or biotin-binding fragment thereof. In some embodiments, the biotin-binding moiety is rhizavidin, avidin, streptavidin, bradavidin, tamavidin, lentiavidin, zebavidin, NeutrAvidin, CaptAvidin™, or a biotin-binding domain or biotin-binding fragment thereof, or a combination thereof. In some embodiments, the biotin-binding moiety is rhizavidin, or a biotin-binding domain or biotin-binding fragment thereof. In some embodiments, the biotin-binding moiety is or comprises a polypeptide of SEQ ID NO: 1, or a biotin-binding domain or biotin-binding fragment thereof. In some embodiments, the biotin-binding moiety is or comprises a polypeptide of SEQ ID NO: 2, or a biotin-binding domain or biotin-binding fragment thereof. In some embodiments, the biotin-binding moiety is or comprises a polypeptide of SEQ ID NO: 2, or a biotin-binding domain or biotin-binding fragment thereof, or a variant thereof (e.g., comprising one or more mutations).

In some embodiments, the one or more antigenic polysaccharides are, or are derived from Gram-negative bacteria and/or Gram-positive bacteria. In some embodiments, one or more antigenic polysaccharides are, or are derived from one or more glycoproteins. In some embodiments, one or more such glycoproteins are, or are derived from one or more viruses. In some embodiments, one or more bacterial antigenic polysaccharides are, or are derived from S. pneumoniae. In some embodiments, one or more antigenic polysaccharides are, or are derived from one or more pathogens. In some embodiments, one or more antigenic polysaccharides are, or are derived from 1, 2, 3, 4, 5, 6, 7, 8, 9, 10, 11, 12, 13, 14, 15, 16, 17, 18, 19, 20, 21, 22, 23, 24, or 25 serotypes or strains (variants) of a pathogen. In some embodiments, one or more antigenic polysaccharides are, or are derived from more than 25 serotypes or strains (variants) of a pathogen, e.g., 26, 27, 28, 29, 30, 35, 40, 45, or 50 serotypes or strains. In some embodiments, one or more antigenic polysaccharides are, or are derived from more than 60, 70, 80, 90, or 100 serotypes or strains (variants) of a pathogen.

In some embodiments, the one or more antigenic polysaccharides comprise one or more affinity molecules conjugated to the antigenic polysaccharides. In some embodiments, the one or more affinity molecules comprise biotin or biotin derivatives.

In some embodiments, the antigenic polysaccharides comprise a plurality of affinity molecules conjugated to the antigenic polysaccharides. In some embodiments, the affinity molecules comprise biotin or biotin derivatives.

In some embodiments, one or more antigenic polypeptides are covalently linked (e.g., fused) to a complementary affinity molecule described herein. In some embodiments a fusion protein comprises one or more antigenic polypeptides and a complementary affinity molecule disclosed herein. In some embodiments, the complementary affinity molecule is or comprises a biotin-binding moiety. In some embodiments, the complementary affinity molecule is or comprises a dimeric biotin-binding moiety. In some embodiments, the biotin-binding moiety comprises rhizavidin or a biotin-binding portion thereof.

In some embodiments, antigenic polysaccharides and/or antigenic polypeptides that may be included in immunogenic complexes are recombinantly or synthetically produced. In some embodiments, antigenic polysaccharides and/or antigenic polypeptides that may be included in immunogenic complexes are isolated and/or derived from natural sources. In some embodiments antigenic polysaccharides and/or antigenic polypeptides that may be included in immunogenic complexes are isolated from viruses or from bacterial cells. Exemplary polysaccharides and/or polypeptides are described below.

Antigenic Polypeptides

In some embodiments, an immunogenic complex described herein comprises one or more polypeptide antigens. In some embodiments, a polypeptide antigen is a viral polypeptide. In some embodiments, a polypeptide antigen is a polypeptide of, or derived from SARS-CoV-2.

Coronavirus genomes encode non-structural polyprotein and structural proteins, including the Spike (S), Envelope (E), Membrane (M) and Nucleocapsid (N) proteins. In some embodiments, a polypeptide antigen is a Spike (S) protein or antigenic fragment thereof, an Envelope (E) protein or antigenic fragment thereof, a Membrane (M) protein or antigenic fragment thereof; and/or a Nucleocapsid (N) protein or antigenic fragment thereof.

As seen notably with SARS-CoV, neutralizing antibodies and/or T-cell immune responses can be raised against several proteins but mostly target the S glycoprotein, suggesting that S glycoprotein-induced specific immune responses play important parts in the natural response to coronavirus infection (Saif L J, Vet Microbiol. 1993 November; 37(3-4):285-97). The S glycoprotein has key roles in the viral cycle, as it is involved in receptor recognition, virus attachment and entry, and is thus a crucial determinant of host tropism and transmission capacity. Expressed as precursor glycoprotein, S is cleaved in two subunits (S1, which contains the receptor binding domain (RBD), and S2) by proteases.

SEQ ID NO:80 is the amino acid sequence of the Spike (S) glycoprotein of the 2019 novel coronavirus initially named 2019-nCov and renamed SARS-CoV-2 (Severe acute respiratory syndrome coronavirus 2). The S glycoprotein comprises a signal peptide (SP) from position 1 to 18 which is cleaved in the mature S glycoprotein. The S glycoprotein is cleaved into two subunits, S1 which contains the receptor binding domain (RBD) and S2, by proteases. S1 is from positions 19 to 661 of SEQ ID NO:80 and S2 is from positions 662 to 1270 of SEQ ID NO:80. The receptor binding domain (RBD) is from positions 331 to 524 in SEQ ID NO:80. By simple sequence alignment with SEQ ID NO:80, one skilled in the art can easily determine the positions of the RBD in the sequence of a S glycoprotein antigen variant or fragment thereof.

S-RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus S-RBD is believed to interact with ACE2. The amino acid sequence of SARS-CoV-2 Wuhan-Hu-1 S-RBD is provided in SEQ ID NO:81. The amino acid sequence of SARS-CoV-2 Wuhan-Hu-1 RBM is provided in SEQ ID NO:82.

In some embodiments, an antigenic polypeptide has or comprises an amino acid sequence that is at least 90%, at least 91%, at least 92%, at least 93%, at least 94%, at least 95%, at least 96%, at least 97%, at least 98%, at least 99%, or at least 99.5%, or 100% identical to the amino acid sequence of SEQ ID NO:80, 81, or 82, or a portion thereof (e.g., lacking 1, 2, 3, 4, 5, or more amino acids of SEQ ID NO:80, 81, or 82).

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 Spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as20I/501Y.V1 and VOC 202012/01; (del69-70, del144, N501Y, A570D, D614G, P681H, T716I, S982A, and D1118H mutations)) and B.1.351 (also known as 20H/501Y.V2; L18F, D80A, D215G, R246I, K417N, E484K, N501Y, D614G, and A701V mutations), which were discovered in the United Kingdom and South Africa, respectively (Tegally, H., et al., Emergence and rapid spread of a new severe acute respiratory syndrome-related coronavirus 2 (SARS-CoV-2) lineage with multiple Spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p.2020.12.20.20248581). B.1.351 also include two other mutations in the RBD domain of SARS-CoV-2 Spike protein, K417N and E484K (Tegally, H., et al., Emergence and rapid spread of a new severe acute respiratory syndrome-related coronavirus 2 (SARS-CoV-2) lineage with multiple Spike mutations in South Africa. medRxiv, 2020: p. 2020.12.21.20248640). Other SARS-CoV-2 variants include the Lineage B.1.1.28, which was first reported in Brazil; the Variant P.1, lineage B.1.1.28 (also known as20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at reliefweb.int/sites/reliefweb.int/files/resources/2021-jan-20-phe-epi-update-SARS-CoV-2.pdf). Other SARS-CoV-2 variants include a SARS-CoV-2 of clade 19A; SARS-CoV-2 of clade 19B; a SARS-CoV-2 of clade 20A; a SARS-CoV-2 of clade 20B; a SARS-CoV-2 of clade 20C; a SARS-CoV-2 of clade 20D; a SARS-CoV-2 of clade 20E (EU1); a SARS-CoV-2 of clade 20F; a SARS-CoV-2 of clade 20G; and SARS-CoV-2 B.1.1.207; and other SARS-CoV-2 lineages described in Rambaut, A., et al., A dynamic nomenclature proposal for SARS-CoV-2 lineages to assist genomic epidemiology. Nat Microbiol 5,1403-1407 (2020). The foregoing SARS-CoV-2 variants, and the amino acid and nucleotide sequences thereof, are incorporated herein by reference. Those skilled in the art are aware of various SARS-CoV-2 variants and their mutations, for example, relative to a Wuhan strain sequence. In some embodiments, such variants of SARS-CoV-2 may be identified based on publicly available data (e.g., data provided in the GISAID Initiative database: https://www.gisaid.org, and/or data provided by the World Health Organization WHO (e.g., as provided at https://www.who.int/activities/tracking-SARS-CoV-2-variants).

In some embodiments, an antigenic polypeptide is or comprises a SARS-CoV-2 glycoprotein S RBD listed in Table 1 (or an antigenic fragment thereof), or comprises a SARS-CoV-2 glycoprotein S RBD (or an antigenic fragment thereof) that includes one or more mutations listed in Table 1:

TABLE 1
Representative Mutations of SARS-CoV-2 Glycoprotein S RBD [aa 331-524]

| Mutations | | | | | Strain Designation | αα SEQ ID |
|---|---|---|---|---|---|---|
| (reference RBD) | | | | | Wuhan D614G | SEQ ID NO: 3 |
| | | | | N501Y | Alpha, B.1.1.7, UK | SEQ ID NO: 6 |
| K417N | | | E484K | N501Y | Beta, B.1.351, South Africa | SEQ ID NO: 9 |
| | L452R | T478K | | | Delta, B.1.617.2, India | SEQ ID NO: 12 |
| N417N | L452R | T478K | | | Delta plus, AY.1, India | SEQ ID NO: 15 |
| K417T | | | E484K | M501Y | Gamma, P.1, Japan/Brazil | SEQ ID NO: 18 |
| | L452R | | | | Epsilon, B.1.427, B.1.429, CA | SEQ ID NO: 21 |
| | | | E484K | | Eta, B.1.525, UK/Nigeria | SEQ ID NO: 24 |
| | L452R | | E484K | | Iota, B.1.526, US/NY | SEQ ID NO: 27 |
| | L452R | | E484Q | | Kappa, B.1.617.1, India | SEQ ID NO: 30 |
| K417N | L452R | | E484Q | N501Y | Potential VOC #1 | SEQ ID NO: 33 |
| K417N | L452R | | E484K | N501Y | Potential VOC #2 | SEQ ID NO: 36 |
| | L452R | | E484Q | N501Y | Potential VOC #3 | SEQ ID NO: 39 |
| | L452R | | E484K | N501Y | Potential VOC #4 | SEQ ID NO: 42 |

| | | |
|---|---|---|
| Mutations (other) | Strain Designation | αα SEQ ID |

| | | | | |
|---|---|---|---|---|
| | | E484Q | | tbd |
| K417N | | E484Q | | tbd |
| K417N | | E484K | | tbd |
| K417N | L452R | E484K | | tbd |
| | | | N501Y | tbd |
| K417N | | | N501Y | tbd |
| K417N | | E484Q | N501Y | tbd |
| K417N | | E484K | N501Y | tbd |
| K417N | L452R | | N501Y | tbd |

In some embodiments, the disclosure includes nucleic acid sequences encoding any of the amino acids described herein. Due to degeneracy in the genetic code, those of ordinary skill in the art would understand that other DNA sequences (including codon-optimized sequences) could encode these polypeptides, as well as the others disclosed herein.

Fusion Proteins that Include Antigenic Polypeptides

Antigenic polypeptides described herein can be part of a fusion protein. For example, in some embodiments, an immunogenic complex described herein comprises a fusion protein that is or comprises a complementary affinity molecule and one or more antigenic polypeptides described herein. In some embodiments, a fusion protein of the immunogenic complex has carrier properties. In some embodiments, a fusion protein of the immunogenic complex has antigenic properties. In some embodiments, a fusion protein of the immunogenic complex has carrier properties and antigenic properties.

In some embodiments, the fusion protein of the immunogenic complex comprises one or more linkers and/or tags, e.g., a histidine tag. In some embodiments, the linker comprises a polypeptide comprising an amino acid sequence of GGGGSSS (SEQ ID NO:54), GGGGSGGGGSGGGGS (SEQ ID NO:58), or GGGGSGGGGSGGGGSM (SEQ ID NO:59). In some embodiments, the linker comprises a polypeptide comprising an amino acid sequence having at least 80%, 85%, 90%, 95%, 96%, 97%, 98%, or 99% identity to the sequence of GGGGSSS (SEQ ID NO: 54), GGGGSGGGGSGGGGS (SEQ ID NO: 58), or GGGGSGGGGSGGGGSM (SEQ ID NO: 59). In some embodiments, the linker comprises the amino acid sequence AAA. In some embodiments, the fusion protein of the immunogenic complex comprises a first linker comprising a polypeptide comprising the amino acid sequence of GGGGSSS (SEQ ID NO: 54), and a second linker comprising the amino acid sequence AAA. In some embodiments, the fusion protein of the immunogenic complex comprises a first linker comprising a polypeptide comprising the amino acid sequence of GGGGSGGGGSGGGGSM (SEQ ID NO: 59), and a second linker comprising the amino acid sequence AAA. In some embodiments, the fusion protein of the immunogenic complex comprises a first linker comprising a polypeptide comprising the amino acid sequence of GGGGSGGGGSGGGGSM (SEQ ID NO: 59), and a second linker comprising the amino acid sequence GGGGSSS (SEQ ID NO: 54). In some embodiments, such a linker may be synthesized, or derived from amino acid residues from a restriction site (e.g., a Not I restriction site).

Complementary Affinity Molecules

In some embodiments, a complementary affinity molecule comprises a biotin-binding moiety. In some embodiments, a fusion protein of the immunogenic complex comprises a biotin-binding moiety, and one or more polypeptide antigens. In some embodiments, a fusion protein comprises a biotin-binding moiety and two or more polypeptide antigens. As used herein, a “biotin-binding moiety” refers to a biotin-binding protein, a biotin-binding fragment thereof, or a biotin-binding domain thereof. In some embodiments, a biotin-binding moiety is a dimeric biotin-binding protein.

In some embodiments, MAPS complexes disclosed herein utilize the high affinity (dissociation constant [KD]≈10⁻¹⁵M) non-covalent binding between biotin and rhizavidin, a biotin-binding protein that has no significant predicted homology with human proteins. Rhizavidin, a naturally occurring dimeric protein in the avidin protein family, was first discovered in Rhizobium etli, a symbiotic bacterium of the common bean. Rhizavidin has only a 22% amino acid identity with chicken avidin, a protein commonly found in eggs, but with high conservation of amino acid residues involved in biotin binding. No cross-reactivity to rhizavidin is observed in human serum samples obtained from subjects exposed to avidin (Helppolainen et al., Biochem J. 405:397-405 (2007)), suggesting that rhizavidin antibodies may not cross-react with chicken avidin. Biotin conjugates have been used in several clinical applications without any reported adverse events (Buller et al, J Throb Haemost. 12:824-30 (2014); Paty et al, J Thromb Haemost. 8:722-9 (2010); Lazzeri et al, Eur J Nucl Med Mol Imaging. 31:1505-11 (2004)).

In some embodiments, the biotin-binding moiety of the fusion protein comprises rhizavidin or a biotin-binding domain or biotin-binding fragment thereof, as further described in WO 2012/155053 the contents of which are herein incorporated by reference in their entirety. In some embodiments, a biotin-binding moiety is or comprises a polypeptide having at least 80%, 85%, 90%, 95%, 96%, 97%, 98%, or 99% identity to rhizavidin, or a biotin-binding domain or biotin-binding fragment thereof. In some embodiments, the biotin-binding moiety comprises a polypeptide of SEQ ID NO:1 or a biotin-binding domain or biotin-binding fragment thereof (e.g., SEQ ID NO:1 lacking 1, 2, 3, 4, 5, or more amino acids on the N- and/or C-terminus). In some embodiments, the biotin-binding moiety is or comprises a polypeptide having at least 80%, 85%, 90%, 95%, 96%, 97%, 98%, or 99% identity to the sequence of SEQ ID NO:1, or biotin-binding domain or biotin-binding fragment thereof (e.g., lacking 1, 2, 3, 4, 5, or more amino acids on the N- and/or C-terminus). In some embodiments, the biotin-binding moiety is or comprises a polypeptide of SEQ ID NO:2 or a biotin-binding domain or biotin-binding fragment thereof (e.g., SEQ ID NO:2 lacking 1, 2, 3, 4, 5, or more amino acids on the N- and/or C-terminus). In some embodiments, the biotin-binding moiety is or comprises a polypeptide having at least 80%, 85%, 90%, 95%, 96%, 97%, 98%, or 99% identity to the sequence of SEQ ID NO:2, or biotin-binding domain or biotin-binding fragment thereof (e.g., lacking 1, 2, 3, 4, 5, or more amino acids on the N- and/or C-terminus). In some embodiments, the biotin-binding moiety is or comprises a polypeptide of SEQ ID NO: 1 or SEQ ID NO: 2, or a biotin-binding domain or biotin-binding fragment thereof, or a variant thereof (e.g., comprising one or more mutations). In some embodiments, the biotin-binding moiety is or comprises a polypeptide having at least 80%, 85%, 90%, 95%, 96%, 97%, 98%, or 99% identity to the sequence of SEQ ID NO: 1 or SEQ ID NO:2, or a biotin-binding domain or biotin-binding fragment thereof, or a variant thereof (e.g., comprising one or more mutations).

In some embodiments, the fusion protein is or comprises a complementary affinity molecule described herein (e.g., a biotin-binding moiety described herein), and one or more polypeptides of or derived from SARS-CoV-2. In some embodiments, the fusion protein comprises a complementary affinity molecule described herein (e.g., a biotin-binding moiety described herein) and a polypeptide comprising an amino acid sequence having at least 80%, 85%, 90%, 95%, 96%, 97%, 98%, 99%, or 99.5% identity to, or having 100% identity to, the sequence of SEQ ID NO:3, 6, 9, 12, 15, 18, 21, 24, 27, 30, 33, 36, 39 or 42, or an antigenic fragment thereof. In some embodiments, a fusion protein comprises or consists of an amino acid sequence at least 80%, at least 85%, at least 90%, at least 95%, at least 96%, at least 97%, at least 98%, at least 99%, at least 99.5% identical to, or 100% identical to, the sequence of SEQ ID NO:5, 8, 11, 14, 17, 20, 23, 26, 29, 32, 35, 38, 41 or 44, or an antigenic fragment thereof.

Antigenic Polysaccharides

In some embodiments, an antigenic polysaccharide is derived from an organism selected from the group consisting of: bacteria, archaea, viruses, or eukaryotic cells like fungi, insect, plant, or chimeras thereof. In some embodiments, the polysaccharide is derived from a pathogenic bacterium or virus. In some embodiments, the polysaccharide is or is derived from a glycoprotein. In specific embodiments, the polysaccharide is a pneumococcal capsular polysaccharide, a pneumococcal cell-wall polysaccharide, a Salmonella typhi Vi polysaccharide, or a Staphylococcus aureus polysaccharide.

In some embodiments, the polysaccharide is a branched polysaccharide, or alternatively, can be a straight chain polysaccharide.

In some embodiments, an antigenic polysaccharide is a Vi antigen (Salmonella typhi capsular polysaccharide), pneumococcal capsular polysaccharides, pneumococcal cell wall polysaccharide, Hib (Haemophilus influenzae type B) capsular polysaccharide, meningococcal capsular polysaccharides, the polysaccharide of Bacillus anthracis (the causative agent of anthrax), and other bacterial capsular or cell wall polysaccharides, or any combinations thereof.

In some embodiments, the polysaccharide consists of or comprises a sugar moiety. For example, in some embodiments, a polysaccharide is a Vi polysaccharide of Salmonella typhi. The Vi capsular polysaccharide has been developed against bacterial enteric infections, such as typhoid fever. Robbins et al., 150 J. Infect. Dis. 436 (1984); Levine et al., 7 Baillieres Clin. Gastroenterol. 501 (1993). Vi is a polymer of α→1→4-galacturonic acid with an N acetyl at position C-2 and variable O-acetylation at C-3. The virulence of S. typhi correlates with the expression of this molecule. Sharma et al., 101 PNAS 17492 (2004). The Vi polysaccharide vaccine of Salmonella typhi has several advantages: side effects are infrequent and mild, a single dose yields consistent immunogenicity and efficacy. Vi polysaccharide may be reliably standardized by physicochemical methods verified for other polysaccharide vaccines, Vi is stable at room temperature and it may be administered simultaneously with other vaccines without affecting immunogenicity and tolerability. Azze et al., 21 Vaccine 2758 (2003).

The polysaccharide can also be derived from Neisseria meningitidis, e.g., capsular polysaccharides from at least one, two, three or four of the serogroups A, C, W, W135, or Y. In some embodiments, the polysaccharide comprises Type 5, Type 8, or any of the polysaccharides or oligosaccharides of Staphylococcus aureus.

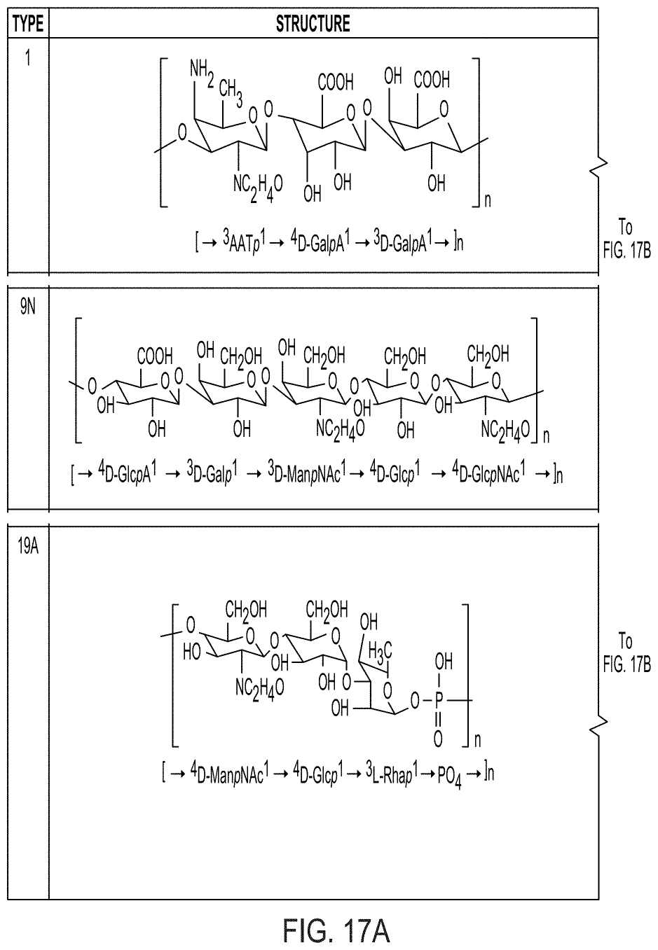

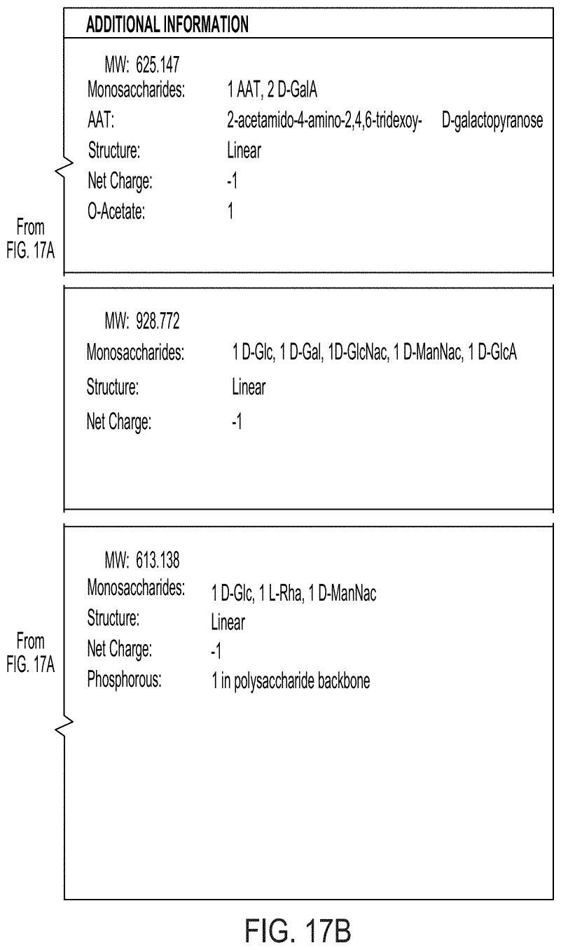

In some embodiments, an immunogenic complex described herein includes one or more S. pneumoniae polysaccharides. In some embodiments, an immunogenic complex described herein includes one S. pneumoniae polysaccharide. Capsular polysaccharides are used to distinguish serotypes of S. pneumoniae. There are at least 97 distinct serotypes of S. pneumoniae polysaccharides, each having a different chemical structure. FIGS. 17A and 17B depict exemplary structures and chemical information for certain S. pneumoniae capsular polysaccharides. All structures are from European Pharmacopoeia 9.0. Serotype designations as used herein are designations according to Danish nomenclature (Kauffmann et al, Intl. Bull. Bact. Nomenclature and Taxonomy 10:31-41 (1960); Geno et al, Clin Microbiol Rev 28(3):871-899 (2015)).

In some embodiments, an immunogenic complex includes one or more S. pneumoniae capsular polysaccharides from, or derived from, one or more S. pneumoniae serotypes selected from 1, 9N, and 19A.

In some embodiments, an immunogenic complex includes one S. pneumoniae capsular polysaccharide from, or derived from, one S. pneumoniae serotype. In some embodiments, an immunogenic complex includes one S. pneumoniae capsular polysaccharide from, or derived from, one S. pneumoniae serotype selected from 1, 9N, and 19A.

In some embodiments, a polysaccharide is harvested and/or purified from a natural source; and in other embodiments, the polysaccharide is synthetic. Methods to produce synthetic polysaccharides are known to persons of ordinary skill and are encompassed in the compositions and methods as disclosed herein.

Methods of Isolating and Purifying Polysaccharides

In some embodiments, the disclosure provides methods of purifying one or more polysaccharides described herein from one or more cellular components of bacteria. In some embodiments, methods comprise purifying capsular polysaccharides from one or more cellular components of bacteria.

In some embodiments, the bacteria are Gram-negative. In some embodiments, the bacteria are Gram-positive. In some embodiments, the bacteria are S. pneumoniae.

In some embodiments, the cellular components include protein. In some embodiments, the cellular proteins include nucleic acid. In some embodiments, the cellular components include lipids. In some embodiments, the cellular components include polysaccharides. In some embodiments, the cellular components are part of a lysate.

In some embodiments, the polysaccharide purification processes incorporate a series of ethanol precipitations, washes of crude polysaccharide preparations with ethanol, diethyl ether, and/or acetone, and drying under vacuum to furnish purified products. In some embodiments, a phenol extraction step is incorporated for polysaccharide purifications. In some embodiments the purification process employs a CTAB (cetyltrimethyl ammonium bromide) precipitation step in addition to using ethanol and phenol precipitation steps.

Methods of Biotinylating Polysaccharides

In some embodiments, the disclosure provides methods of biotinylating one or more polysaccharides described herein. In some embodiments, the method comprises reacting purified polysaccharides with 1-cyano-4-dimethylaminopyridinium tetrafluoroborate (CDAP) for activation of hydroxyl groups in the polysaccharides followed by the addition of amine PEG biotin under conditions that result in covalent linkage of biotin to the polysaccharides. In some embodiments, the desired level of biotinylation is achieved by varying the ratio of CDAP to polysaccharide. In some embodiments, the biotinylated polysaccharides are purified by filtration to remove process residuals such as unreacted biotin, dimethylaminopyridine, acetonitrile, cyanide and unreacted glycine. In some embodiments, the level of polysaccharide biotinylation described herein is optimized to reduce the amount of accessible biotin following MAPS complexation.

Manufacture of Immunogenic Complexes

The present disclosure includes methods for manufacturing immunogenic complexes described herein. In some embodiments, a method of manufacturing immunogenic complexes comprises complexing at least one biotinylated polysaccharide with at least one biotin-binding fusion protein. In some embodiments, the fusion protein comprises an amino acid sequence having at least 80%, at least 85%, at least 90%, at least 95%, at least 96%, at least 97%, at least 98%, at least 99%, or 100% identity to SEQ ID NO:5, 8, 11, 14, 17, 20, 23, 26, 29, 32, 35, 38, 41, or 44.

In some embodiments, the average (e.g., the mean) protein (e.g., antigenic protein) to polysaccharide ratio of a plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average protein to polysaccharide ratio of a plurality of immunogenic complexes is approximately 1:1 (w/w). In some embodiments, the average protein to polysaccharide ratio of a plurality of immunogenic complexes is approximately 2:1 (w/w). In some embodiments, the average protein to polysaccharide ratio of a plurality of immunogenic complexes is approximately 3:1 (w/w). In some embodiments, the average protein to polysaccharide ratio of a plurality of immunogenic complexes is approximately 4:1 (w/w). In some embodiments, the average protein to polysaccharide ratio of a plurality of immunogenic complexes is approximately 5:1 (w/w). In some embodiments, the average protein to polysaccharide ratio of a plurality of immunogenic complexes is approximately 6:1 (w/w). In some embodiments, the average protein to polysaccharide ratio of a plurality of immunogenic complexes is approximately 7:1 (w/w). In some embodiments, the average protein to polysaccharide ratio of a plurality of immunogenic complexes is approximately 8:1 (w/w). In some embodiments, the average protein to polysaccharide ratio of a plurality of immunogenic complexes is approximately 9:1 (w/w). In some embodiments, the average protein to polysaccharide ratio of a plurality of immunogenic complexes is approximately 10:1 (w/w). Immunogenic compositions and vaccines of the invention may comprise mixtures of immunogenic complexes with different average protein to polysaccharide ratios.

In some embodiments, a vaccine or immunogenic composition comprises a plurality of immunogenic complexes comprising (i) a fusion protein comprising or consisting of an amino acid sequence at least 80%, at least 85%, at least 90%, at least 95%, at least 96%, at least 97%, at least 98%, at least 99%, or at least 99.5% identical to any of SEQ ID NO:5, 8, 11, 14, 17, 20, 23, 26, 29, 32, 35, 38, 41 or 44, or an antigenic fragment thereof and (ii) one or more capsular polysaccharides, from or derived from S. pneumoniae serotype 1, 9N, or 19A. In some embodiments, a vaccine or immunogenic composition comprises a plurality of immunogenic complexes comprising (i) a fusion protein comprising or consisting of the amino acid sequence of SEQ ID NO: 5, 8, 11, 14, 17, 20, 23, 26, 29, 32, 35, 38, 41 or 44, or an antigenic fragment thereof and (ii) one or more capsular polysaccharides, from or derived from S. pneumoniae serotype 1, 9N, or 19A. In some embodiments, the average ratio of fusion protein to capsular polysaccharide in the plurality of immunogenic complexes is approximately 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, or 10:1 (weight/weight [w/w]). In some embodiments, the average ratio of fusion protein to capsular polysaccharide in the plurality of immunogenic complexes is approximately 1:1 (w/w). In some embodiments, the average ratio of fusion protein to capsular polysaccharide in the plurality of immunogenic complexes is approximately 2:1 (w/w). In some embodiments, the average ratio of fusion protein to capsular polysaccharide in the plurality of immunogenic complexes is approximately 3:1 (w/w). In some embodiments, the average ratio of fusion protein to capsular polysaccharide in the plurality of immunogenic complexes is approximately 4:1 (w/w). In some embodiments, the average ratio of fusion protein to capsular polysaccharide in the plurality of immunogenic complexes is approximately 5:1 (w/w). In some embodiments, the average ratio of fusion protein to capsular polysaccharide in the plurality of immunogenic complexes is approximately 6:1 (w/w). In some embodiments, the average ratio of fusion protein to capsular polysaccharide in the plurality of immunogenic complexes is approximately 7:1 (w/w). In some embodiments, the average ratio of fusion protein to capsular polysaccharide in the plurality of immunogenic complexes is approximately 8:1 (w/w). In some embodiments, the average ratio of fusion protein to capsular polysaccharide in the plurality of immunogenic complexes is approximately 9:1 (w/w). In some embodiments, the average ratio of fusion protein to capsular polysaccharide in the plurality of immunogenic complexes is approximately 10:1 (w/w). Immunogenic compositions and vaccines of the invention may comprise mixtures of immunogenic complexes with different average protein to polysaccharide ratios.

Immunogenic and Vaccine Compositions

Another aspect of the disclosure provides compositions that include one or more immunogenic complexes described herein. For example, an immunogenic composition, e.g., vaccine composition, can include one or more immunogenic complexes described herein. In some embodiments, such compositions can include a plurality of one type of immunogenic complex described herein. For example, a composition can include a population of one type of immunogenic complex, where all of the immunogenic complexes include the same antigenic polypeptide and the same antigenic polysaccharide. Additionally or alternatively, such compositions can include a plurality of more than one type of immunogenic complex described herein. For example, a composition can include populations of different types of immunogenic complexes. In some embodiments, a composition can include a population of a first type of immunogenic complex and a population of a second type of immunogenic complex, where the first type and the second type of the immunogenic complex have different antigenic polypeptides and/or different antigenic polysaccharides. In some embodiments, a composition can include a population of a first type of immunogenic complex and a population of a second type of immunogenic complex, where the first type and the second type of the immunogenic complex include the same antigenic polypeptide and different antigenic polysaccharides (e.g., polysaccharides of different serotypes). In some embodiments, a composition can include a population of a first type of immunogenic complex and a population of a second type of immunogenic complex, where the first type and the second type of the immunogenic complex include an antigenic polypeptide from different SARS-CoV-2 strains and/or variants and the same or different antigenic polysaccharides (e.g., polysaccharides of different serotypes). In some embodiments, immunogenic complexes described herein are formulated into a pharmaceutical composition. In some embodiments a pharmaceutical composition may be a vaccine. In some embodiments a pharmaceutical composition comprises a pharmaceutically acceptable carrier. In some embodiments a pharmaceutical composition comprises an adjuvant.

Vaccine Compositions

In some embodiments, a vaccine composition is a monovalent vaccine. In some embodiments, a vaccine composition is a polyvalent or multivalent vaccine. In some embodiments, a vaccine composition is a monovariant vaccine, comprising one or more antigens from one strain or variant of a pathogen. In some embodiments, a vaccine composition is a multivariant vaccine, comprising one or more antigens from more than one strain or variant of a pathogen. In some embodiments, the valency of a vaccine composition refers to the number of species of immunogenic complexes present in the vaccine composition. The valency of a vaccine described herein is not limiting with respect to the total antigens present in said pharmaceutical composition, immunogenic complex, or vaccine, or to the number of pathogen strains for which administration of said pharmaceutical composition, immunogenic complex, immunogenic composition, or vaccine composition may induce an immune-protective response.

In some embodiments, a vaccine composition comprises between 1-50 species of immunogenic complexes. In some embodiments, a vaccine composition comprises between 1-40 species of immunogenic complexes. In some embodiments, a vaccine composition comprises between 1-35 species of immunogenic complexes. In some embodiments, a vaccine composition comprises between 1-30 species of immunogenic complexes. In some embodiments, a vaccine composition comprises between 1-30 species of immunogenic complexes. In some embodiments, a vaccine composition comprises between 1-24 species of immunogenic complexes. In some embodiments, a vaccine composition comprises between 1-15 species of immunogenic complexes. In some embodiments, a vaccine composition comprises between 1-9 species of immunogenic complexes. In some embodiments, a vaccine composition comprises between 1-5 species of immunogenic complexes. In some embodiments, a vaccine is a polyvalent vaccine.

In some embodiments, a vaccine composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 species of immunogenic complexes. In some embodiments, a vaccine composition comprises 1 type of immunogenic complex. In some embodiments, a vaccine composition comprises 2 species of immunogenic complexes. In some embodiments, a vaccine composition comprises 4 species of immunogenic complexes. In some embodiments, a vaccine composition comprises 6 species of immunogenic complexes. In some embodiments, a vaccine composition comprises 7 species of immunogenic complexes. In some embodiments, a vaccine composition comprises 8 species of immunogenic complexes. In some embodiments, a vaccine composition comprises 9 species of immunogenic complexes. In some embodiments, a vaccine composition comprises 10 species of immunogenic complexes. In some embodiments, a vaccine composition comprises 11 species of immunogenic complexes. In some embodiments, a vaccine composition comprises 12 species of immunogenic complexes. In some embodiments, a vaccine composition comprises 13 species of immunogenic complexes. In some embodiments, a vaccine composition comprises 14 species of immunogenic complexes. In some embodiments, a vaccine composition comprises 15 species of immunogenic complexes. In some embodiments, a vaccine composition comprises 16 species of immunogenic complexes. In some embodiments, a vaccine composition comprises 17 species of immunogenic complexes. In some embodiments, a vaccine composition comprises 18 species of immunogenic complexes. In some embodiments, a vaccine composition comprises 19 species of immunogenic complexes. In some embodiments, a vaccine composition comprises 20 species of immunogenic complexes. In some embodiments, a vaccine composition comprises 21 species of immunogenic complexes. In some embodiments, a vaccine composition comprises 22 species of immunogenic complexes. In some embodiments, a vaccine composition comprises 23 species of immunogenic complexes. In some embodiments, a vaccine composition comprises 24 species of immunogenic complexes. In some embodiments, a vaccine composition comprises 25 species of immunogenic complexes. In some embodiments, a vaccine composition comprises 26 species of immunogenic complexes. In some embodiments, a vaccine composition comprises 27 species of immunogenic complexes. In some embodiments, a vaccine composition comprises 28 species of immunogenic complexes. In some embodiments, a vaccine composition comprises 29 species of immunogenic complexes. In some embodiments, a vaccine composition comprises 30 species of immunogenic complexes. In some embodiments, a vaccine composition comprises 35 species of immunogenic complexes. In some embodiments, a vaccine composition comprises 40 species of immunogenic complexes. In some embodiments, a vaccine composition comprises 45 species of immunogenic complexes. In some embodiments, a vaccine composition comprises 50 species of immunogenic complexes.

In some embodiments, a vaccine composition comprises two or more species of immunogenic complexes (e.g., in immunogenic compositions) in amounts such that the weight of polysaccharides in the vaccine composition from each immunogenic complex is about the same, e.g., present in a w/w ratio of about 1:1. In some embodiments, the weight of polysaccharides in the vaccine contributed by each immunogenic complex is about 0.20 μg. In some embodiments, the weight of polysaccharides in the vaccine contributed by each immunogenic complex is about 0.25 μg. In some embodiments, the weight of polysaccharides in the vaccine contributed by each immunogenic complex is about 0.5 μg. In some embodiments, the weight of polysaccharides in the vaccine contributed by each immunogenic complex is about 1 μg. In some embodiments, the weight of polysaccharides in the vaccine contributed by each immunogenic complex is about 1.5 μg. In some embodiments, the weight of polysaccharides in the vaccine contributed by each immunogenic complex is about 2 μg. In some embodiments, the weight of polysaccharides in the vaccine contributed by each immunogenic complex is about 2.5 μg. In some embodiments, the weight of polysaccharides in the vaccine contributed by each immunogenic complex is about 3 μg. In some embodiments, the weight of polysaccharides in the vaccine contributed by each immunogenic complex is about 3.5 μg. In some embodiments, the weight of polysaccharides in the vaccine contributed by each immunogenic complex is about 4 μg. In some embodiments, the weight of polysaccharides in the vaccine contributed by each immunogenic complex is about 4.5 μg. In some embodiments, the weight of polysaccharides in the vaccine contributed by each immunogenic complex is about 5 μg. In some embodiments, the weight of polysaccharides in the vaccine contributed by each immunogenic complex is about 5.5 μg. In some embodiments, the weight of polysaccharides in the vaccine contributed by each immunogenic complex is about 6 μg. In some embodiments, the weight of polysaccharides in the vaccine contributed by each immunogenic complex is about 7 μg. In some embodiments, the weight of polysaccharides in the vaccine contributed by each immunogenic complex is about 8 g. In some embodiments, the weight of polysaccharides in the vaccine contributed by each immunogenic complex is about 9 μg. In some embodiments, the weight of polysaccharides in the vaccine contributed by each immunogenic complex is about 10 μg. In some embodiments, the weight of polysaccharides in the vaccine contributed by each immunogenic complex is about 11 μg. In some embodiments, the weight of polysaccharides in the vaccine contributed by each immunogenic complex is about 12 μg. In some embodiments, the weight of polysaccharides in the vaccine contributed by each immunogenic complex is more than 12 μg, e.g., 13 μg, 14 μg, 15 μg, 16 μg, 17 μg, 18 μg, 19 μg, 20 μg, 21 μg, 22 μg, 23 μg, 24 μg, 25 μg, or more.

In some embodiments, a vaccine composition comprises two or more species of immunogenic complexes (e.g., in immunogenic compositions) in amounts such that the weight of polysaccharides in the vaccine composition contributed by each immunogenic complex is different, e.g., present in a w/w ratio that is not about 1:1. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:2. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:3. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:4. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:5. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:6. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:7. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:8. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:9. In some embodiments, a vaccine composition comprises two or more species of immunogenic complexes (e.g., in immunogenic compositions) in amounts such that the weight of polysaccharide in the vaccine composition contributed by a first immunogenic complex and a second immunogenic complex is 1:10. In some embodiments, the vaccine composition comprises a mixture of immunogenic complexes, such that the weight of polysaccharide in a vaccine contributed by an immunogenic complex ranges from about 0.20 μg to about 6 μg. In some embodiments, the vaccine composition comprises a mixture of immunogenic complexes, such that the weight of polysaccharide in a vaccine contributed by an immunogenic complex ranges from about 0.20 μg to about 12 μg. In some embodiments, the vaccine composition comprises a mixture of immunogenic complexes, such that the weight of polysaccharides in the vaccine contributed by each immunogenic complex ranges from about 0.20 μg to about 20 μg. In some embodiments, the vaccine composition comprises a mixture of immunogenic complexes, such that the weight of polysaccharides in the vaccine contributed by each immunogenic complex ranges from about 0.20 μg to about 40 μg.

In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is about the same, e.g., present in a w/w protein:PS ratio of about 1:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 2:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 3:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 4:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 5:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 6:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 7:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 8:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 9:1. In some embodiments, a vaccine composition comprises two or more species of immunogenic complexes (e.g., in immunogenic compositions) in amounts such that the combined weight of polysaccharides and polypeptides in the vaccine composition contributed by each immunogenic complex (e.g., in an immunogenic composition) is present in a w/w protein:PS ratio of about 10:1.

In some embodiments, the combined weight of polysaccharides and polypeptides in the vaccine contributed by each immunogenic complex is about 0.20 μg. In some embodiments, the combined weight of polysaccharides and polypeptides in the vaccine contributed by each immunogenic complex is about 0.40 μg. In some embodiments, the combined weight of polysaccharides and polypeptides in the vaccine contributed by each immunogenic complex is about 1 μg. In some embodiments, the combined weight of polysaccharides and polypeptides in the vaccine contributed by each immunogenic complex is about 2 μg. In some embodiments, the combined weight of polysaccharides and polypeptides in the vaccine contributed by each immunogenic composition is about 3 μg. In some embodiments, the combined weight of polysaccharides and polypeptides in the vaccine contributed by each immunogenic composition is about 4 μg. In some embodiments, the combined weight of polysaccharides and polypeptides in the vaccine contributed by each immunogenic composition is about 5 μg. In some embodiments, the combined weight of polysaccharides and polypeptides in the vaccine contributed by each immunogenic composition is about 6 μg. In some embodiments, the combined weight of polysaccharides and polypeptides in the vaccine contributed by each immunogenic composition is about 7 μg. In some embodiments, the combined weight of polysaccharides and polypeptides in the vaccine contributed by each immunogenic composition is about 8 μg. In some embodiments, the combined weight of polysaccharides and polypeptides in the vaccine contributed by each immunogenic composition is about 9 μg. In some embodiments, the combined weight of polysaccharides and polypeptides in the vaccine contributed by each immunogenic composition is about 10 μg. In some embodiments, the combined weight of polysaccharides and polypeptides in the vaccine contributed by each immunogenic composition is about 11 μg. In some embodiments, the combined weight of polysaccharides and polypeptides in the vaccine contributed by each immunogenic composition is about 12 μg. In some embodiments, the combined weight of polysaccharides and polypeptides in the vaccine contributed by each immunogenic composition is about 14 μg. In some embodiments, the combined weight of polysaccharides and polypeptides in the vaccine contributed by each immunogenic composition is about 16 μg. In some embodiments, the combined weight of polysaccharides and polypeptides in the vaccine contributed by each immunogenic composition is about 18 μg. In some embodiments, the combined weight of polysaccharides and polypeptides in the vaccine contributed by each immunogenic composition is about 20 μg. In some embodiments, the combined weight of polysaccharides and polypeptides in the vaccine contributed by each immunogenic composition is about 21 μg. In some embodiments, the combined weight of polysaccharides and polypeptides in the vaccine contributed by each immunogenic composition is about 22 μg. In some embodiments, the combined weight of polysaccharides and polypeptides in the vaccine contributed by each immunogenic composition is about 23 μg. In some embodiments, the combined weight of polysaccharides and polypeptides in the vaccine contributed by each immunogenic composition is about 24 μg. In some embodiments, the combined weight of polysaccharides and polypeptides in the vaccine contributed by each immunogenic composition is about 25 μg. In some embodiments, the combined weight of polysaccharides and polypeptides in the vaccine contributed by each immunogenic composition is about 30 μg. In some embodiments, the combined weight of polysaccharides and polypeptides in the vaccine contributed by each immunogenic composition is about 40 μg. In some embodiments, the combined weight of polysaccharides and polypeptides in the vaccine contributed by each immunogenic composition is about 50 μg. In some embodiments, the combined weight of polysaccharides and polypeptides in the vaccine contributed by each immunogenic composition is about 60 μg. In some embodiments, the combined weight of polysaccharides and polypeptides in the vaccine contributed by each immunogenic composition is about 70 μg. In some embodiments, the combined weight of polysaccharides and polypeptides in the vaccine contributed by each immunogenic composition is about 80 μg. In some embodiments, the combined weight of polysaccharides and polypeptides in the vaccine contributed by each immunogenic composition is about 90 μg. In some embodiments, the combined weight of polysaccharides and polypeptides in the vaccine contributed by each immunogenic composition is about 100 μg. In some embodiments, the combined weight of polysaccharides and polypeptides in the vaccine contributed by each immunogenic composition is about 110 μg.

In some embodiments, a vaccine composition comprises two or more species of immunogenic complexes (e.g., in immunogenic compositions) in amounts such that the combined weight of polysaccharides and polypeptides in the vaccine composition contributed by each immunogenic complex is different, e.g., present in a w/w protein:PS ratio that is not about 1:1, e.g., a protein:PS ratio that is 2:1, 3:1, 4:1. 5:1. 6:1, 7:1, 8:1, 9:1, or 10:1. In some embodiments, the vaccine composition comprises a mixture of immunogenic complexes, such that the combined weight of polysaccharides and polypeptides in the vaccine contributed by each immunogenic complex ranges from about 0.4 μg to about 110 μg.

Conjugated Immunogenic Complexes; Immunogenic and Vaccine Compositions Comprising Same

In some embodiments, one or more polypeptides (e.g., antigenic polypeptides) of immunogenic complexes are conjugated to one or more polysaccharides. In some embodiments, one or more conjugated polysaccharides comprise a capsular polysaccharide of S. pneumoniae. In some embodiments, one or more polypeptides of conjugated immunogenic complex comprise an antigenic polypeptide of S. pneumoniae. In some embodiments, an antigenic polypeptide of a conjugated immunogenic complex is or comprises a fusion protein. In some such embodiments, a fusion protein of a conjugated immunogenic complex is or comprises a fusion protein comprising a SARS-CoV-2 associated polypeptide or antigenic fragment.

In some embodiments, a conjugated immunogenic complex comprises one or more S. pneumoniae capsular polysaccharides from, or derived from, one or more S. pneumoniae serotypes selected from 1, 9N, and 19A.

Uses of Immunogenic and Vaccine Compositions

In some embodiments, an immunogenic complex described herein that includes one or more antigenic polysaccharides is characterized in that one or more of the opsonization potential, or immune response to one or more antigenic polysaccharides is increased relative to a predetermined level, as measured by ELISA and or by a functional antibody assay. In some embodiments, one or more of the opsonization potential, immune response to the one or more antigenic polysaccharides is increased at least 1-fold, 2-fold, 3-fold, 4-fold, or 5-fold relative to a predetermined level, as measured by ELISA and or by a functional antibody assay. In some embodiments, the predetermined level is a pre-immune level. In some embodiments, the predetermined level is a pre-immune level. In some embodiments, one or more polypeptide antigens are carrier proteins for one or more antigenic polysaccharides.

In some embodiments, an immunogenic complex described herein, upon administration to a subject, induces an immune response against one or more pathogens in the subject at a level greater than a composition comprising an antigenic polysaccharide alone. In some embodiments, an immunogenic complex described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

In some embodiments, an immunogenic complex described herein, upon administration to a subject, induces an immune response against one or more strains (variants) of SARS-CoV-2.

In some embodiments, the immune response is an antibody or B cell response. In some embodiments, the antibody or B cell response is a memory B cell response. In some embodiments, the immune response is a T cell response. In some embodiments, the T cell response is a memory T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or a CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the immune response comprises neutralizing antibodies. In some embodiments, the immune response is a memory response.

In some embodiments, an immunogenic complex described herein, upon administration to a subject, induces antibody production against one or more pathogens in the subject at a level greater than a composition comprising an antigenic polysaccharide alone. In some embodiments, an immunogenic complex described herein, upon administration to a subject, induces antibody production against one or more pathogens in the subject at level greater than a composition comprising a polypeptide antigen alone.

In some embodiments, an immunogenic composition or vaccine described herein, upon administration to a subject, induces an immune response against one or more pathogens in the subject at a level greater than a composition comprising an antigenic polysaccharide alone. In some embodiments, an immunogenic composition or vaccine described herein, upon administration to a subject, induces an immune response against one or more pathogens in the subject at a level greater than a composition comprising a polypeptide antigen alone. In some embodiments, an immunogenic complex described herein, upon administration to a subject, induces a protective immune response.

The SARS-CoV-2 immunogenic compositions and vaccines described herein may be used for prophylactic and/or therapeutic treatment of SARS-CoV-2 infection and/or COVID-19. Accordingly, this application provides a method for immunizing a subject suffering from or susceptible to SARS-CoV-2 infection, comprising administering an immunologically effective amount of any of the immunogenic compositions or vaccine formulations described herein. The subject receiving the vaccination may be a male or a female, and may be an infant, child, adolescent, or adult. In some embodiments, the subject being treated is a human. In other embodiments, the subject is a non-human animal. In some embodiments, an immunogenic complex described herein, upon administration to a subject, induces a protective immune response against SARS-CoV-2 infection and/or COVID-19.

In prophylactic embodiments, a vaccine composition (e.g., ones as described and/or utilized herein) is administered to a subject to induce an immune response that can help protect against the establishment of one or more strains (variants) of SARS-CoV-2, for example by protecting against asymptomatic infection. In some aspects, the method inhibits infection by SARS-CoV-2 in an uninfected subject. In another aspect, the method may reduce transmission, replication, and/or viral load of one or more strains (variants) of SARS-CoV-2 in a subject who is already infected.

In therapeutic embodiments, the vaccine may be administered to a subject suffering from SARS-CoV-2 infection, in an amount sufficient to treat the subject. Treating the subject, in this case, refers to reducing SARS-CoV-2 symptoms and/or viral load and/or sequelae in an infected subject. In some embodiments, treating the subject refers to reducing the duration of symptoms or sequelae, or reducing the intensity of symptoms or sequelae. In some embodiments, the vaccine reduces transmissibility of one or more strains (variants) of SARS-CoV-2 from the vaccinated subject to another subject. In certain embodiments, the reductions described above are at least 25%, 30%, 40%, 50%, 60%, 70%, 80%, or 90%, e.g., relative to a control, e.g., a control subject.

In therapeutic embodiments, the vaccine is administered to a subject post-infection. The vaccine may be administered shortly after infection, e.g. before symptoms or sequelae manifest, or may be administered during or after manifestation of symptoms or sequelae.

In some embodiments, the vaccine compositions of the invention confer protective immunity, allowing a vaccinated subject to exhibit delayed onset of symptoms or sequelae, or reduced severity of symptoms or sequelae, as the result of his or her exposure to the vaccine. In certain embodiments, the reduction in severity of symptoms or sequelae is at least 25%, 40%, 50%, 60%, 70%, 80%, or 90%, e.g., relative to a control. In particular embodiments, vaccinated subjects may display no symptoms or sequelae upon infection with SARS-CoV-2 (asymptomatic infection), or do not become infected by SARS-CoV-2. Protective immunity is typically achieved by one or more of the following mechanisms: mucosal, humoral, or cellular immunity. Mucosal immunity is primarily the result of secretory IgA (sIGA) antibodies on mucosal surfaces of the respiratory, gastrointestinal, and genitourinary tracts. The sIGA antibodies are generated after a series of events mediated by antigen-processing cells, B and T lymphocytes, that result in sIGA production by B lymphocytes on mucosa-lined tissues of the body. Humoral immunity is typically the result of IgG antibodies and IgM antibodies in serum. Cellular immunity can be achieved through cytotoxic T lymphocytes or through delayed-type hypersensitivity that involves macrophages and T lymphocytes, as well as other mechanisms involving T cells without a requirement for antibodies. In particular, cellular immunity may be mediated by T_{H}1 or T_{H}17 cells.

In some embodiments, an immunogenic composition or vaccine described herein, upon administration to a subject, induces an immune response against one or more strains (variants) of SARS-CoV-2.

In some embodiments, the immune response is an antibody or B cell response. In some embodiments, the antibody or B cell response is a memory B cell response. In some embodiments, the immune response is a T cell response. In some embodiments, the T cell response is a memory T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the immune response comprises neutralizing antibodies. In some embodiments, the immune response is a memory response.

In some embodiments, an immunogenic composition or vaccine described herein, upon administration to a subject, induces an antibody or B cell response against one or more pathogens in the subject at a level greater than a composition comprising an antigenic polysaccharide alone. In some embodiments, an immunogenic composition or vaccine described herein, upon administration to a subject, induces an antibody or B cell response against one or more pathogens in the subject at level greater than a composition comprising a polypeptide antigen alone. In some embodiments, the immune response is a protective immune response. In some embodiments, the immune response comprises neutralizing antibodies.

In some embodiments, an immunogenic composition or vaccine described herein, upon administration to a subject, induces a T cell response against one or more pathogens in the subject at a level greater than a composition comprising an antigenic polysaccharide alone. In some embodiments, an immunogenic composition or vaccine described herein, upon administration to a subject, induces a T cell response against one or more pathogens in the subject at level greater than a composition comprising a polypeptide antigen alone. In some embodiments, the immune response is a protective immune response. In some embodiments, the immune response comprises neutralizing antibodies.

In some embodiments, upon administration to a subject, an immunogenic composition or vaccine described herein treats or prevents infection by one or more strains (variants) of SARS-CoV-2. In some embodiments, upon administration to a subject, an immunogenic composition or vaccine described herein treats or prevents COVID-19 due to infection by one or more strains (variants) of SARS-CoV-2. In some embodiments, upon administration to a subject, an immunogenic composition or vaccine described herein treats or prevents pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2.

In some embodiments, upon administration to a subject, an immunogenic composition or vaccine described herein inhibits or reduces the rate of occurrence of infection by one or more strains (variants) of SARS-CoV-2. In some embodiments, upon administration to a subject, an immunogenic composition or vaccine described herein inhibits or reduces the rate of occurrence of COVID-19 due to infection by one or more strains (variants) of SARS-CoV-2. In some embodiments, upon administration to a subject, an immunogenic composition or vaccine described herein inhibits or reduces the rate of occurrence of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2.

In some embodiments, upon administration to a subject, an immunogenic composition or vaccine described herein reduces the severity of infection by one or more strains (variants) of SARS-CoV-2. In some embodiments, upon administration to a subject, an immunogenic composition or vaccine described herein reduces the severity of COVID-19 due to infection by one or more strains (variants) of SARS-CoV-2. In some embodiments, upon administration to a subject, an immunogenic composition or vaccine described herein reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2.

In some embodiments, upon administration to a subject, an immunogenic composition or vaccine described herein inhibits transmission of one or more strains (variants) of SARS-CoV-2 from the subject to another subject. In some embodiments, upon administration to a subject, an immunogenic composition or vaccine described herein inhibits asymptomatic infection by one or more strains (variants) of SARS-CoV-2 in the subject. In some embodiments, upon administration to a subject, an immunogenic composition or vaccine described herein inhibits replication and/or reduces viral load of one or more strains (variants) of SARS-CoV-2 in the subject.

In some embodiments, an immunogenic composition or vaccine described herein, upon administration to a subject, induces an immune response against one or more pathogens in the subject at a level greater than a control composition. In some embodiments, an immunogenic composition or vaccine described herein, upon administration to a subject, induces a protective immune response against one or more pathogens in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments the subject is a human. In some embodiments the human is between about 2 weeks of age and about 6 weeks of age. In some embodiments the human is between about 6 weeks of age and about 6 years of age. In some embodiments the human is between about 6 years of age and about 18 years of age. In some embodiments the human is between about 18 years of age and about 50 years of age. In some embodiments the human is about 50 years of age and about 75 years of age. In some embodiments, the human is about 75 years of age or older.

In some embodiments, an immunogenic composition or vaccine described herein, upon administration to a subject, induces an immune response against one or more strains (variants) of SARS-CoV-2 at a level greater than a control composition. In some embodiments, the immune response is an antibody or B cell response. In some embodiments, the immune response is a T cell response. In some embodiments, the immune response is an innate immune response. In some embodiments, the immune response is a CD4+ T cell response, including T_{H}1, T_{H}2, or T_{H}17 response, or a CD8+ T cell response, or a CD4+ and CD8+ T cell response, or CD4−/CD8− T cell response. In some embodiments, the immune response is an antibody or B cell response, and a T cell response. In some embodiments, the immune response is an antibody or B cell response, a T cell response, and an innate immune response. In some embodiments, the immune response is a protective immune response. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine described herein, upon administration to a subject, induces an antibody or B cell response against one or more pathogens in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine described herein, upon administration to a subject, induces an antibody or B cell response against one or more pathogens in the subject at level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine described herein, upon administration to a subject, induces a T cell response against one or more pathogens in the subject at a level greater than a control composition. In some embodiments, an immunogenic composition or vaccine described herein, upon administration to a subject, induces a T cell response against one or more pathogens in the subject at level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition or vaccine described herein treats or prevents infection by one or more strains (variants) of SARS-CoV-2 in the subject at a level greater than a control composition. In some embodiments, upon administration to a subject, an immunogenic composition or vaccine described herein treats or prevents COVID-19 due to infection by one or more strains (variants) of SARS-CoV-2 in the subject at a level greater than a control composition. In some embodiments, upon administration to a subject, an immunogenic composition or vaccine described herein treats or prevents pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2 in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition or vaccine described herein inhibits or reduces the rate of occurrence of infection by one or more strains (variants) of SARS-CoV-2 in the subject at a level greater than a control composition. In some embodiments, upon administration to a subject, an immunogenic composition or vaccine described herein inhibits or reduces the rate of occurrence of COVID-19 due to infection by one or more strains (variants) of SARS-CoV-2 in the subject at a level greater than a control composition. In some embodiments, upon administration to a subject, an immunogenic composition or vaccine described herein inhibits or reduces the rate of occurrence of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2 in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition or vaccine described herein reduces the severity of infection by one or more strains (variants) of SARS-CoV-2 in the subject at a level greater than a control composition. In some embodiments, upon administration to a subject, an immunogenic composition or vaccine described herein reduces the severity of COVID-19 due to infection by one or more strains (variants) of SARS-CoV-2 in the subject at a level greater than a control composition. In some embodiments, upon administration to a subject, an immunogenic composition or vaccine described herein reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms due to infection by one or more strains (variants) of SARS-CoV-2 in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, upon administration to a subject, an immunogenic composition or vaccine described herein inhibits transmission of one or more strains (variants) of SARS-CoV-2 from the subject to another subject at a level greater than a control composition. In some embodiments, upon administration to a subject, an immunogenic composition or vaccine described herein inhibits asymptomatic infection by one or more strains (variants) of SARS-CoV-2 in the subject at a level greater than a control composition. In some embodiments, upon administration to a subject, an immunogenic composition or vaccine described herein inhibits replication and/or reduces viral load of one or more strains (variants) of SARS-CoV-2 in the subject at a level greater than a control composition. In some embodiments, the level greater is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, or about 95% of the control composition.

In some embodiments, an immunogenic composition or vaccine described herein is administered to a subject between about 6 weeks and about 5 years (e.g., prior to the 6^{th} birthday) for active immunization for the prevention of COVID-19 caused by one or more strains (variants) of SARS-CoV-2.

In some embodiments an immunogenic composition or vaccine described herein is administered to a subject between about 6 years and about 17 years (e.g., prior to the 18^{th} birthday) for active immunization for the prevention of COVID-19 caused by one or more strains (variants) of SARS-CoV-2.

In some embodiments an immunogenic composition or vaccine described herein is administered to a subject 18 years or older for active immunization for the prevention of COVID-19 caused by one or more strains (variants) of SARS-CoV-2.

Antibody Compositions

Some embodiments provide for an antibody composition comprising antibodies raised in a mammal immunized with an immunogenic complex of the invention. In some embodiments, an antibody comprises at least one antibody selected from the group consisting of mAbs and anti-idiotype antibodies. In some embodiments, an antibody composition comprises neutralizing antibodies. In some embodiments, an antibody composition comprises an isolated gamma globulin fraction. In some embodiments, an antibody composition comprises polyclonal antibodies. In some embodiments, the antibody composition is administered to a subject. In some embodiments, the antibody composition administered to a subject confers passive immunization.

Vaccine Formulations

Optimal amounts of components for a particular vaccine can be ascertained by standard studies involving observation of appropriate immune responses in subjects. Following an initial vaccination, subjects can receive one or several booster immunizations adequately spaced in time.

The immunogenic complexes described herein, and/or preparations thereof may be formulated in a unit dosage form for ease of administration and uniformity of dosage. The specific therapeutically effective dose level for any particular subject or organism may depend upon a variety of factors including the severity or degree of risk of infection; the activity of the specific vaccine or vaccine composition employed; other characteristics of the specific vaccine or vaccine composition employed; the age, body weight, general health, sex of the subject, diet of the subject, pharmacokinetic condition of the subject, the time of administration (e.g., with regard to other activities of the subject such as eating, sleeping, receiving other medicines including other vaccine doses, etc.), route of administration, rate of excretion of the specific vaccine or vaccine composition employed; vaccines used in combination or coincidental with the vaccine composition employed; and like factors well known in the medical arts.

Immunogenic complexes for use in accordance with the present disclosure may be formulated into compositions (e.g., pharmaceutical compositions) according to known techniques. Vaccine preparation is generally described in Vaccine Design (Powell and Newman, 1995). For example, an immunologically amount of a vaccine product can be formulated together with one or more organic or inorganic, liquid or solid, pharmaceutically suitable carrier materials.

In general, pharmaceutically acceptable carrier(s) include solvents, dispersion media, and the like, which are compatible with pharmaceutical administration. For example, materials that can serve as pharmaceutically acceptable carriers include, but are not limited to sugars such as lactose, glucose, dextrose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; polyols such as glycerol, propylene glycol, and liquid polyethylene glycol; esters such as ethyl oleate and ethyl laurate; agar; buffering agents such as magnesium hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as preservatives, and antioxidants can also be present in the composition, according to the judgment of the formulator (Martin, 1975).

Vaccines may be formulated by combining one or more of the immunogenic complexes disclosed herein with carriers and/or other optional components by any available means including, for example, conventional mixing, granulating, dissolving, lyophilizing, or similar processes.

Vaccine compositions useful in the provided methods may be lyophilized up until they are about to be used, at which point they are extemporaneously reconstituted with diluent. In some embodiments, vaccine components or compositions are lyophilized in the presence of one or more other components (e.g., adjuvants), and are extemporaneously reconstituted with saline solution. Alternatively, individual components, or sets of components may be separately lyophilized and/or stored (e.g., in a vaccination kit), the components being reconstituted and either mixed prior to use or administered separately to the subject.

Lyophilization can produce a more stable composition (for instance by preventing or reducing breakdown of polysaccharide antigens). Lyophilizing of vaccines or vaccine components is well known in the art. Typically, a liquid vaccine or vaccine component is freeze dried, often in the presence of an anti-caking agent (such as, for example, sugars such as sucrose or lactose). In some embodiments, the anti-caking agent is present, for example, at an initial concentration of 10-200 mg/mL. Lyophilization typically occurs over a series of steps, for instance a cycle starting at −69° C., gradually adjusting to −24° C. over 3 hours, then retaining this temperature for 18 hours, then gradually adjusting to −16° C. over 1 hour, then retaining this temperature for 6 hours, then gradually adjusting to +34° C. over 3 hours, and finally retaining this temperature over 9 hours.

In some embodiments, a vaccine is a liquid. In some embodiments the liquid is a reconstituted lyophylate. In some embodiments a vaccine has a pH of about 5, about 6, about 7, or about 8. In some embodiments a vaccine has a pH between about 5 and about 7.5. In some embodiments a vaccine has a pH between 5 and 7.5. In some embodiments a vaccine has a pH between about 5.3 and about 6.3. In some embodiments a vaccine has a pH between 5.3 and 6.3. In some embodiments a vaccine has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

Vaccines or vaccine components for use in accordance with the present invention may be incorporated into liposomes, cochleates, biodegradable polymers such as poly-lactide, poly-glycolide and poly-lactide-co-glycolides, or immune-stimulating complexes (ISCOMs).

In certain situations, it may be desirable to prolong the effect of a vaccine or for use in accordance with the present invention, for example by slowing the absorption of one or more vaccine components. Such delay of absorption may be accomplished, for example, by the use of a liquid suspension of crystalline or amorphous material with poor water solubility. The rate of absorption of the product then depends upon its rate of dissolution, which in turn, may depend upon size and form. Alternatively, or additionally, delayed absorption may be accomplished by dissolving or suspending one or more vaccine components in an oil vehicle. Injectable depot forms can also be employed to delay absorption. Such depot forms can be prepared by forming microcapsule matrices of one or more vaccine components a biodegradable polymers network. Depending upon the ratio of polymer to vaccine component, and the nature of the particular polymer(s) employed, the rate of release can be controlled.

Examples of biodegradable polymers that can be employed in accordance with the present invention include, for example, poly(orthoesters) and poly(anhydrides). One particular exemplary polymer is polylactide-polyglycolide.

Depot injectable formulations may also be prepared by entrapping the product in liposomes or microemulsions, which are compatible with body tissues.

Polymeric delivery systems can also be employed in non-depot formulations including, for example, oral formulations. For example, biodegradable, biocompatible polymers such as ethylene vinyl acetate, polyanhydrides, polyglycolic acid, collagen, polyorthoesters, and polylactic acid, etc., can be used in oral formulations. Polysaccharide antigens or conjugates may be formulated with such polymers, for example to prepare particles, microparticles, extrudates, solid dispersions, admixtures, or other combinations in order to facilitate preparation of useful formulations (e.g., oral).

Vaccines for use in accordance with the present invention include immunogenic compositions, and may additionally include one or more additional active agents (i.e., agents that exert a biological effect—not inert ingredients). For example, it is common in vaccine preparation to include one or more adjuvants. It will be appreciated that such additional agents may be formulated together with one or more other vaccine components, or may be maintained separately and combined at or near the time of administration. In some embodiments, such additional components may be administered separately from some or all of the other vaccine components, within an appropriate time window for the relevant effect to be achieved.

Adjuvants

The vaccine formulations and immunogenic compositions described herein may include an adjuvant. Adjuvants, generally, are agents that enhance the immune response to an antigen. Adjuvants can be broadly separated into two classes, based on their principal mechanisms of action: vaccine delivery systems and immunostimulatory adjuvants (see, e.g., Singh et al, 2003). In most vaccine formulations, the adjuvant provides a signal to the immune system so that it generates a response to the antigen, and the antigen is required for driving the specificity of the response to the pathogen. Vaccine delivery systems are often particulate formulations, e.g., emulsions, microparticles, immune-stimulating complexes (ISCOMs), nanoparticles, which may be, for example, particles and/or matrices, and liposomes. In contrast, immunostimulatory adjuvants are sometimes from or derived from pathogens and can represent pathogen associated molecular patterns (PAMP), e.g., lipopolysaccharides (LPS), monophosphoryl lipid A (MPL), or CpG-containing DNA, which activate cells of the innate immune system.

Alternatively, adjuvants may be classified as organic and inorganic. Inorganic adjuvants include alum salts such as aluminum phosphate, amorphous aluminum hydroxyphosphate sulfate, and aluminum hydroxide, which are commonly used in human vaccines. Organic adjuvants comprise organic molecules including macromolecules. Non-limiting examples of organic adjuvants include cholera toxin/toxoids, other enterotoxins/toxoids or labile toxins/toxoids of Gram-negative bacteria, interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), and tumor necrosis factor (TNF).

Adjuvants may also be classified by the response they induce. In some embodiments, the adjuvant induces the generation, proliferation, or activation of T_{H}1 cells or T_{H}2 cells. In other embodiments, the adjuvant induces the generation, proliferation, or activation of B cells. In yet other embodiments, the adjuvant induces the activation of antigen-presenting cells. These categories are not mutually exclusive; in some cases, an adjuvant activates more than one type of cell.

In certain embodiments, the adjuvant induces the generation, proliferation, or activation of T_{H}17 cells. The adjuvant may promote the CD4+ or CD8+ T cells to secrete IL-17. In some embodiments, an adjuvant that induces the generation, proliferation, or activation of T_{H}17 cells is one that produces at least a 2-fold, and in some cases a 10-fold, experimental sample to control ratio in the following assay. In the assay, an experimenter compares the IL-17 levels secreted by two populations of cells: (1) cells from animals immunized with the adjuvant and a polypeptide known to induce T_{H}17 generation, proliferation, or activation, and (2) cells from animals treated with the adjuvant and an irrelevant (control) polypeptide. An adjuvant that induces the generation, proliferation, or activation of T_{H}17 cells may cause the cells of population (1) to produce more than 2-fold, or more than 10-fold more IL-17 than the cells of population (2). IL-17 may be measured, for example, by ELISA or ELISPOT. Certain toxins, such as cholera toxin and labile toxin (produced by enterotoxigenic E. coli, or ETEC), activate a T_{H}17 response. Thus, in some embodiments, the adjuvant is a toxin or toxoid. Mutant derivates of labile toxin (toxoids) that are active as adjuvants but significantly less toxic can be used as well. Exemplary detoxified mutant derivatives of labile toxin include mutants lacking ADP-ribosyltransferase activity. Particular detoxified mutant derivatives of labile toxin include LTK7 (Douce et al, 1995) and LTK63 (Williams et al, 2004), LT-G192 (Douce et al, 1999), and LTR72 (Giuliani et al, 1998).

In some embodiments, the adjuvant comprises a VLP (virus-like particle). One such adjuvant platform, Alphavirus replicons, induces the activation of T_{H}17 cells using alphavirus and is produced by Alphavax. In certain embodiments of the Alphavirus replicon system, alphavirus may be engineered to express an antigen of interest, a cytokine of interest (for example, IL-17 or a cytokine that stimulates IL-17 production), or both, and may be produced in a helper cell line. More detailed information may be found in U.S. Pat. Nos. 5,643,576 and 6,783,939. In some embodiments, a vaccine formulation is administered to a subject in combination with a nucleic acid encoding a cytokine.

Certain classes of adjuvants activate toll-like receptors (TLRs) in order to activate a T_{H}17 response. TLRs are well known proteins that may be found on leukocyte membranes, and recognize foreign antigens (including microbial antigens). Administering a known TLR ligand together with an antigen of interest (for instance, as a fusion protein) can promote the development of an immune response specific to the antigen of interest. One exemplary adjuvant that activates TLRs comprises Monophosphoryl Lipid A (MPL). Traditionally, MPL has been produced as a detoxified lipopolysaccharide (LPS) endotoxin obtained from Gram-negative bacteria, such as S. minnesota. In particular, sequential acid and base hydrolysis of LPS produces an immunoactive lipid A fraction (which is MPL), and lacks the saccharide groups and all but one of the phosphates present in LPS. A number of synthetic TLR agonists (in particular, TLR-4 agonists) are disclosed in Evans et al, 2003. Like MPL adjuvants, these synthetic compounds activate the innate immune system via TLR. Another type of TLR agonist is a synthetic phospholipid dimer, for example E6020 (Ishizaka et al, 2007). Various TLR agonists (including TLR-4 agonists) have been produced and/or sold by, for example, the Infectious Disease Research Institute (IRDI), Corixa, Esai, Avanti Polar Lipids, Inc., and Sigma Aldrich. Another exemplary adjuvant that activates TLRs comprises a mixture of MPL, Trehalose Dicoynomycolate (TDM), and dioctadecyldimethylammonium bromide (DDA). Another TLR-activating adjuvant is R848 (resiquimod).

In some embodiments, the adjuvant is or comprises a saponin. Typically, the saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C.

In certain embodiments, combinations of adjuvants are used. Three exemplary combinations of adjuvants are MPL and alum, E6020 and alum, and MPL and an ISCOM.

Adjuvants may be covalently or non-covalently bound to antigens. In some embodiments, the adjuvant may comprise a protein which induces inflammatory responses through activation of antigen-presenting cells (APCs). In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (e.g., see Wu et al, 2005).

In some embodiments, immunogenic complexes described herein are formulated and/or administered in combination with an adjuvant. In some embodiments, the adjuvant is selected from the group consisting of aluminum phosphate, aluminum hydroxide, and phosphate aluminum hydroxide. In some embodiments, the adjuvant comprises aluminum phosphate. In some embodiments, the adjuvant is aluminum phosphate.

Typically, the same adjuvant or mixture of adjuvants is present in each dose of a vaccine. Optionally, however, an adjuvant may be administered with the first dose of vaccine and not with subsequent doses (i.e., booster shots). Alternatively, a strong adjuvant may be administered with the first dose of vaccine and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human subjects, non-human animals, or both.

Vaccines for use in accordance with the present invention may include, or be administered concurrently with, other antimicrobial, antiviral, or anti-inflammatory therapies. For example, such vaccines may include or be administered with one or more agents that kills or retards growth of a pathogen. Such agents include, for example, remdesivir, lopinavir and/or ritonavir (e.g., Kaletra), oseltamivir (Tamiflu), favipiravir, umifenovir, galidesivir, dexamethasone, colchicine, convalescent plasma, monoclonal antibodies (e.g., one or more of bamlanivimab, LY-CoV016, etesevimab, casirivimab, indevimab, sarilumab, tocilizumab), IL-6 inhibitors, kinase inhibitors, interferons, penicillin, vancomycin, erythromycin, azithromycin, and clarithromycin, cefotaxime, ceftriaxone, levoflaxin, gatifloxacin.

Alternatively or additionally, vaccines for use in accordance with the present invention may include, or be administered with, one or more other vaccines or therapies. For example, one or more non-SARS-CoV-2 antigens may be included in or administered with the vaccines.

Additional Components and Excipients

In addition to the antigens and the adjuvants described above, a vaccine formulation or immunogenic composition may include one or more additional components.

In certain embodiments, the vaccine formulation or immunogenic composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, the vaccine formulation or immunogenic composition includes one or more buffers such as a mixture of sodium bicarbonate and ascorbic acid. In some embodiments, the vaccine formulation may be administered in saline, such as phosphate buffered saline (PBS), or distilled water.

In certain embodiments, the vaccine formulation or immunogenic composition includes one or more surfactants, for example, but not limited to, polysorbate 80 (TWEEN 80), polysorbate 20 (TWEEN 20), Polyethylene glycol p-(1,1,3,3-tetramethylbutyl)-phenyl ether (TRITON X-100), and 4-(1,1,3,3-Tetramethylbutyl)phenol polymer with formaldehyde and oxirane (TYLOXAPOL). A surfactant can be ionic or non-ionic.

In certain embodiments, the vaccine formulation or immunogenic composition includes one or more salts such as sodium chloride, ammonium chloride, calcium chloride, or potassium chloride.

In certain embodiments, a preservative is included in the vaccine or immunogenic composition. In other embodiments, no preservative is used. A preservative is most often used in multi-dose vaccine vials, and is less often needed in single-dose vaccine vials. In certain embodiments, the preservative is 2-phenoxyethanol, methyl and propyl parabens, benzyl alcohol, and/or sorbic acid.

Methods of Administration

In some embodiments, immunogenic complexes are administered to a subject at risk of developing disease caused by SARS-CoV-2, e.g. an infant, a toddler, a juvenile, an adult, or an older adult. In some embodiments the subject is a human. In some embodiments the human is between about 2 weeks of age and about 6 weeks of age. In some embodiments the human is between about 6 weeks of age and about 6 years of age. In some embodiments the human is between about 6 years of age and about 18 years of age. In some embodiments the human is between about 18 years of age and about 50 years of age. In some embodiments the human is about 50 years of age or older. In some embodiments, immunogenic complexes are administered to a subject at elevated risk of developing disease caused by SARS-CoV-2, e.g., immunocompromised subjects, subjects having diseases associated with treatment with immunosuppressive drugs or radiation therapy (including malignant neoplasm, leukemia, lymphomas, Hodgkin's disease, or solid organ transplantation), congenital or acquired immunodeficiency, HIV infection, chronic heart disease, chronic lung disease, diabetes mellitus, chronic liver disease, cigarette smoking, asthma, generalized malignancy, multiple myeloma, or solid organ transplantation. It will be appreciated that a subject can be considered at risk for developing a disease without having been diagnosed with any symptoms of the disease. For example, if the subject is known to have been, or to be intended to be, in situations with relatively high risk of infection, that subject will be considered at risk for developing the disease.

Any effective route of administration may be utilized such as, for example, oral, nasal, enteral, parenteral, intramuscular or intravenous, subcutaneous, transdermal, intradermal, rectal, vaginal, topical, ocular, pulmonary, or by contact application. In some embodiments, vaccine compositions may be injected (e.g., via intramuscular, intraperitoneal, intradermal and/or subcutaneous routes); or delivered via the mucosa (e.g., to the oral/alimentary, respiratory, and/or genitourinary tracts). Intranasal administration of vaccines may be particularly useful in some contexts, for example for prevention or treatment of COVID-19-related pneumonia. In some embodiments of the invention, it may be desirable to administer different doses of a vaccine by different routes; in some embodiments, it may be desirable to administer different components of one dose via different routes. In some embodiments, an immunogenic composition or vaccine disclosed herein is administered intramuscularly. In some embodiments, an immunogenic composition or vaccine disclosed herein is administered subcutaneously.

In some embodiments of the present invention, pharmaceutical compositions (e.g., vaccines) are administered intradermally. Conventional technique of intradermal injection, the “Mantoux procedure”, comprises steps of cleaning the skin, and then stretching with one hand, and with the bevel of a narrow gauge needle (26-31 gauge) facing upwards the needle is inserted at an angle of between 10-15°. Once the bevel of the needle is inserted, the barrel of the needle is lowered and further advanced while providing a slight pressure to elevate it under the skin. The liquid is then injected very slowly thereby forming a bleb or bump on the skin surface, followed by slow withdrawal of the needle.

Devices that are specifically designed to administer liquid agents into or across the skin have been described, for example the devices described in WO 99/34850 and EP 1092444, also the jet injection devices described for example in WO 01/13977; U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 97/37705 and WO 97/13537. Other methods of intradermal administration of the vaccine preparations may include conventional syringes and needles, or devices designed for ballistic delivery of solid vaccines (WO 99/27961), or transdermal patches (WO 97/48440; WO 98/28037); or applied to the surface of the skin (transdermal or transcutaneous delivery WO 98/20734; WO 98/28037).

As described above, pharmaceutical compositions (e.g., vaccines) may be administered as a single dose or as multiple doses. It will be appreciated that an administration is a single “dose” so long as all relevant components are administered to a subject within a window of time; it is not necessary that every component be present in a single composition. For example, administration of two different immunogenic compositions, within a period of less than 24 hours, is considered a single dose. To give but one example, immunogenic compositions having different antigenic components may be administered in separate compositions, but as part of a single dose. As noted above, such separate compositions may be administered via different routes or via the same route. Alternatively or additionally, in embodiments wherein a vaccine comprises a combination of immunogenic compositions and additional types of active agents, immunogenic compositions may be administered via one route, and a second active agent may be administered by the same route or by a different route.

Pharmaceutical compositions (e.g., vaccines) are administered in such amounts and for such time as is necessary to achieve a desired result. In certain embodiments of the present invention, a vaccine composition comprises an immunologically effective amount of at least immunogenic composition. The exact amount required to achieve an immunologically effective amount may vary, depending on the immunogenic composition, and from subject to subject, depending on the species, age, and general condition of the subject, the stage of the disease, the particular pharmaceutical mixture, its mode of administration, and the like.

The amount of polypeptide antigen(s), polysaccharide antigen(s) or conjugate(s) in each pharmaceutical composition (e.g., vaccine) dose is selected to allow the vaccine, when administered as described herein, to induce an appropriate immune-protective response without significant, adverse side effects.

In some embodiments, a pharmaceutical composition comprising an immunogenic complex disclosed herein induces a T_{H}1 and/or T_{H}17 cell response upon administration to a subject. In some embodiments, a pharmaceutical composition comprising an immunogenic complex disclosed herein induces neutralizing antibodies against one or more strains (variants) of SARS-CoV-2 upon administration to a subject. In some embodiments, a pharmaceutical composition comprising an immunogenic complex disclosed herein reduces rate of transmission, replication, and/or viral load of one or more strains (variants) of SARS-CoV-2 upon administration to a subject.

In some embodiments, a pharmaceutical composition comprising an immunogenic complex disclosed herein reduces rate of asymptomatic infection by one or more strains (variants) of SARS-CoV-2 upon administration to a subject.

Some embodiments provide for a method of immunizing a subject against one or more strains (variants) of SARS-CoV-2 infection comprising administering to the subject an immunologically effective amount of an immunogenic complex described herein. Some embodiments provide for a method of immunizing a subject against one or more strains (variants) of SARS-CoV-2 infection comprising administering to the subject an immunologically effective amount of an immunogenic composition described herein. Some embodiments provide for a method of immunizing a subject against one or more strains (variants) of SARS-CoV-2 infection comprising administering to the subject an immunologically effective amount of a vaccine composition described herein. Some embodiments provide for a method of immunizing a subject against one or more strains (variants) of SARS-CoV-2 infection comprising administering to the subject an immunologically effective amount of a pharmaceutical composition described herein.

Some embodiments provide an immunogenic complex described herein for use in the treatment or prevention of COVID-19 associated with or induced by one of more strains (variants) of SARS-CoV-2. Some embodiments provide an immunogenic composition described herein (e.g., in some embodiments, a vaccine composition described herein) for use in the treatment or prevention of COVID-19 associated with or induced by one of more strains (variants) of SARS-CoV-2. Some embodiments provide a pharmaceutical composition described herein for use in the treatment or prevention of COVID-19 associated with or induced by one of more strains (variants) of SARS-CoV-2.

Some embodiments provide a use of an immunogenic complex described herein in the manufacture of a medicament for the treatment or prevention of COVID-19 associated with or induced by one or more strains (variants) of SARS-CoV-2. Some embodiments provide a use of an immunogenic composition described herein (e.g., in some embodiments, a vaccine composition described herein) in the manufacture of a medicament for the treatment or prevention of COVID-19 associated with or induced by one or more strains (variants) of SARS-CoV-2. Some embodiments provide a use of a pharmaceutical composition described herein in the manufacture of a medicament for the treatment or prevention of COVID-19 associated with or induced by one or more strains (variants) of SARS-CoV-2.

The characteristics of methods of treatment or prevention disclosed herein are applicable to an immunogenic complex, immunogenic composition (e.g., in some embodiments, a vaccine composition), or pharmaceutical composition for use or a use of an immunogenic complex, immunogenic composition (e.g., in some embodiments, a vaccine composition), or pharmaceutical composition in the manufacture of a medicament.

Dosing

In some embodiments, administration of a composition described herein (e.g., an immunogenic composition described herein, which in some embodiments is a vaccine composition described herein, or a pharmaceutical composition described herein) may involve the delivery of a single dose. In some embodiments, administration may involve an initial dose followed by one or several additional immunization doses, adequately spaced. In some embodiments, an initial dose and subsequent dose(s) may comprise administration of the same composition (e.g., an immunogenic composition described herein, which in some embodiments a vaccine composition described herein, or a pharmaceutical composition described herein). In some embodiments, an initial dose and subsequent dose(s) may comprise administration of different compositions described herein (e.g., immunogenic compositions described herein, which in some embodiments vaccine compositions described herein, or pharmaceutical compositions described herein). An immunization schedule or regimen is a program for the administration of one or more specified doses of one or more specified vaccines, by one or more specified routes of administration, at one or more specified ages of a subject.

The present disclosure provides immunization methods that involve administering at least one dose of a vaccine to an infant subject. In some embodiments, the infant subject is 18 months old or younger. In some embodiments, the infant subject is 12 months old or younger. In some embodiments, the infant subject is 6 months old or younger. In some embodiments, the infant subject has previously been infected with, or exposed to infection by SARS-CoV-2.

The present disclosure provides immunization methods that involve administering at least one dose of a vaccine to a toddler subject. In some embodiments, the toddler subject is 5 years old or younger. In some embodiments, the toddler subject is 4 years old or younger. In some embodiments, the toddler subject has previously been infected with, or exposed to infection by SARS-CoV-2.

The present disclosure provides immunization methods that involve administering at least one dose of a vaccine to a juvenile subject. In some embodiments, the juvenile subject is 18 years old or younger. In some embodiments, the juvenile subject is 15 years old or younger. In some embodiments, the juvenile subject has previously been infected with, or exposed to infection by SARS-CoV-2.

The present disclosure provides immunization methods that involve administering at least one dose of a vaccine to an adult subject. In some embodiments, the adult subject is older than about 18 years of age. In some embodiments, the adult subject is older than about 50 years of age. In some embodiments, the adult subject is older than about 65 years of age. In some embodiments, the adult subject has previously been infected with, or exposed to infection by SARS-CoV-2.

Immunization schedules of the present disclosure are provided to induce an immune response (e.g., an immunoprotective response) in a subject sufficient to reduce at least one measure selected from the group consisting of incidence, prevalence, frequency, and/or severity of at least one infection, disease, or disorder, and/or at least one surrogate marker of the infection, disease, or disorder, in a population and/or subpopulation of the subject(s). A supplemental immunization schedule is one which has this effect relative to the standard schedule which it supplements. A supplemental schedule may call for additional administrations and/or supra-immunogenic doses of the immunogenic compositions disclosed herein, found in the standard schedule, or for the administration of vaccines not part of the standard schedule. A full immunization schedule of the present invention may comprise both a standard schedule and a supplemental schedule. Exemplary sample vaccination schedules are provided for illustrative purposes. Detailed descriptions of methods to assess immunogenic response discussed herein allow one to develop alterations to the sample immunization schedules without undue experimentation.

In some embodiments of the present disclosure, a first administration of a composition described herein (e.g., an immunogenic composition described herein, which in some embodiments is a vaccine composition described herein, or a pharmaceutical composition described herein) occurs when a subject is more than about 2 weeks old, more than about 5 weeks old, more than 6 months old, more than about 1 year old, more than about 2 years old, more than about 15 years old, or more than about 18 years old.

In some embodiments, a first administration of a composition described herein (e.g., an immunogenic composition described herein, which in some embodiments is a vaccine composition described herein, or a pharmaceutical composition described herein) occurs when a subject is about two months old. In some embodiments, a second administration of a composition described herein (e.g., an immunogenic composition described herein, which in some embodiments is a vaccine composition described herein, or a pharmaceutical composition described herein) occurs when a subject is about four months old. In some embodiments, a third administration of a composition described herein (e.g., an immunogenic composition described herein, which in some embodiments is a vaccine composition described herein, or a pharmaceutical composition described herein) occurs when a subject is about six months old. In some embodiments, a fourth administration of a composition described herein (e.g., an immunogenic composition described herein, which in some embodiments is a vaccine composition described herein, or a pharmaceutical composition described herein) occurs when a subject is between about twelve months old and about fifteen months old.

In some embodiments of the present disclosure, a first administration of a composition described herein (e.g., an immunogenic composition described herein, which in some embodiments is a vaccine composition described herein, or a pharmaceutical composition described herein) occurs when a subject is more than about 18 years old, more than about 50 years old, more than about 55 years old, more than about 60 years old, more than about 65 years old, or more than about 70 years old.

In some embodiments of the disclosure, a single administration of a composition described herein (e.g., an immunogenic composition described herein, which in some embodiments is a vaccine composition described herein, or a pharmaceutical composition described herein) is employed. It is possible that the purposes of the present invention can be served with a single administration, especially when one or more utilized vaccine polypeptide(s), polysaccharide(s) and/or immunogenic complex(es) or combinations thereof is/are strong, and in such a situation a single dose schedule is sufficient to induce a lasting immune-protective response.

In certain embodiments, it is desirable to administer two or more doses of a composition described herein (e.g., an immunogenic composition described herein, which in some embodiments is a vaccine composition described herein, or a pharmaceutical composition described herein), for greater immune-protective efficacy and coverage. Thus, in some embodiments, a number of doses is at least two, at least three or more doses. There is no set maximum number of doses, however it is good clinical practice not to immunize more often than necessary to achieve the desired effect.

Without being bound by theory, a first dose of vaccine administered according to the disclosure may be considered a “priming” dose. In certain embodiments, more than one dose is included in an immunization schedule. In such a scenario, a subsequent dose may be considered a “boosting” dose.

A priming dose may be administered to a naïve subject (a subject who has never previously received a vaccine). In some embodiments, a priming dose may be administered to a subject who has previously received a vaccine at least five or more years previous to administration of an initial vaccine dose according to the invention. In other embodiments, a priming dose may be administered to a subject who has previously received a vaccine at least twenty or more years previous to administration of a priming vaccine according to the disclosure.

When an immunization schedule calls for two or more separate doses, the interval between doses is considered. The interval between two successive doses may be the same throughout an immunization schedule, or it may change as the subject ages. In immunization schedules of the present invention, once a first vaccine dose has been administered, there is a first interval before administration of a subsequent dose. A first interval is generally at least about 2 weeks, 1 month, 6 weeks, 2 months, 3 months, 6 months, 9 months, 12 months, or longer. Where more than one subsequent dose(s) are administered, second (or higher) intervals may be provided between such subsequent doses. In some embodiments, all intervals between subsequent doses are of the same length; in other embodiments, second intervals may vary in length. In some embodiments, the interval between subsequent doses may be at least about 12 months, at least about 15 months, at least about 18 months, at least about 21 months or at least about 2 years. In certain embodiments, the interval between doses may be up to 3 years, up to about 4 years, or up to about 5 years or 10 years or more. In certain embodiments, intervals between subsequent doses may decrease as the subject ages.

It will be appreciated by those skilled in the art that a variety of possible combinations and sub-combinations of the various conditions of timing of the first administration, shortest interval, largest interval and total number of administrations (in absolute terms, or within a stated period) exist, and all of these combinations and sub-combinations should be considered to be within the contemplation of the present disclosure though not explicitly enumerated here.

Assays for Determining Immune Response

In some embodiments, a method of assessing the immunogenicity of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by opsonophagocytic killing assay (OPK, OPA), plaque reduction neutralization test (PRNT), agglutination, motility, cytotoxicity, or adherence; and in vivo assays in animal models of COVID-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the potency of an immunogenic composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), internalization, activity neutralization, agglutination, motility, cytotoxicity, or adherence; and in vivo assays in animal models of COVID-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the immunogenic composition and not comprise an antigenic polypeptide present in the immunogenic composition. In some embodiments, a control composition may comprise an antigenic polypeptide present in the immunogenic composition and not comprise an antigenic polysaccharide present in the immunogenic composition. In some embodiments, a control composition may comprise an adjuvant present in the immunogenic composition, and not comprise an antigenic polysaccharide and/or an immunogenic polypeptide present in the immunogenic composition.

In some embodiments, a method of assessing the immunogenicity of a vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), agglutination, motility, cytotoxicity, or adherence; and in vivo assays in animal models of COVID-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the vaccine composition and not comprise an antigenic polypeptide present in the vaccine composition. In some embodiments, a control composition may comprise an antigenic polypeptide present in the vaccine composition and not comprise an antigenic polysaccharide present in the vaccine composition. In some embodiments, a control composition may comprise an adjuvant present in the vaccine composition, and not comprise an antigenic polysaccharide and/or an immunogenic polypeptide present in the vaccine composition.

In some embodiments, a method of assessing the potency of a vaccine composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), agglutination, motility, cytotoxicity, or adherence; and in vivo assays in animal models of COVID-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the vaccine composition and not comprise an antigenic polypeptide present in the vaccine composition. In some embodiments, a control composition may comprise an antigenic polypeptide present in the vaccine composition and not comprise an antigenic polysaccharide present in the vaccine composition. In some embodiments, a control composition may comprise an adjuvant present in the vaccine composition, and not comprise an antigenic polysaccharide and/or an immunogenic polypeptide present in the vaccine composition.

In some embodiments, a method of assessing the immunogenicity of a pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), agglutination, motility, cytotoxicity, or adherence; and in vivo assays in animal models of COVID-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the pharmaceutical composition and not comprise an antigenic polypeptide present in the pharmaceutical composition. In some embodiments, a control composition may comprise an antigenic polypeptide present in the pharmaceutical composition and not comprise an antigenic polysaccharide present in the pharmaceutical composition. In some embodiments, a control composition may comprise an adjuvant present in the pharmaceutical composition, and not comprise an antigenic polysaccharide and/or an immunogenic polypeptide present in the pharmaceutical composition.

In some embodiments, a method of assessing the potency of a pharmaceutical composition described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), agglutination, motility, cytotoxicity, or adherence; and in vivo assays in animal models of COVIS-19. Parameters of in vivo assays include viral clearance, reduction of mortality, and passive and active protection following challenge with the one or more strains (variants) of SARS-CoV-2 that are the targets of the immunogenic composition. In some embodiments, the immune response is compared to a control composition. In some embodiments, a control composition may comprise an antigenic polysaccharide present in the pharmaceutical composition and not comprise an antigenic polypeptide present in the pharmaceutical composition. In some embodiments, a control composition may comprise an antigenic polypeptide present in the pharmaceutical composition and not comprise an antigenic polysaccharide present in the pharmaceutical composition. In some embodiments, a control composition may comprise an adjuvant present in the pharmaceutical composition, and not comprise an antigenic polysaccharide and/or an immunogenic polypeptide present in the pharmaceutical composition.

In some embodiments, a method of assessing the immunogenicity and/or potency of an immunogenic complex comprises evaluating an immune response to immunogenic or vaccine compositions comprising one or more immunogenic complexes. In some embodiments, the method of assessing the immunogenicity and/or potency of an immunogenic complex described herein comprises evaluating, measuring, and/or comparing an immune response using one or more in vitro bioassays, including B cell and T cell responses such as antibody levels by ELISA, multiplex ELISA, MSD, Luminex, flow cytometry, T_{H}1/T_{H}17 cell response, cytokine level measurement and functional antibody levels as measured by OPK (OPA), plaque reduction neutralization test (PRNT), agglutination, motility, cytotoxicity, or adherence; and in vivo assays in animal models of COVID-19. Parameters of in vivo assays include viral clearance, reduction in mortality, and passive and active protection following challenge with the one or more strains (variants) of SARS-CoV-2 that are the targets of the immunogenic composition.

Generally speaking, it may be desirable to assess humoral responses, cellular responses, and/or interactions between the two. Where humoral responses are being assessed, antibody titers and/or types (e.g., total IgG, IgG1, IgG2, IgM, IgA, etc.) to specific pathogen polysaccharides or polypeptides (either serotype-specific or conserved across two or more serotypes) may be determined, for example before and/or after administration of an initial or a boosting dose of vaccine (and/or as compared with antibody levels in the absence of antigenic stimulation). Cellular responses may be assessed by monitoring reactions such as delayed type hypersensitivity responses, etc. to the carrier protein. Cellular responses can also be measured directly by evaluating the response of peripheral blood mononuclear cells (PBMCs) monocytes to stimulation with the antigens of interest. Precursor and memory B cell populations may be assessed in enzyme linked immunospot (ELISpot) assays directed against specific pathogen polysaccharides or polypeptides.

Any of a variety of assays may be employed to detect levels and/or activity of antibodies in subject sera. Suitable assays include, for example, ligand binding assays, such as radioimmunoassay (RIAs), ELISAs, and multiplex assays (Luminex, Bioplex, MSD); functional assays, such as opsonophagocytic assays (OPK, OPA), plaque reduction neutralization test (PRNT), or internalization assays; and in vivo assays in animal models of COVID-19. Parameters of in vivo assays include viral clearance, reduction in mortality, and passive and active protection following challenge with the one or more strains (variants) of SARS-CoV-2 that are the targets of the immunogenic composition.

The RIA method detects specific antibodies through incubation of sera with radiolabeled polysaccharides or polypeptides in suspension (e.g., Schiffiman et al, 1980). The antigen-antibody complexes are then precipitated with ammonium sulfate and the radiolabeled pellets assayed for counts per minute (cpm).

In the ELISA detection method, specific antibodies from the sera of vaccinated subjects are quantitated by incubation with polysaccharides or polypeptides (either serotype-specific or conserved across two or more serotypes) which have been adsorbed to a solid support (e.g., Koskela and Leinonen (1981); Kojima et al, 1990; Concepcion and Frasch, 2001). The bound antibody is detected using enzyme-conjugated secondary detection antibodies. The ELISA also allows isotyping and subclassing of the immune response (i.e., IgM vs. IgG or IgG1 vs. IgG2) by using isotype- or subclass-specific secondary antibodies and can be adapted to evaluate the avidity of the antibodies (Anttila et al, 1998; Romero-Steiner et al, 2005). Multiplex assays (e.g., Luminex) facilitate simultaneous detection of antibodies to multiple antigens. Capsular polysaccharide(s) or polypeptides are conjugated to spectrally distinct microspheres that are mixed and incubated with serum. The antibodies bound to the polysaccharides or polypeptides on the coated microspheres are detected using a secondary antibody (e.g., R-Phycoerythrin-conjugated goat anti-human IgG).

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against pneumonia, mortality, or other endpoint is determined (Stack et al. 1998; Saeland et al. 2000).

In some embodiments, efficacy of vaccination may be determined by assaying one or more cytokine levels by stimulating T cells from a subject after vaccination. The one or more cytokine levels may be compared to the one or more cytokine levels in the same subject before vaccination. Increased levels of the one or more cytokine, such as a 1.5-fold, 2-fold, 5-fold, 10-fold, 20-fold, 50-fold or 100-fold or more increase over pre-immunization cytokine levels, would indicate an increased response to the vaccine. In some embodiments, the one or more cytokines are selected from GM-CSP; IL-1α; IL-1β; IL-2; IL-3; IL-4; IL-5; IL-6; IL-7; IL-8; IL-10; IL-12; IL-17A, IL-17F or other members of the IL-17 family; IL-22; IL-23; IFN-α; IFN-β; IFN-γ; MIP-1α; MIP-1β; TGF-β; TNFα, or TNF-β. In a non-limiting example, efficacy of vaccination may be determined by assaying IL-17 levels (particularly IL-17A) by stimulating T cells from a subject after vaccination. The IL-17 levels may be compared to IL-17 levels in the same subject before vaccination. Increased IL-17 (e.g., IL-17A) levels, such as a 1.5 fold, 2-fold, 5-fold, 10-fold, 20-fold, 50-fold or 100-fold or more increase, would indicate an increased response to the vaccine.

In some embodiments, one may assay neutrophils in the presence of T cells or antibodies from the patient for viral killing. Increased viral killing, such as a 1.5-fold, 2-fold, 5-fold, 10-fold, 20-fold, 50-fold or 100-fold or more increase, would indicate an increased response to the vaccine. For example, one may measure T_{H}17 cell activation, where increased T_{H}17 cell activation, such as a 1.5 fold, 2-fold, 5-fold, 10-fold, 20-fold, 50-fold or 100-fold or more increase, correlates with an increased response to the vaccine. In another non-limiting example, one may measure T_{H}1 cell activation, where increased T_{H}1 cell activation, such as a 1.5 fold, 2-fold, 5-fold, 10-fold, 20-fold, 50-fold or 100-fold or more increase, correlates with an increased response to the vaccine. One may also measure levels of an antibody specific to the vaccine, where increased levels of the specific antibody, such as a 1.5 fold, 2-fold, 5-fold, 10-fold, 20-fold, 50-fold or 100-fold or more increase, are correlated with increased vaccine efficacy. In certain embodiments, two or more of these assays are used. For example, one may measure IL-17 levels and the levels of vaccine-specific antibody. Alternatively, one may follow epidemiological markers such as incidence of, severity of, or duration of viral infection in vaccinated individuals compared to unvaccinated individuals.

Vaccine efficacy may also be assayed in various model systems such as the mouse challenge model. For instance, BALB/c or C57BL/6 strains of mice may be used. After administering the test vaccine to a subject (as a single dose or multiple doses), the experimenter administers a challenge dose of SARS-CoV-2. In some cases, a challenge dose administered intranasally or intratracheally is sufficient to cause SARS-CoV-2 infection and/or a high rate of lethality in unvaccinated animals. One can then measure the reduction in infection and/or the reduction in lethality in vaccinated animals.

Certain in vivo model systems can be used to evaluate the protection afforded by serum antibodies induced by vaccines of the present invention. In such passive protection systems, mice or rats are challenged with the pathogen plus diluted sera, and the endpoint titer of the sera which provides protection against bacteremia, colonization of organs or tissues, or mortality is determined (Stack et al. 1998; Saeland et al. 2000).

Kits

The present disclosure also provides for kits for producing an immunogenic complex as disclosed herein which is useful for an investigator to tailor an immunogenic complex with their preferred antigens, e.g., for research purposes to assess the effect of an antigen, or a combination of antigens on immune response. Such kits can be prepared from readily available materials and reagents. For example, such kits can comprise any one or more of the following materials: a container comprising a polysaccharide cross-linked with a plurality of first affinity molecules; a container comprising a complementary affinity molecule which associates with the first affinity molecule, wherein the complementary affinity molecule associates with an antigen or carrier protein; a container comprising an antigen; a container comprising a carrier protein; a container comprising an antigen associated with a complementary affinity molecule; a container comprising a carrier protein associated with a complementary affinity molecule.

In another embodiment, the kit comprises a container comprising a polysaccharide; a container comprising a plurality of first affinity molecules; and a container comprising a cross-linking reagent for cross-linking the first affinity molecules to the polysaccharide, for example, but not limited to, CDAP (1-cyano-4-dimethylaminopyridinium tetrafluoroborate), and EDC (1-Ethyl-3-[3-dimethylaminopropyl] carbodiimide hydrochloride).

In another embodiment, the kit comprises a container comprising an antigen or carrier protein, and a container comprising a complementary affinity molecule which associates with a first affinity molecule. In some embodiments, the kit further comprises a means to attach the complementary affinity molecule to the antigen or carrier protein, where the means can be by a cross-linking reagent or by some intermediary fusion protein.

In some embodiments, the kit can comprise at least one co-stimulation factor which can be added to the polymer. In some embodiments, the kit comprises a cross-linking reagent, for example, but not limited to, CDAP (1-cyano-4-dimethylaminopyridinium tetrafluoroborate); EDC (1-Ethyl-3-[3-dimethylaminopropyl] carbodiimide hydrochloride); sodium cyanoborohydride; cyanogen bromide; and ammonium bicarbonate/iodoacetic acid, for linking the co-factor to the polymer.

A variety of kits and components can be prepared for use in the methods described herein, depending upon the intended use of the kit, the particular target antigen and the needs of the user.

### Exemplary Embodiments

Exemplary embodiments as described below are also within the scope of the present disclosure:

1. A vaccine comprising one or more species of immunogenic complexes, wherein each immunogenic complex comprises:

(a) a biotinylated polysaccharide antigen; and

(b) a fusion protein comprising:
(i) a biotin-binding moiety; and
(ii) at least one polypeptide antigen of SARS-CoV-2;

wherein the biotinylated polysaccharide antigen is non-covalently associated with the biotin-binding moiety of the fusion protein to form an immunogenic complex.

2. The vaccine of embodiment 1, wherein the fusion protein comprises at least one of:

(a) a Spike (S) polypeptide antigen or antigenic fragment thereof;

(b) an Envelope (E) polypeptide antigen or antigenic fragment thereof;

(c) a Membrane (M) polypeptide antigen or antigenic fragment thereof; and

(d) a Nucleocapsid (N) polypeptide or antigenic fragment thereof.

3. The vaccine of embodiment 2, wherein the fusion protein comprises at least one of:

(a) a Spike (S) polypeptide antigen or antigenic fragment thereof, and

(b) a Membrane (M) polypeptide antigen or antigenic fragment thereof.

4. The vaccine of embodiment 3, wherein the fusion protein comprises the Receptor Binding Domain (RBD) of a Spike (S) polypeptide antigen or antigenic fragment thereof.
5. The vaccine of embodiment 3, wherein the fusion protein comprises one or more Extra-Cellular Domains (ECDs) of a Membrane (M) polypeptide antigen or antigenic fragment thereof.
6. The vaccine of embodiment 3, wherein the fusion protein comprises:

(a) the Receptor Binding Domain (RBD) of a Spike (S) polypeptide antigen or antigenic fragment thereof; and

(b) one or more Extra-Cellular Domains (ECDs) of a Membrane (M) polypeptide antigen or antigenic fragment thereof.

7. The vaccine of any one of the preceding embodiments, wherein the one or more species of immunogenic complexes comprise polypeptide antigen(s) of one strain (variant) of SARS-CoV-2.
8. The vaccine of any one of the preceding embodiments, wherein the one or more species of immunogenic complexes comprise polypeptide antigen(s) of multiple strains (variants) of SARS-CoV-2.
9. The vaccine of any one of the preceding embodiments, comprising one species of immunogenic complexes, wherein the species comprises the same fusion protein.
10. The vaccine of any one of the preceding embodiments, comprising a plurality of different species of immunogenic complexes, wherein the plurality of different species comprises a plurality of different fusion proteins.
11. The vaccine of embodiment 4 or embodiment 6, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, 18, 21, 24, 27, 30, 33, 36, 39 or 42, or an antigenic fragment thereof.
12. The vaccine of embodiment 4 or embodiment 6, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, or 18, or an antigenic fragment thereof.
13. The vaccine of embodiment 4 or embodiment 6, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:33, 36, 39 or 42, or an antigenic fragment thereof.
14. The vaccine of embodiment 11, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, 20, 23, 26, 29, 32, 35, 38, 41 or 44, or an antigenic fragment thereof.
15. The vaccine of embodiment 12, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, or 20, or an antigenic fragment thereof.
16. The vaccine of embodiment 13, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:35, 38, 41 or 44, or an antigenic fragment thereof.
17. The vaccine of any one of the preceding embodiments, wherein the biotinylated polysaccharide antigen comprises a polysaccharide of Streptococcus pneumoniae.
18. The vaccine of embodiment 17, wherein the biotinylated polysaccharide antigen comprises a polysaccharide of Streptococcus pneumoniae selected from serotypes 1, 9N, and 19A.
19. The vaccine of embodiment 18, wherein the biotinylated polysaccharide antigen comprises a polysaccharide of Streptococcus pneumoniae serotype 1 (PS1).
20. The vaccine of any one of the preceding embodiments, wherein the biotin-binding moiety is a polypeptide comprising (i) an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:1 or a biotin-binding fragment thereof; or (ii) a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO:2, or a biotin-binding fragment thereof.
21. An immunogenic complex comprising:

(a) a biotinylated polysaccharide antigen; and

(b) a fusion protein comprising:
(i) a biotin-binding moiety; and
(ii) at least one polypeptide antigen of SARS-CoV-2;

wherein the biotinylated polysaccharide antigen is non-covalently associated with the biotin-binding moiety of the fusion protein.

22. The immunogenic complex of embodiment 21, wherein the fusion protein comprises at least one of:

(a) a Spike (S) polypeptide antigen or antigenic fragment thereof;

(b) an Envelope (E) polypeptide antigen or antigenic fragment thereof;

(c) a Membrane (M) polypeptide antigen or antigenic fragment thereof; and

(d) a Nucleocapsid (N) polypeptide or antigenic fragment thereof.

23. The immunogenic complex of embodiment 22, wherein the fusion protein comprises at least one of:

(a) a Spike (S) polypeptide antigen or antigenic fragment thereof, and

(b) a Membrane (M) polypeptide antigen or antigenic fragment thereof.

24. The immunogenic complex of embodiment 23, wherein the fusion protein comprises the Receptor Binding Domain (RBD) of a Spike (S) polypeptide antigen or antigenic fragment thereof.
25. The immunogenic complex of embodiment 23, wherein the fusion protein comprises one or more Extra-Cellular Domains (ECDs) of a Membrane (M) polypeptide antigen or antigenic fragment thereof.
26. The immunogenic complex of embodiment 23, wherein the fusion protein comprises:

(a) the Receptor Binding Domain (RBD) of a Spike (S) polypeptide antigen or antigenic fragment thereof; and

(b) one or more Extra-Cellular Domains (ECDs) of a Membrane (M) polypeptide antigen or antigenic fragment thereof.

27. The immunogenic complex of any one of the preceding embodiments, comprising one polypeptide antigen.
28. The immunogenic complex of any one of the preceding embodiments, comprising more than one polypeptide antigen.
29. The immunogenic complex of any one of the preceding embodiments, comprising polypeptide antigen(s) of one strain (variant) of SARS-CoV-2.
30. The immunogenic complex of any one of the preceding embodiments, comprising polypeptide antigen(s) of multiple strains (variants) of SARS-CoV-2.
31. The immunogenic complex of embodiment 24 or embodiment 26, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or identical to any of SEQ ID NO:3, 6, 9, 12, 15, 18, 21, 24, 27, 30, 33, 36, 39 or 42, or an antigenic fragment thereof.
32. The immunogenic complex of embodiment 24 or embodiment 26, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, or 18, an antigenic fragment thereof.
33. The immunogenic complex of embodiment 24 or embodiment 26, wherein the polypeptide antigen is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:33, 36, 39 or 42, or an antigenic fragment thereof.
34. The immunogenic complex of embodiment 31, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, 20, 23, 26, 29, 32, 35, 38, 41 or 44, or an antigenic fragment thereof.
35. The immunogenic complex of embodiment 32, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, 20, or an antigenic fragment thereof.
36. The immunogenic complex of embodiment 33, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO: 35, 38, 41 or 44, or an antigenic fragment thereof.
37. The immunogenic complex of any one of the preceding embodiments, wherein the biotinylated polysaccharide antigen comprises a polysaccharide of Streptococcus pneumoniae.
38. The immunogenic complex of embodiment 31, wherein the biotinylated polysaccharide antigen comprises a polysaccharide of Streptococcus pneumoniae selected from serotypes 1, 9N, and 19A.
39. The immunogenic complex of embodiment 32, wherein the biotinylated polysaccharide antigen comprises a polysaccharide of Streptococcus pneumoniae serotype 1 (PS1).
40. The immunogenic complex of any one of the preceding embodiments, wherein the biotin-binding moiety is a polypeptide comprising (i) an amino acid sequence at least 90%, at least 91%, at least 92%, at least 93%, at least 94%, at least 95%, at least 96%, at least 97%, at least 98%, at least 99%, or at least 99.5% identical to SEQ ID NO:1 or a biotin-binding fragment thereof; or (ii) a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO:2, or a biotin-binding fragment thereof.
41. A vaccine composition comprising one or more immunogenic complexes of any one of embodiments 21-40.
42. A pharmaceutical composition comprising the vaccine of any one of embodiments 1-20 and 41, and a pharmaceutically acceptable carrier.
43. A pharmaceutical composition comprising an immunogenic complex of any one of embodiments 21-40, and a pharmaceutically acceptable carrier.
44. The pharmaceutical composition of embodiment 42 or embodiment 43, further comprising one or more adjuvants.
45. The pharmaceutical composition of embodiment 44, wherein the one or more adjuvants are or comprise a co-stimulation factor.
46. The pharmaceutical composition of embodiment 44 or embodiment 45, wherein the one or more adjuvants are selected from the group consisting of aluminum phosphate, aluminum hydroxide, and phosphated aluminum hydroxide.
47. The pharmaceutical composition of any one of embodiments 44-46, wherein the one or more adjuvants are or comprise aluminum phosphate.
48. The pharmaceutical composition of any one of embodiments 42-47, wherein the pharmaceutical composition is formulated for injection.
49. The pharmaceutical composition of any one of embodiments 42-48, wherein the pharmaceutical composition is characterized in that upon administration to a subject, the pharmaceutical composition induces an immune response.
50. The pharmaceutical composition of embodiment 49, wherein the immune response comprises an antibody and/or B cell response.
51. The pharmaceutical composition of embodiment 50, wherein the antibody and/or B cell response comprises a memory B cell response.
52. The pharmaceutical composition of any one of embodiments 49-51, wherein the immune response comprises a CD4+ T cell response (e.g., T_{H}1, T_{H}2, or T_{H}17 response); a CD8+ T cell response; a CD4+ and CD8+ T cell response; or a CD4−/CD8− T cell response.
53. The pharmaceutical composition of embodiment 52, wherein the T cell response comprises a memory T cell response.
54. The pharmaceutical composition of any one of embodiments 49-53, wherein the immune response comprises (i) an antibody or B cell response and (ii) a T cell response.
55. The pharmaceutical composition of any one of embodiments 49-54, wherein the immune response is to (i) at least one polysaccharide antigen of the vaccine or immunogenic complex, and/or (ii) at least one polypeptide antigen of the vaccine or immunogenic complex.
56. The pharmaceutical composition of any one of embodiments 49-55, wherein the immune response comprises (i) an antibody or B cell response to at least one polysaccharide antigen of the vaccine or immunogenic complex, and (ii) a CD4+ T cell response (e.g., T_{H}1, T_{H}2, or T_{H}17 response), a CD8+ T cell response, a CD4+ and CD8+ T cell response, or a CD4−/CD8− T cell response to at least one polypeptide antigen of the vaccine or immunogenic complex.
57. The pharmaceutical composition of any one of embodiments 49-56, wherein the immune response comprises (i) an antibody or B cell response to at least one polysaccharide antigen of the vaccine or immunogenic complex, and (ii) an antibody or B cell response to at least one polypeptide antigen of the vaccine or immunogenic complex.
58. The pharmaceutical composition of any one of embodiments 49-57, wherein the immune response comprises (i) an antibody or B cell response to at least one polysaccharide antigen of the vaccine or immunogenic complex, and (ii) an antibody or B cell response; and a CD4+ T cell response (including T_{H}1, T_{H}2, or T_{H}17 response), a CD8+ T cell response, a CD4+ and CD8+ T cell response, or a CD4−/CD8− T cell response to at least one polypeptide antigen of the vaccine or immunogenic complex.
59. The pharmaceutical composition of any one of embodiments 49-58, wherein the pharmaceutical composition is characterized in that upon administration to a subject, the pharmaceutical composition induces neutralizing antibodies against one or more strains (variants) of SARS-CoV-2.
60. The pharmaceutical composition of any one of embodiments 49-59, wherein the pharmaceutical composition is characterized in that upon administration to a subject, the pharmaceutical composition reduces or inhibits transmission of one or more strains (variants) of SARS-CoV-2 from the subject to another subject.
61. The pharmaceutical composition of any one of embodiments 49-59, wherein the pharmaceutical composition is characterized in that upon administration to a subject, the pharmaceutical composition reduces or inhibits replication, and/or reduces viral load, of one or more strains (variants) of SARS-CoV-2.
62. The pharmaceutical composition of any one of embodiments 49-59, wherein the pharmaceutical composition is characterized in that upon administration to a subject, the pharmaceutical composition inhibits, or reduces the rate of occurrence of, COVID-19 associated with or induced by one or more strains (variants) of SARS-CoV-2.
63. The pharmaceutical composition of any one of embodiments 49-59, wherein the pharmaceutical composition is characterized in that upon administration to a subject, the pharmaceutical composition reduces the severity of COVID-19 associated with or induced by one or more strains (variants) of SARS-CoV-2.
64. The pharmaceutical composition of any one of embodiments 49-59, wherein the pharmaceutical composition is characterized in that upon administration to a subject, the pharmaceutical composition inhibits, or reduces the rate of occurrence of, pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2.
65. The pharmaceutical composition of any one of embodiments 49-59, wherein the pharmaceutical composition is characterized in that upon administration to a subject, the pharmaceutical composition reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2.
66. The pharmaceutical composition of any one of embodiments 49-59, wherein the pharmaceutical composition is characterized in that upon administration to a subject, the pharmaceutical composition inhibits, or reduces the rate of, shedding of one or more strains (variants) of SARS-CoV-2.
67. The pharmaceutical composition of any one of embodiments 49-59, wherein the pharmaceutical composition is characterized in that upon administration to a subject, the pharmaceutical composition inhibits, or reduces the rate of, asymptomatic infection by one or more strains (variants) of SARS-CoV-2.
68. A method of making a vaccine, comprising non-covalently complexing a plurality of biotinylated polysaccharide antigens with a plurality of fusion proteins, wherein each fusion protein comprises at least one polypeptide antigen of SARS-CoV-2 selected from:

(a) a Spike (S) polypeptide antigen or antigenic fragment thereof;

(b) an Envelope (E) polypeptide antigen or antigenic fragment thereof;

(c) a Membrane (M) polypeptide antigen or antigenic fragment thereof; and

(d) a Nucleocapsid (N) polypeptide or antigenic fragment thereof.

69. The method of embodiment 68, wherein the plurality of biotinylated polysaccharide antigens comprises polysaccharides of Streptococcus pneumoniae serotype 1.
70. A method of immunizing a subject against one or more strains (variants) of SARS-CoV-2 comprising administering to the subject an immunologically effective amount of the vaccine of any one of embodiments 1-20 and 41.
71. A method of immunizing a subject against one or more strains (variants) of SARS-CoV-2 comprising administering to the subject an immunologically effective amount of the immunogenic complex of any one of embodiments 21-40.
72. A method of immunizing a subject against one or more strains (variants) of SARS-CoV-2 comprising administering to the subject an immunologically effective amount of the pharmaceutical composition of any one of embodiments 42-67.
73. The method of any one of embodiments 70-72, wherein the vaccine, immunogenic composition, or pharmaceutical composition induces an immune response.
74. The method of embodiment 73, wherein the immune response comprises an antibody or B cell response.
75. The method of embodiment 74, wherein the antibody or B cell response comprises a memory B cell response.
76. The method of embodiment 73, wherein the immune response comprises a CD4+ T cell response (e.g., T_{H}1, T_{H}2, or T_{H}17 response), a CD8+ T cell response, a CD4+ and CD8+ T cell response, or a CD4−/CD8− T cell response.
77. The method of embodiment 76, wherein the T cell response comprises a memory T cell response.
78. The method of embodiment 73, wherein the immune response comprises (i) an antibody or B cell response, and (ii) a T cell response.
79. The method of embodiment 78, wherein the immune response comprises (i) an antibody or B cell response, and (ii) a CD4+ T cell response (e.g., T_{H}1, T_{H}2, or T_{H}17 response), a CD8+ T cell response, a CD4+ and CD8+ T cell response, or a CD4−/CD8− T cell response.
80. The method of any one of embodiments 73-79, wherein the immune response is to at least one polypeptide of a fusion protein.
81. The method of any one of embodiments 73-80, wherein the vaccine induces neutralizing antibodies against one or more strains (variants) of SARS-CoV-2.
82. The method of any one of embodiments 73-81, wherein the vaccine reduces or inhibits transmission of one or more strains (variants) of SARS-CoV-2 from the subject to another subject.
83. The method of any one of embodiments 73-81, wherein the vaccine reduces or inhibits replication, and/or reduces viral load, of one or more strains (variants) of SARS-CoV-2.
84. The method of any one of embodiments 73-81, wherein the vaccine inhibits, or reduces the rate of occurrence of, COVID-19 associated with or induced by one or more strains (variants) of SARS-CoV-2.
85. The method of any one of embodiments 73-81, wherein the vaccine reduces the severity of COVID-19 associated with or induced by one or more strains (variants) of SARS-CoV-2.
86. The method of any one of embodiments 73-81, wherein the vaccine inhibits, or reduces the rate of occurrence of, pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2.
87. The method of any one of embodiments 73-81, wherein the vaccine reduces the severity of pneumonia, organ damage, upper respiratory symptoms, gastro-intestinal symptoms, neurological symptoms, myocarditis, inflammation, fever, chills, fatigue, headache, nausea, muscle or body ache, shortness of breath or difficulty breathing, loss of sense of smell (hyposmia, anosmia), loss of sense of taste (hypogeusia, ageusia), multi-inflammatory syndrome of children or adults (MIS-C, MIS-A), Long COVID, and/or other symptoms associated with or induced by one or more strains (variants) of SARS-CoV-2.
88. The method of any one of embodiments 73-81, wherein the vaccine inhibits, or reduces the rate of, shedding of one or more strains (variants) of SARS-CoV-2.
89. The method of any one of embodiments 73-81, wherein the vaccine inhibits, or reduces the rate of, asymptomatic infection by one or more strains (variants) of SARS-CoV-2.
90. The method of any one of embodiments 73-81, wherein the subject is immunized against one or more strains (variants) of SARS-CoV-2 with one dose of a vaccine.
91. The method of any one of embodiments 73-81, wherein the subject is immunized against one or more strains (variants) of SARS-CoV-2 with two doses of a vaccine.
92. The method of any one of embodiments 73-81, wherein the subject is immunized against one or more strains (variants) of SARS-CoV-2 with three doses of a vaccine.
93. The method of any one of embodiments 73-81, wherein the subject is immunized against one or more strains (variants) of SARS-CoV-2 with periodic doses of a vaccine.
94. The method of embodiment 93, wherein the subject is immunized against one or more strains (variants) of SARS-CoV-2 with annual doses of a vaccine.
95. The method of any one of embodiments 90-94, wherein the vaccine is administered in a regimen as a priming vaccine.
96. The method of any one of embodiments 90-94, wherein the vaccine is administered in a regimen as a booster vaccine.
97. The method of any one of embodiments 90-94, wherein the vaccine is administered in a regimen as a priming vaccine and a booster vaccine.
98. The method of any one of embodiments 90-94, wherein the regimen comprises administration of one or more additional vaccines.
99. The vaccine of any one of embodiments 1-20 and 41 for use in the treatment or prevention of COVID-19 associated with or induced by one or more strains (variants) of SARS-CoV-2.
100. A use of the vaccine of any one of embodiments 1-20 and 41 in the manufacture of a medicament for the treatment or prevention of COVID-19 associated or induced by one or more strains (variants) of SARS-CoV-2.
101. A fusion protein comprising:

(i) a biotin-binding moiety;

(ii) at least one polypeptide antigen of SARS-CoV-2.

102. The fusion protein of embodiment 101, wherein the at least one polypeptide antigen of SARS-CoV-2 is selected from:

(a) a Spike (S) polypeptide antigen or antigenic fragment thereof;

(b) an Envelope (E) polypeptide antigen or antigenic fragment thereof;

(c) a Membrane (M) polypeptide antigen or antigenic fragment thereof; and

(d) a Nucleocapsid (N) polypeptide or antigenic fragment thereof.

103. The fusion protein of embodiment 102, wherein the at least one polypeptide antigen of SARS-CoV-2 is selected from:

(a) a Spike (S) polypeptide antigen or antigenic fragment thereof, and

(b) a Membrane (M) polypeptide antigen or antigenic fragment thereof.

104. The fusion protein of any one of embodiments 101-103 comprising:

(i) a biotin-binding moiety comprising an amino acid sequence at least 80%, 85%, 90%, or 95% identical to SEQ ID NO:2 or a biotin binding portion thereof, and

(ii) a polypeptide antigen comprising an amino acid sequence at least 80%, 85%, 90%, or 95% identical to any of SEQ ID NO:3, 6, 9, 12, 15, 18, 21, 24, 27, 30, 33, 36, 39 or 42, or an antigenic fragment thereof.

105. The fusion protein of any one of embodiments 101-104 comprising:

(i) a biotin-binding moiety comprising an amino acid sequence at least 80%, at least 85%, at least 90%, or at least 95% identical to SEQ ID NO:2 or a biotin binding portion thereof; and

(ii) a polypeptide antigen comprising an amino acid sequence at least 80%, at least 85%, at least 90% or at least 95% identical to any of SEQ ID NO:3, 6, 9, 12, 15, or 18, or an antigenic fragment thereof.

106. The fusion protein of any one of embodiments 101-104 comprising:

(i) a biotin-binding moiety comprising an amino acid sequence at least 80%, at least 85%, at least 90%, or at least 95% identical to SEQ ID NO:2 or a biotin binding portion thereof; and

(ii) a polypeptide antigen comprising an amino acid sequence at least 80%, at least 85%, at least 90% or at least 95% identical to any of SEQ ID NO:33, 36, 39 or 42, or an antigenic fragment thereof.

107. The fusion protein of any one of embodiments 101-106, wherein the biotin-binding moiety is C-terminally linked at a polypeptide antigen.
108. The fusion protein of any one of embodiments 101-106, wherein the biotin-binding moiety is N-terminally linked to a polypeptide antigen.
109. The fusion protein of any one of embodiments 101-108, comprising an amino acid sequence at least 80%, at least 85%, at least 90%, at least 95%, at least 96%, at least 97%, at least 98%, at least 99%, or at least 99.5% identical to any of SEQ ID NO:5, 8, 11, 14, 17, 20, 23, 26, 29, 32, 35, 38, 41 or 44, or an antigenic fragment thereof.
110. The fusion protein of any one of embodiments 101-109, comprising an amino acid sequence at least 80%, at least 85%, at least 90%, at least 95%, at least 96%, at least 97%, at least 98%, at least 99%, or at least 99.5% identical to any of SEQ ID NO:5, 8, 11, 14, 17, or 20, or an antigenic fragment thereof.
111. The fusion protein of any one of embodiments 101-109, comprising an amino acid sequence at least 80%, at least 85%, at least 90%, at least 95%, at least 96%, at least 97%, at least 98%, at least 99%, or at least 99.5% identical to any of SEQ ID NO:35, 38, 41 or 44, or an antigenic fragment thereof.
112. The fusion protein of any one of embodiments 101-108, comprising the amino acid sequence of any of SEQ ID NO:5, 8, 11, 14, 17, 20, 23, 26, 29, 32, 35, 38, 41 or 44, or an antigenic fragment thereof.
113. The fusion protein of embodiment 112, comprising the amino acid sequence of any of SEQ ID NO:5, 8, 11, 14, 17, or 20, or an antigenic fragment thereof.
114. The fusion protein of embodiment 112, comprising the amino acid sequence of any of SEQ ID NO:35, 38, 41 or 44, or an antigenic fragment thereof.
115. A nucleic acid that comprises a nucleotide sequence encoding the fusion protein of any one of embodiments 101-114.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They is not to be construed as limiting the scope or content of the disclosure in any way.

### EXEMPLIFICATION

### Example 1: Evaluating the Multiple Antigen Presenting System (MAPS) Vaccine Platform for COVID-19 in a Nonhuman Primate (NHP) Model of Infection

Study Objective

The Multiple Antigen Presenting System (MAPS) developed by Affinivax allows disease-specific antigenic proteins and polysaccharides to be combined in a single vaccine, triggering a broad immune response. Protein antigens of interest from microorganisms such as bacteria and viruses (pathogens) are genetically fused with rhizavidin, a naturally-occurring protein with strong binding affinity to the naturally-occurring vitamin biotin, in a way that maintains immunogenic integrity of the pathogen protein(s). In parallel, polysaccharides of interest are biotinylated in a precise and reproducible way, without impacting their immunogenic properties. The rhizavidin-protein fusions are then mixed with biotinylated polysaccharides at room temperature, allowing the rhizavidin and biotin “tags” to form strong affinity bonds and create MAPS complexes that do not abrogate immunogenicity of either the polysaccharides or protein antigens, presenting them both to the immune system and eliciting a broad antibody (“B cell”) and cell-mediated (“T cell”) immune response against the pathogen.

This study evaluated the efficacy of two such candidate MAPS vaccines against SARS-CoV-2 in the cynomolgus macaque challenge model (Rockx B et al. (2020) Comparative pathogenesis of COVID-19, MERS, and SARS in a nonhuman primate model. Science 368 (6494):1012-1015).

Control and Test Articles

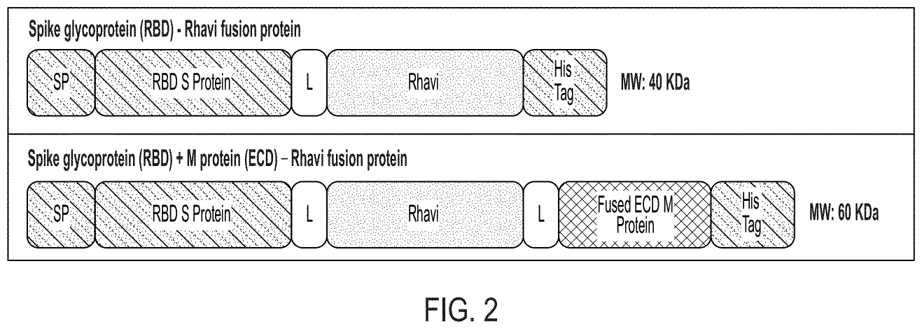

This study tested two different candidate vaccines against SARS-CoV-2, based on the Multiple Antigen Presenting System (MAPS) platform, shown graphically in FIG. 1. MAPS Vaccine #1 comprised the RBD of the Spike glycoprotein from SARS CoV-2 (ancestral Wuhan D614G strain) fused with rhizavidin (i.e., fusion protein comprising SEQ ID NO:5). MAPS Vaccine #2 comprised the RBD domain of the Spike glycoprotein from SARS CoV-2 (ancestral Wuhan D614G strain) fused with extracellular loop domains of the M protein and rhizavidin (i.e., fusion protein comprising SEQ ID NO:48). Schematics of the two fusion proteins are shown in FIG. 2. Both vaccines also comprised the well-characterized antigenic polysaccharide 1 (PS1) of Streptococcus pneumoniae.

Formulation Protocol: For each of Groups 1 and 2 (MAPS Vaccine #1 and MAPS Vaccine #2), 3.52 mL aliquots of MAPS immunogenic complexes were combined with a volume (0.180 mL) of adjuvant (ALHYDROGEL; aluminum hydroxide (AlOH) with ˜10 mg/mL aluminum content) ˜18-24 hours prior to the first and second immunizations. Once adjuvanted, test articles were kept at 4° C. with continuous rotation for at least 18 hours.

Study Design and Schedule

Eighteen (18) cynomolgus macaques of Mauritian origin were assigned to three study groups (N=6/group) as outlined in Table 2. Two groups received 1 mL doses subcutaneously of either of the two vaccine candidates, on each of Day 0 and Day 21 of the study. A placebo group received 1 mL of PBS at the two immunization time points. Serum bleeds were collected prior to and following each immunization to evaluate induction of antibody responses and cellular immunity to the vaccine antigens. Animals were challenged on study Day 42 with SARS-CoV-2 (ancestral Wuhan D614G strain) via the combination intranasal/intratracheal (IN/IT) route. Nasal swabs and bronchio-alveolar lavages (BAL) were collected throughout the post-challenge phase to assess the animals' viral load. Animals were monitored daily through post-challenge Day 14 (study Day 56), including COVID-19-specific clinical observations and respiratory assessments. Thoracic x-rays were taken on Days 4 and 10 post-challenge. On Day 56, the animals were euthanized for necropsy and tissue collection (including gross lesions, lung lobes, trachea, and heart).

TABLE 2
Study Design

| | | Immunization (SC) | | | SARS-CoV-2 | Blood | | Nasal |
|---|---|---|---|---|---|---|---|---|
| Gr | N | Material | Dose | Timepoints | Challenge | Collection | BAL | Swab |

| | | | | | | | |
|---|---|---|---|---|---|---|---|
| 1 | 6 | PBS | N/A | D0, D21 | D42 | D0, 21, 42 | D44, 46, 48, |
| | | Control | | | | | 50, 52, 54, 56 |
| 2 | 6 | Vaccine | 100 μg | D0, 21 | D42 | | |
| | | #1 | | | | | |
| 3 | 6 | Vaccine | 100 μg | D0, 21 | D42 | | |
| | | #2 | | | | | |

Study Procedures

Clinical Assessments

COVID-19 specific observations were conducted once daily during the study period. Standard cage-side observations were conducted by an animal technician once in the AM and once in the PM.

Subcutaneous Dosing with Test Articles

Test and control articles were administered via the subcutaneous route. For this procedure, the animals were anesthetized with Ketamine. The total dose was administered as a single injection between the scapulae.

Preparation and Characterization of the SARS-CoV-2 Challenge Stock

The USA-WA1/2020 isolate was expanded in Vero E6 cells from a seed stock (BEI Resources cat. no. NR-52281). The challenge stock was generated by the following method: The NR-52281 seed stock was diluted 1:200 in DMEM/2% FBS and added to Vero E6 cell ((ATCC® CRL-1586) monolayers (90-100% confluency) in T150 flasks. The cells were incubated with 5 mL diluted virus for 1 hour at 37° C., 5% CO₂ with intermittent rocking. The virus was removed and replaced with DMEM, 2% FBS. The cells were incubated at 37° C., 5% CO₂ for 5 days when CPE was observed for 80-90% of the cells. The medium was collected and centrifuged at 1500 rpm for 10 minutes at 4° C. The supernatant was maintained at 4° C. while collecting cells. Five (5) mL of DMEM, 2% FBS was added to each flask, the cells were then scraped and collected into a conical 50 mL tube. The cells were spun down at 1500 rpm for 10 minutes at 4° C. and the resulting pellet resuspended in 5 mL of DMEM, 2% FBS. The cells were freeze-thawed twice to release virus and the resulting cell lysate was combined with the supernatant. This was mixed well; 0.5 mL aliquots were prepared in cryovials and stored at ≤−70° C.

SARS-CoV-2 Challenge Procedures

Virus inoculum was prepared by serial dilution in PBS to reach the intended dose level of 10⁵ pfu in 2 mL. Administration of virus was conducted under Ketamine sedation.

Intranasal (IN) route: Using a calibrated P1000 pipettor, 0.5 mL of the viral inoculum was administered dropwise into each nostril, 1.0 mL per animal. Each anesthetized animal was held on its back on the procedure table. A technician tilted the animal's head back so that the nostrils were pointing towards the ceiling. The technician placed the tip of the syringe into the first nostril, slowly depressed the plunger, injecting the inoculum into the nasal passage, and then removed the syringe. This was repeated for the second nostril. The animal's head was tilted back for about 20 seconds and then returned to its housing unit and monitored until fully recovered.

Intratracheal (IT) route: One (1) mL of diluted virus was delivered intratracheally using a French rubber catheter/feeding tube, size 10, sterile, (cut 4″-6″ in length). The prescribed dose of inoculum was drawn into a syringe. Before inserting the syringe with the inoculum on the catheter, the technician pulled back the syringe allowing 1.5 cc of air into the syringe. This air pushed all the inoculum through the catheter. Each anesthetized animal was positioned for the procedure and its mouth was opened by an assistant. The syringe containing the inoculum was attached to a sterile French catheter or feeding tube. Once the epiglottis was visualized and the glottis opened, the small end of the feeding tube was inserted into the glottis. Once in place, the technician injected the inoculum into the trachea and then removed the catheter from the trachea. New or sterilized equipment was used for each animal. The study animal was returned to its housing unit and monitored until fully recovered.

The challenge inoculum was tested in the plaque or TCID₅₀ assay for verification of proper dose level. Remaining inoculum was also aliquoted into 2 mL cryovials and stored at ≤−70° C. for use as a positive control in the viral load assays.

Collection of Oral and Nasal Swabs

Collection of mucosal secretions was performed on each sedated animal using cotton swabs (COPAN flocked swab). The swabs were inserted into the nasal or oral cavity and rotated gently. Following collection, the swabs were placed into a collection vial (2/specimen) containing 1 mL PBS and stored at ≤−70° C. until processing for viral load testing.

Collection of Bronchio-Alveolar Lavage (BAL)

The bronchio-alveolar lavage (BAL) procedure was performed on each anesthetized animal by the “chair method”. For this procedure, one technician performed the actual BAL wash procedure, and another technician placed their hand on the animal's chest. This was to decrease or avoid any animal movement caused by possible coughing during the procedure, in order to cut down on any possible specimen contamination. The animal was placed in dorsal recumbency in the chair channel. A red rubber feeding tube was premeasured and marked before placement. The animal's head was tilted back and down below the edge of the chair channel. A red rubber feeding tube was then inserted into the animal's trachea via a laryngoscope during inspiration. The tube was placed into correct positioning and the BAL wash procedure was executed. A total of 10 mL was flushed through the tube. The volume instilled and recovered from each animal, as well as any presence of blood in the BAL samples, was recorded. Animals were monitored continuously for the first hour post-BAL collection. The collected BAL samples were placed immediately onto wet ice and processed for isolation of fluid (e.g. for viral load analysis). For this procedure, the tube was centrifuged (8 minutes at 300-400×g) at 4° C., and the supernatant removed. The following BAL aliquots were prepared and cryopreserved until viral load (VL) or other testing: 3×0.2 mL for VL testing; 3×1 mL for other assays and banking.

Blood Collection and Analysis

Whole blood was collected while animals were under anesthesia. Serum and PBMCs were prepared following standard procedures.

Necropsy Procedures

Scheduled necropsies were carried out for tissue collection following standard procedures. For viral load assays, tissues were weighed, placed into pre-labeled Sarstedt cryovials (2/sample), and snap-frozen on dry ice. Prior to testing in the viral load assay, the tissues were homogenized in 0.5 mL cold medium (DMEM/10% FBS/gentamicin) or RNA-Stat (for the PCR-based assay) for approximately 20 seconds using a hand-held tissue homogenizer. The samples were spun down to remove debris and supernatants isolated for viral load determination.

Quantitative RT-PCR Assay for SARS-CoV-2

The amounts of RNA copies per mL bodily fluid or per gram tissue was determined using a qRT-PCR assay. The qRT-PCR assay utilized primers and a probe specifically designed to amplify and bind to a conserved region of Nucleocapsid gene of coronavirus. The signal was compared to a known standard curve and calculated to give copies per mL. For the qRT-PCR assay, viral RNA was first isolated from nasal wash using the Qiagen MinElute virus spin kit (cat. no. 57704). For tissues, viral RNA was extracted with RNA-STAT 60 (Tel-test“B”)/chloroform, precipitated and resuspended in RNase-free water. To generate a control for the amplification reaction, RNA was isolated from the applicable virus stock using the same procedure. The amount of RNA was determined from an O.D. reading at 260, using the estimate that 1.0 OD at A260 equals 40 μg/mL of RNA. With the number of bases known and the average base of RNA weighing 340.5 g/mole, the number of copies was then calculated, and the control diluted accordingly. A final dilution of 10⁸ copies per 3 μL was then divided into single use aliquots of 10 μL. These were stored at −80° C. until needed. For the master mix preparation, 2.5 mL of 2× buffer containing Taq-polymerase, obtained from the TaqMan RT-PCR kit (Bioline cat #BIO-78005), was added to a 15 mL tube. From the kit, 50 μL of the RT and 100 μL of RNase inhibitor was also added. The primer pair at 2 μM concentration was then added in a volume of 1.5 mL. Lastly, 0.5 mL of water and 350 μL of the probe at a concentration of 2 μM were added and the tube vortexed. For the reactions, 45 μL of the master mix and 5 μL of the sample RNA were added to the wells of a 96-well plate. All samples were tested in triplicate.

For control curve preparation, samples of the control RNA were obtained from the −80° C. freezer. The control RNA was prepared to contain 10⁶ to 10⁷ copies per 3 μL. Eight (8) 10-fold serial dilutions of control RNA were prepared using RNase-free water by adding 5 μL of the control to 45 μL of water and repeating this for 7 dilutions. This gave a standard curve with a range of 1 to 107 copies/reaction. Duplicate samples of each dilution were prepared as described above. For amplification, the plate was placed in an Applied Biosystems 7500 Sequence detector and amplified using the following program: 48° C. for 30 minutes, 95° C. for 10 minutes followed by 40 cycles of 95° C. for 15 seconds, and 1 minute at 55° C. The number of copies of RNA per mL was calculated by extrapolating from the standard curve and multiplying by the reciprocal of 0.2 mL extraction volume. This gave a practical range of 50 to 5×10⁸ RNA copies per mL for nasal washes; for tissues, the viral loads are given per gram.

Primers/probe sequences:
2019-nCoV_N1-F:
(SEQ ID NO: 83)
5′-GAC CCC AAA ATC AGC GAA AT-3′
2019-nCoV_N1-R:
(SEQ ID NO: 84)
5′-TCT GGT TAC TGC CAG TTG AAT CTG-3′
2019-nCoV_N1-P:
(SEQ ID NO: 85)
5′-FAM-ACC CCG CAT TAC GTT TGG TGG ACC-BHQ1-3′

Subgenomic mRNA Assay

The qRT-PCR assay for sgRNA utilized primers and a probe specifically designed to amplify and bind to a region of the E gene messenger RNA from SARS-CoV-2. This is not packaged into the virion. The signal was compared to a known standard curve of plasmid and calculated to give copies per mL for the qRT-PCR assay.

The control DNA was prepared to contain 10⁷ copies per 3 μl. Seven (7) 10-fold serial dilutions of control RNA were prepared by adding 5 μl of the control to 45 μl of water and repeating this for 7 dilutions. This gave a standard curve with a range of 1 to 10⁶ copies/reaction. For amplification, the plate was placed in an Applied Biosystems 7500 Sequence detector and amplified using the following program: 48° C. for 30 minutes, 95° C. for 10 minutes followed by 40 cycles of 95° C. for 15 seconds, and 1 minute at 55° C. The number of copies of RNA per mL was calculated by extrapolating from the standard curve and multiplying by the reciprocal of 0.2 mL extraction volume. This gave a practical range of 50 to 5×10⁷ RNA copies per mL for nasal washes; for tissues, the viral loads are given per gram.

Primers:
SG-F:
(SEQ ID NO: 86)
CGATCTTGTAGATCTGTTCCTCAAACGAAC
SG-R:
(SEQ ID NO: 87)
ATATTGCAGCAGTACGCACACACA
Probe:
(SEQ ID NO: 88)
FAM-ACACTAGCCATCCTTACTGCGCTTCG-BHQ

Infectious Viral Load (TCID₅₀) Assay

Vero E6 cells (ATCC cat. no. CRL-1586) were plated at 25,000 cells/well in DMEM+10% FBS+Gentamicin and the cultures were incubated at 37° C., 5.0% CO₂. Cells were 80-100% confluent the following day. Medium was aspirated and replaced with 180 μL of DMEM+2% FBS+gentamicin. Twenty (20) L of sample were added to top row in quadruplicate and mixed using a P200 pipettor 5 times. Using the pipettor, 20 μL was transferred to the next row, and repeated down the plate (columns A-H) representing 10-fold dilutions. Positive (virus stock of known infectious titer in the assay) and negative (medium only) control wells were included in each assay set-up. The plates were incubated at 37° C., 5.0% CO₂ for 4 days. The cell monolayers were visually inspected for cytopathic effect (CPE). Non-infected wells have a clear confluent cell layer while infected cells have cell rounding. The TCID₅₀ value was calculated using the Read-Muench formula.

Luminex Assay for Serum and BAL Cytokines Chemokines

Serum and BAL samples were analyzed using a Cytokine/Chemokine/Growth Factor 37-Plex NHP ProcartaPlex™ Panel (EPX370-40045-901). The following analytes were reported: GS-CSF, IFN-gamma, IL-12, IL-6, IL1b, IL-4, IL-13, IL-5, IL-10, IL-2, MCP-1, and MIP-1b. The procedure followed the manufacturer's instructions.

Antibody ELISA for SARS-CoV-2 Spike Protein

A standard indirect ELISA was performed to analyze serum samples for binding antibodies to the SARS-CoV-2 Spike protein. For this assay, Nunc MaxiSorp 96-well plates (Thermo Scientific, Cat #439454) were coated with 50 μL of SARS-CoV-2 Spike protein (Sino Biological, cat. no. 40589-V08B1) diluted to 2 μg/mL in 1×Carbonate-Bicarbonate Buffer (CBB, Sigma, Cat #C3041-50CAP). Plates were incubated statically overnight at 2-8° C. Unbound coating antigen in each well was removed by washing 5 times with 200 μL with PBS+0.05% Tween-20. Plates were blocked with 100 μl of PBS+1% BSA. Once blocking was completed, blocking buffer was removed by inversion and each sample was plated. Plates were incubated for 1 hour at room temperature statically, followed by washing 5 times with 200 μL PBS+0.05% Tween-20 to remove unbound sera. 50 μL of the secondary detection antibody (Goat anti-Monkey IgG (H+L) Secondary Antibody, HRP, Invitrogen, PA1-84631) was added at a dilution of 1:10,000 and plates were incubated for 60 minutes at RT. Unbound antibodies were subsequently removed by washing 5 times with 200 μL with PBS+0.05% Tween-20 and 1 time with 200 μL of PBS. To develop, 100 μL of 1-Step Ultra TMB substrate (SERA CARE, KPL Cat #5120-0075) was added to each well. The plates were read within 30 minutes at 450 nm with a Thermo Labsystems Multiskan spectrophotometer.

Additional or Alternative Antibody ELISA for SARS-CoV-2 S, S1 and S2 Subunits, S-RBD, and N

Sandwich ELISA was performed to quantify serum IgG levels. For these assays, Nunc-Immuno MaxiSorp 96-well plates were coated with 4 μg/mL of SARS-CoV-2 S-RBD, his-tag (Wild-Type, SPD-C52H3, Acro biosystems), SARS-CoV-2 S-RBD (N501Y), his-tag (UK Mutant, SPD-C52Hn, Acro biosystems), SARS-CoV-2 S-RBD (K417N, E484K, N501Y), his-tag (South Africa Mutant, SPD-C82E5, Acro biosystems), SARS-CoV-2 S-P2 (S2 subunit of Spike), his-tag (Trimer, U of Washington, 35962), SARS-CoV-2 S-RBD Soluble, his-tag (U of Washington, 35961), SARS-CoV-1 S1 subunit of Spike (40150-V08B1, Sino Biological), SARS-CoV-2 Nucleoprotein, and influenza HA protein, and left overnight at room temperature. Similarly, AffiniPure F(ab′)2 Fragment-specific goat anti-human IgG (Jackson Laboratory) was coated for standards. IgG ELISA plates were washed (BioTek 405 TS) in 1×DPBS-T (0.05% Tween-20) and blocked with 1% bovine serum albumin (BSA) (Millipore Sigma) for 1 hour at RT. After blocking, plates were washed and 100 μL of diluted sera/purified human IgG (MP Biomedicals) were added to the antigen-coated plate and incubated for 1 hour at room temperature. Following washing, a Goat Anti-Human (H+L)-HRP (Bio-Rad) was diluted to 1:20,000 in 1×DPBS-T and added 100 μL/well to the plate. Plates were incubated for 1 hour at room temperature, while SureBlue TMB Microwell Peroxidase Substrate (VWR, Radnor, Pa., USA) equilibrated to room temperature. After a final wash, 100 μL TMB substrate was added to wells and development was stopped with 100 μL of 1N hydrochloric acid after 10 minutes at room temperature. The ELISA plates were read at an absorbance of 450 nm on a SpectraMax i3×Plate Reader using Softmax Pro 7.0.

Plaque Reduction Neutralization Test (PRNT)

The PRNT assay was conducted on serum samples. For this assay, Vero E6 cells (ATCC, cat #CRL-1586) were plated in 24-well plates at 175,000 cells/well in DMEM+10% FBS+Gentamicin. The plates were incubated at 37° C., 5.0% CO₂ until cells reached 80-100% confluency the following day. On the assay day, the serum samples were heat inactivated at 56° C. for 30 minutes. The assay set-up was performed as follows: In a 96 deep well plate, 405 μL of diluent (DMEM+2% FBS+gentamicin) was added to column 1 and 300 μL of diluent was added to columns 3, 5, 7, 9 and 11. Forty-five (45) μL of heat inactivated serum sample was added in the first column (1:10 dilution). When all samples had been added, the contents of the wells were mixed, and 150 μl was transferred to column 3 for a 1:3 fold dilution. This was repeated for the next set of wells down the plate to column 11. For the virus positive control, 300 L of diluent was added to columns 1, 3, 5, 7, 9 and 11 while 600 μL of diluent was added to row 1, representing the negative control.

A 30 pfu/well concentration of virus was prepared and kept on ice until use. After the titration plate had been prepared as described above, 300 μL of 30 pfu/well virus dilution was added to all samples and positive control wells. The plate was then covered with a plate sealer and incubated at 37° C., 5.0% CO₂ for 1 hour. After incubation, the media from the 24-well plate was removed and 250 μL of titrated samples was added in duplicate from the titration plate using a multichannel pipette. Only one plate was prepared at a time to avoid drying out the cells. The 24-well plates were incubated at 37° C., 5.0% CO₂ for 1 hour for virus infection. During this time, the 0.5% methylcellulose media was heated in a 37° C. water bath. After one hour of incubation, 1 mL of the 0.5% methylcellulose media was added to each well and the plates were incubated at 37°, 5% CO₂ for 3 days. The methylcellulose medium was removed, and the plates washed once with 1 mL PBS. The plates were fixed with 400 μL ice cold methanol per well at −20° C. for 30 minutes. After fixation, the methanol was discarded, and the monolayers stained with 250 μL per well of 0.2% crystal violet (20% MeOH, 80% dH2O) for 30 minutes at room temp. The plates were finally washed once with PBS or dH2O and let dry for ˜15 minutes. The plaques in each well were recorded and the IC50 and IC90 titers were calculated based on the average number of plaques detected in the virus control wells.

Additional or Alternative Plaque Reduction Neutralization Test (PRNT)

For these assays, a pre-titrated dose of virus was incubated with 8 serial 5-fold dilutions of serum samples in duplicate in a total volume of 150 μL for 1 hour at 37° C. in 96-well flat-bottom poly-L-lysine-coated Biocoat plates (Corning). Cells were suspended using TrypLE Select Enzyme solution (Thermo Fisher Scientific) and immediately added to all wells (10,000 cells in 100 μL of growth medium per well). One set of 8 control wells received cells+virus (virus control) and another set of 8 wells received cells only (background control) in a volume of 100 μL. After 66-72 hours of incubation, medium was removed by gentle aspiration and 30 μL of Promega 1×lysis buffer was added to all wells. After a 10-minute incubation at RT, 100 μL of Bright-Glo luciferase reagent was added to all wells. After 1-2 minutes, 110 μL of each cell lysate was transferred to a black/white plate (Perkin-Elmer). Luminescence was measured using a PerkinElmer Life Sciences, Model Victor2 luminometer. Neutralization titers are the serum dilution at which relative luminescence units (RLU) were reduced by 50% (ID₅₀) compared to virus control wells after subtraction of background RLUs. Serum samples were heat-inactivated for 30 minutes at 56° C. prior to the assay.

Antibody-Dependent Complement Deposition (ADCD)

Briefly, biotinylated antigen was coupled to fluorescent NeutrAvidin beads (Thermo Fisher Scientific, Waltham, Mass., USA). Plasma antibodies were diluted 1:10 in 0.1% BSA and incubated with the coupled antigen beads for 2 hours at 37° C. Beads were washed and incubated with complement factors from guinea pig for 20 minutes at 37° C. The complement reaction was then stopped by washing with 15 mM EDTA in PBS. C3 deposition on the beads was detected with a 1:100 diluted FITC-conjugated anti-guinea pig C3 polyclonal antibody (MP Biomedicals), and relative C3 deposition was analyzed by flow cytometry.

Antibody-Dependent Neutrophil Phagocytosis (ADNP)

Briefly, primary human neutrophils were obtained from ACK lysed blood of healthy donors. Biotinylated antigens were incubated with NeutrAvidin beads and immune complexes (ICs) formed by incubation with 1:100 diluted plasma for 2 hours at 37° C. in 96-well plates (Greiner Bio-One). Isolated neutrophils were added afterwards and incubated for 1 hour at 37° C. Neutrophils were surface stained against CD66b (1:50, Biolegend, clone: G10F5), fixed with 4% paraformaldehyde, and analyzed on an LSRII (BD) flow cytometer. Phagocytosis score was calculated as the product of frequency beads positive CD66b neutrophils and bead fluorescent intensity using FlowJo 10.8.

Antibody-Dependent Cellular Phagocytosis (ADCP)

THP-1 monocyte phagocytosis was performed as follows: Biotinylated antigens were conjugated to NeutrAvidin beads and incubated with 1:100 diluted plasma samples. THP-1 monocytes (0.25 million cells per well) were added to the immune complexes (ICs) and incubated for 16 hours at 37° C., fixed with 4% paraformaldehyde and analyzed by flow cytometry.

IgG Subclass, Isotype and FcR-Binding Luminex Profiling

IgG subclass and FcR profiling was conducted as follows: Antigens were carboxyl coupled to magnetic Luminex microplex carboxylated beads (Luminex Corporation) using NHS-ester linkages with Sulfo-NHS and EDC (Thermo Fisher), and then incubated with serum for 2 hours at room temperature. Subclass (IgG1 or IgG3) titer were first probed with a mouse rhesus-subclass IgG1 or IgG3 specific secondary antibody (NHP Reagent Resource), respectively; mouse IgG was then detected with a PE-conjugated anti-mouse antibody (Thermo-Fisher). FcR binding was quantified by incubating immune complexes with biotinylated FcRs (FcγR2A-1, FcγR2A-2, FcγR3A, Duke Protein Production Facility) conjugated to Steptavidin-PE (Prozyme/Agilent). Flow cytometry was performed with an IQue (Intellicyt), and analysis was performed on IntelliCyt ForeCyt (v8.1).

Results

Results are shown for MAPS Vaccine #1, comprising the RBD domain of the Spike glycoprotein from SARS CoV-2 (ancestral Wuhan D614G strain) fused with rhizavidin, and complexed with the well-characterized antigenic polysaccharide 1 (PS1) of Streptococcus pneumoniae.

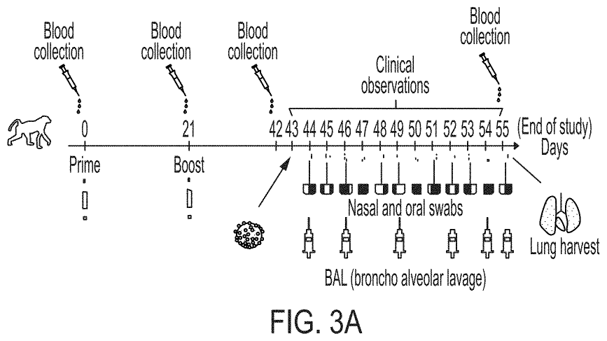

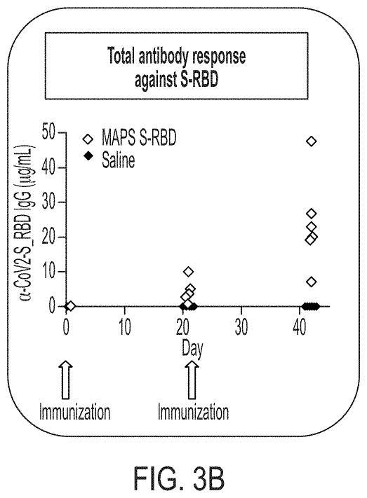

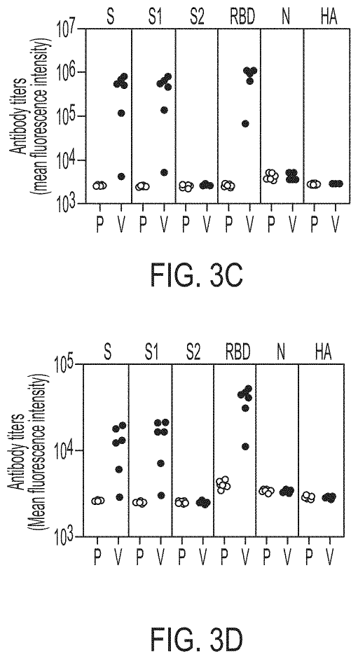

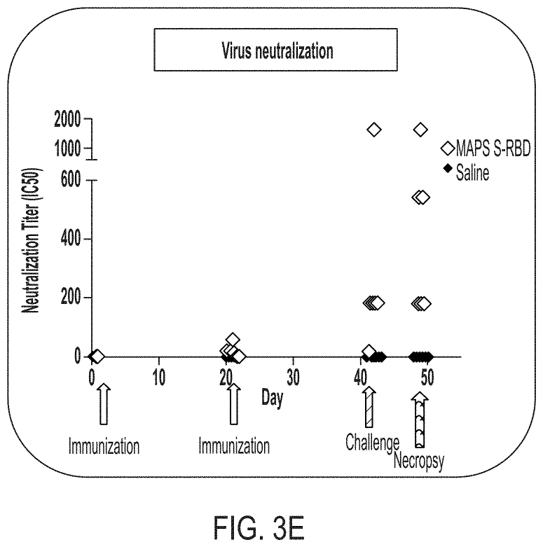

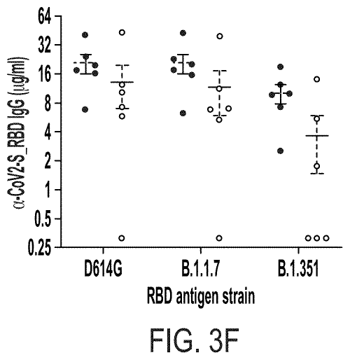

FIGS. 3A-3F shows robust antibody (B cell) responses and viral neutralization following immunization with MAPS Vaccine #1. FIG. 3A is a schematic of the study. FIGS. 3B, 3C, and 3D show total and specific IgG response against SARS-CoV-2 S-RBD and other targets, analyzed by ELISA at baseline (Day 0 of the study), 21 days post-first injection (Day 21, before the second injection; P1), and again 21 days post-second injection (Day 42; P2). Results are expressed as anti-S-RBD IgG titers in μg/mL serum. Each point on the graphs represents results for one animal. FIG. 3B: Total IgG levels against SARS-CoV-2 S-RBD in Day 0, Day 21, and Day 42 sera of saline- and vaccine-immunized animals. An increase in antibodies to S-RBD was observed after the first and second immunizations. FIGS. 3C and 3D: Subclass IgG1 levels (FIG. 3C) and subclass IgG3 levels (FIG. 3D) against SARS-CoV-2 Spike protein (S), S1 subunit, S2 subunit, S-RBD, Nucleoprotein (N), and unrelated influenza HA protein in Day 42 sera of saline- and vaccine-immunized animals. P=saline (open circles); V=vaccine (filled circles). IgG1 and IgG3 antibodies directed against S and S1 subunit were detected, but not against the S2 subunit, which is not included in the vaccine. The specificity of the measured response was further confirmed by the absence of ELISA signal to SARS-CoV-2 nucleoprotein (N) and the unrelated influenza HA protein. The results of FIGS. 3C and 3D provide additional confirmation of the results of FIG. 3B. FIG. 3E shows SARS-CoV-2 (ancestral Wuhan D614G strain) neutralization by the plaque reduction neutralization assay (PRNT) at Day 0, Day 21, Day 42, and Day 49 (upon necropsy), following immunization with saline or vaccine. Results are expressed as IC50 titer. In each case, data points for individual animals are plotted. An increase in neutralizing antibody responses was observed after the first and second immunizations. FIG. 3F shows cross-reactive antibody responses against S-RBD of different SARS-CoV-2 strains. The graph depicts IgG levels against S-RBD of strains D614G (ancestral Wuhan), B.1.1.7 (UK) or B.1.351 (South Africa) in Day 42 sera of vaccine-immunized animals (black circles) and sera collected from seroconverted human patients, recovered from COVID-19 (gray circles). The concentration of total IgG was highest against S-RBD of the D614G (ancestral Wuhan) strain compared to the two other tested strains. Similar results from obtained from sera of vaccine-immunized animals and from sera of seroconverted human patients.

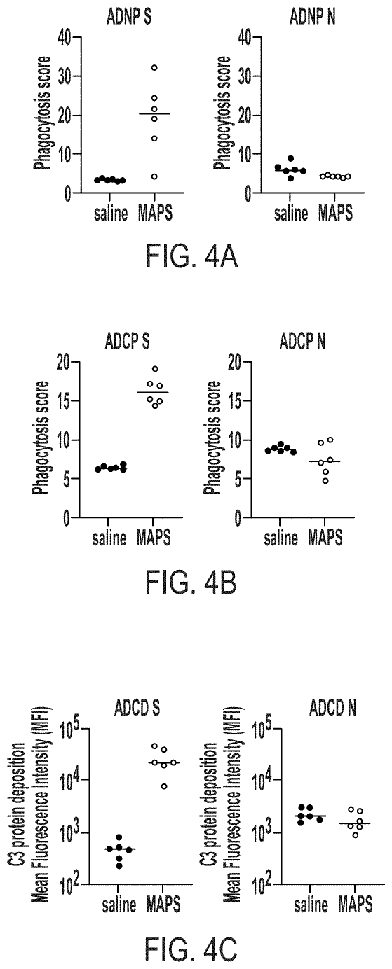

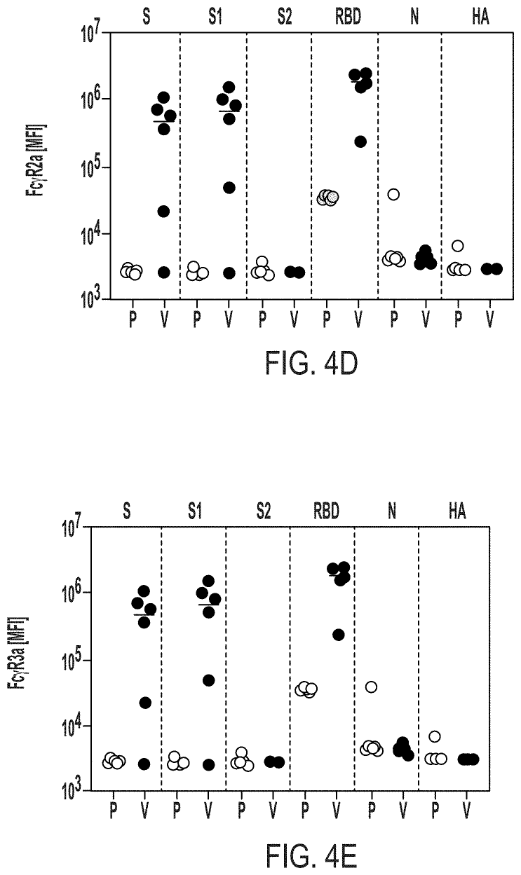

FIGS. 4A-4E show that antibodies generated against SARS-CoV-2 S-RBD following immunization with MAPS Vaccine #1 exhibit high Fc receptor binding and antibody effector functions. Opsonophagocytic and cytotoxic function depend on the ability of antibodies to interact with Fc-receptors found on immune cells. In humans and nonhuman primates, four low-affinity Fc receptors (FcγR2a, FcγR2b, FcγR3a, and FcγR3b) drive IgG-mediated activation. Functional anti-SARS-CoV-2 S-RBD antibody responses were evaluated by antibody-depended neutrophil phagocytosis (ADNP, FIG. 4A); antibody-dependent cellular phagocytosis (ADCP, FIG. 4B); and antibody-dependent complement deposition (ADCD, FIG. 4C), using the SARS-CoV-2 Spike protein (S) or Nucleoprotein (N) in Day 42 sera of saline- or vaccine-immunized animals. Assays were conducted using Luminex assays. Baseline antibody response was measured against SARS-CoV-2 N-RBD. FIGS. 4D and 4E show binding of SARS-CoV-2 specific-antibodies to Fcγ receptor 2A (FcγR2A) (FIG. 4D) and Fcγ receptor 3A (FcγR3A) (FIG. 4E) in the presence of SARS-CoV-2 Spike protein (S), S1 subunit, S2 subunit, S-RBD, Nucleoprotein (N), and unrelated influenza HA protein, in Day 42 sera of saline- and vaccine-immunized animals. P=saline (open circles); V=vaccine (filled circles). When compared to sera from NHPs that had received saline alone, sera from NHPs immunized with MAPS Vaccine #1 bound more to FcγR2a (FIG. 4D) and FcγR2b (FIG. 4E) in the presence of S, S1 or S-RBD proteins. No increase in binding was observed in the presence of control proteins S2, SARS-CoV-2 N-RBD, or unrelated influenza HA.

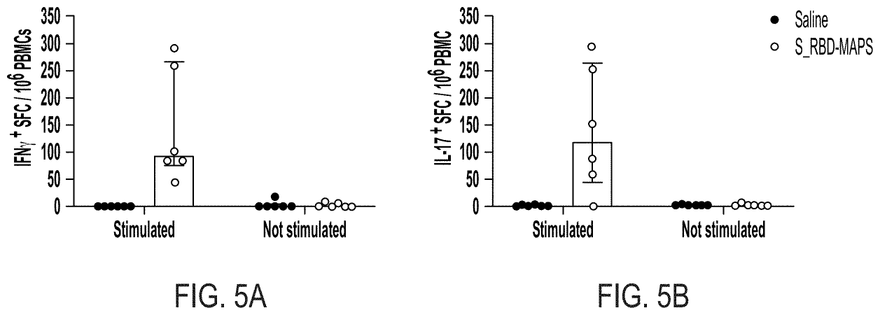

FIGS. 5A and 5B show the presence of IL-17 and IFN-7 secreting cells following immunization with MAPS Vaccine #1. PBMCs were collected and incubated with SARS-CoV-2 S-RBD protein (stimulated) or medium (not stimulated) for 42 hours. Frequency of IL-17 or IFN-γ secreting cells was measured by ELISpot. FIG. 5A shows results expressed as IFN-γ positive SFU/10⁶ PBMCs. FIG. 5B shows results expressed as IL-17 positive SFU/10⁶ PBMCs.

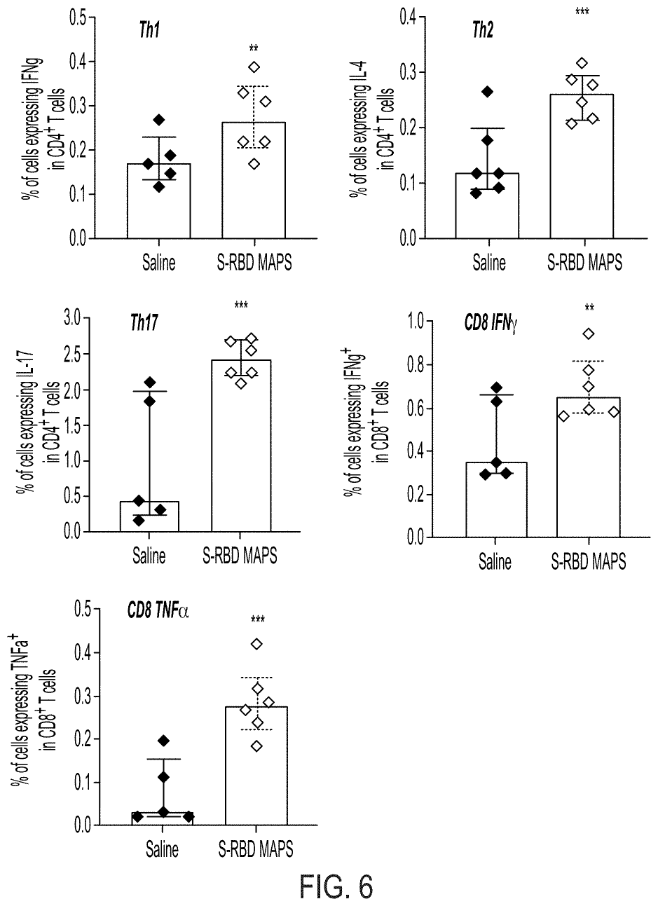

FIG. 6 shows induction of CD4+ and CD8+ T cell responses following two doses of MAPS Vaccine #1. PBMCs were collected and incubated with SARS-CoV-2 S-RBD protein (stimulated) or medium (not stimulated) for 42 hours. Frequency of Th1, Th2, Th17, or CD8+ T cells expressing IFN-γ or TNF-α was evaluated by flow cytometry. Results are expressed as the percentage of CD4+ T cells expressing IFN-7 (for Th1), IL-4 (for Th2), or IL-17 (for Th17), and of CD8+ T cells expressing IFN-7 or TNF-α. Individual data points as well as the median+/−IQR are plotted. Significance was tested using one-way ANOVA test. ns=not significant. *p<0.01, **p<0.005, ***p<0.0001.

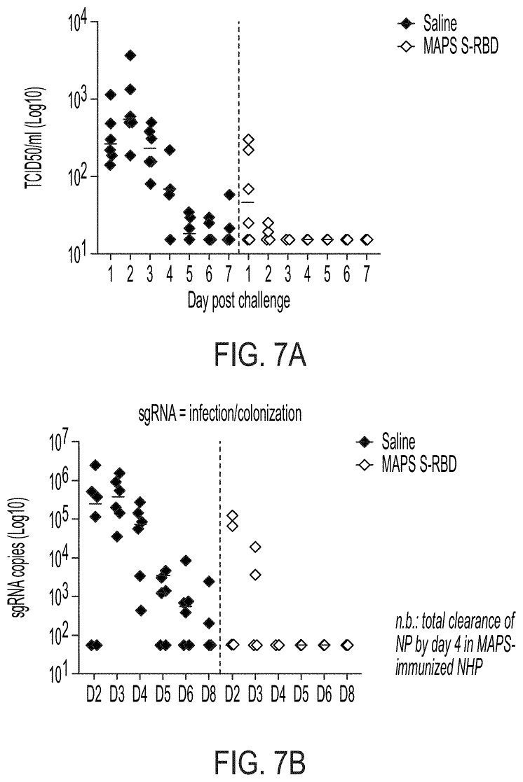

FIGS. 7A and 7B show efficacy of MAPS Vaccine #1 against nasopharyngeal viral replication and active viral shedding. Nasal swabs were collected on the indicated days after challenge. FIG. 7A shows viral replication assessed by the Tissue Culture Infectious Dose (TCID₅₀) assay on nasal swabs collected on each of days 1-7 after challenge. Individual data points as well as the median are plotted. FIG. 7B shows viral replication assessed by qRT-PCR analysis of SARS-CoV-2 subgenomic RNA (sgRNA) on nasal swabs collected on days 2, 3, 4, 6 and 8 after challenge. Results are expressed as sgRNA copy number on a log scale. In NHPs that received saline alone, viral RNA was detected in nasal swabs from Day 1 to Day 7 or 8 after challenge, by both TCID₅₀ and qRT-PCR. In NHPs that received vaccine, viral RNA was detected in 4/6 nasal swabs by TCID₅₀ on Day 1, but none by Day 3. By qRT-PCR, viral RNA was detected in 2/6 nasal swabs; by Day 4, all nasal swabs were negative.

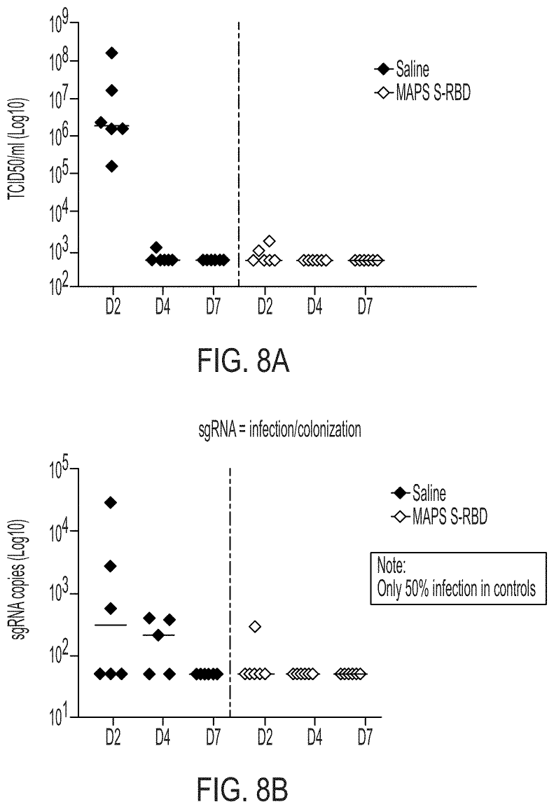

FIGS. 8A and 8B show efficacy of MAPS Vaccine #1 against pulmonary infection. Bronchio-pulmonary lavages (BAL) were performed on days 2, 4, and 7 after challenge. FIG. 8A shows viral replication assessed by the TCID₅₀ assay on BAL collected on the indicated days. Individual data points as well as the median are plotted. FIG. 8B shows viral replication assessed by analysis of SARS-CoV-2 subgenomic RNA (sgRNA) on BAL collected on the indicated days. Results are expressed as sgRNA copy number on a log scale. In NHPs that received saline alone, viral RNA was detected on Days 2 and 4 after challenge, by both TCID₅₀ and qRT-PCR. In NHPs that received vaccine, viral RNA was detected in only 1/6 BAL samples by qRT-PCR at Day 2. No viral RNA was detected at later timepoints.

These results demonstrate that in NHPs, a vaccine based on the MAPS vaccine platform and comprising the RBD domain of the Spike glycoprotein from SARS CoV-2 (ancestral Wuhan D614G strain) generated antibodies with robust Fc-effector functions (Fc receptor binding, phagocytosis, complement deposition); generated T cell responses, including Th1, Th2, Th17 and CTL (CD8+ IFN-7 and CD8+ TNF-α) responses; and reduced active viral replication and shedding in the nasopharyngeal passages (as determined by nasal swabs), as well as pulmonary infection (as determined by BAL).

A potential correlate of protection for neutralizing antibody levels has been established in NHPs and humans (McMahan et al., 2021. Correlates of protection against SARS-CoV-2 in rhesus macaques. Nature 590, 630-634; and Sui et al., 2021. Potential SARS-CoV-2 Immune Correlates of Protection in Infection and Vaccine Immunization. Pathogens 10, 138). The results shown here in cynomolgus macaques suggest that a MAPS vaccine comprising the RBD of Spike glycoprotein confers high levels of neutralizing antibodies and is protective in a stringent model of COVID-19.

### Example 2. Immunogenicity of a SARS-CoV-2 MAPS Vaccine

This study evaluated an exemplary SARS-CoV-2 MAPS vaccine in an animal model. In particular, this study evaluated SARS-CoV-2 MAPS Vaccine #1 in rabbits. MAPS Vaccine #1 comprises the RBD of the S glycoprotein from SARS CoV-2 (e.g., ancestral Wuhan D614G strain) fused with rhizavidin (e.g., in some embodiments, a fusion protein comprising SEQ ID NO:5), shown graphically in FIG. 2, top diagram. New Zealand rabbits (n=3) were immunized with two subcutaneous inoculations of MAPS Vaccine #1 at Day 0 and 21 (FIG. 2A). The antibody responses against SARS-CoV-2 S glycoprotein were measured on Day 0 (baseline before injection, P0), Day 21 (representing the response after first immunization, P1), and again on Day 42 (representing the response after second immunization, P2) by ELISA and plaque reduction neutralization assay (PRNT).

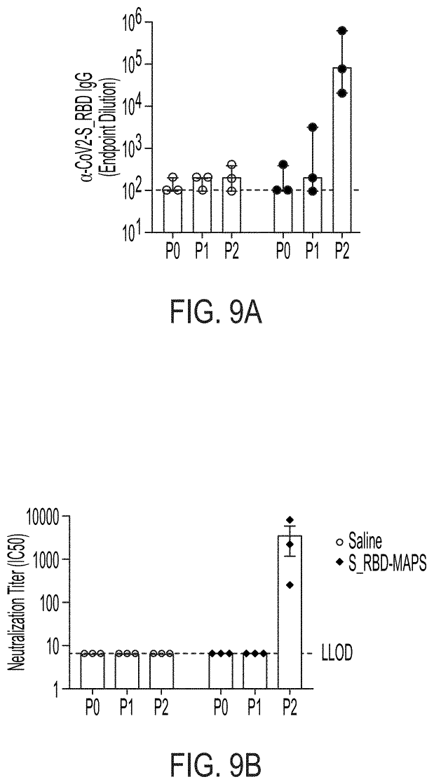

FIGS. 9A and 9B show total IgG levels in μg/mL (FIG. 9A) and IC50 neutralizing antibody titers (FIG. 9B) against SARS-CoV-2 S glycoprotein in P0 (Day 0), P1 (Day 2), and P2 (Day 42) sera of saline- or vaccine-immunized rabbits. Open circles=saline; black diamonds=vaccine. Serum concentration of antibodies to S and neutralizing antibody activity greatly increased after the second immunization with vaccine.

### Example 3: Evaluating Immunogenicity of Multivariant SARS-CoV-2 MAPS Vaccine Candidates

Study Objective

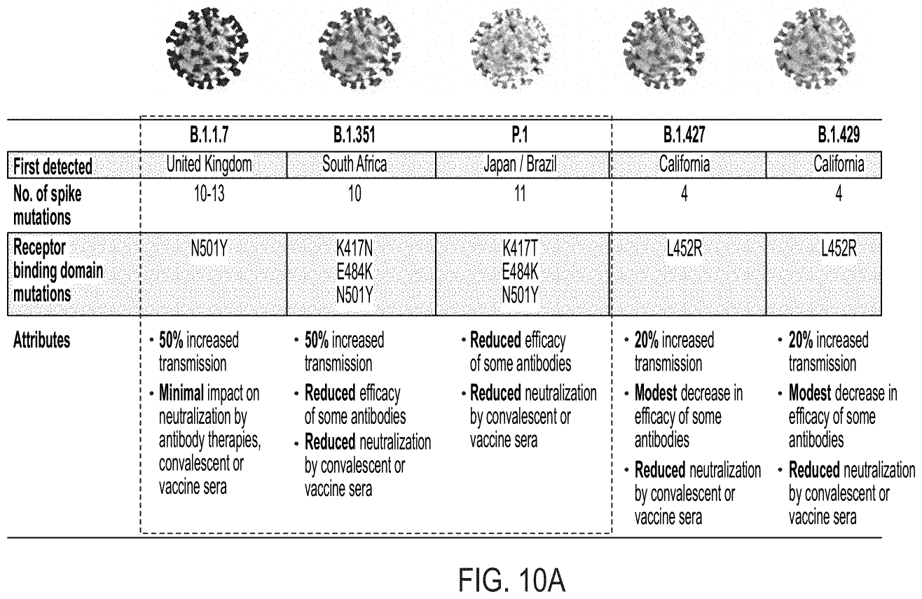

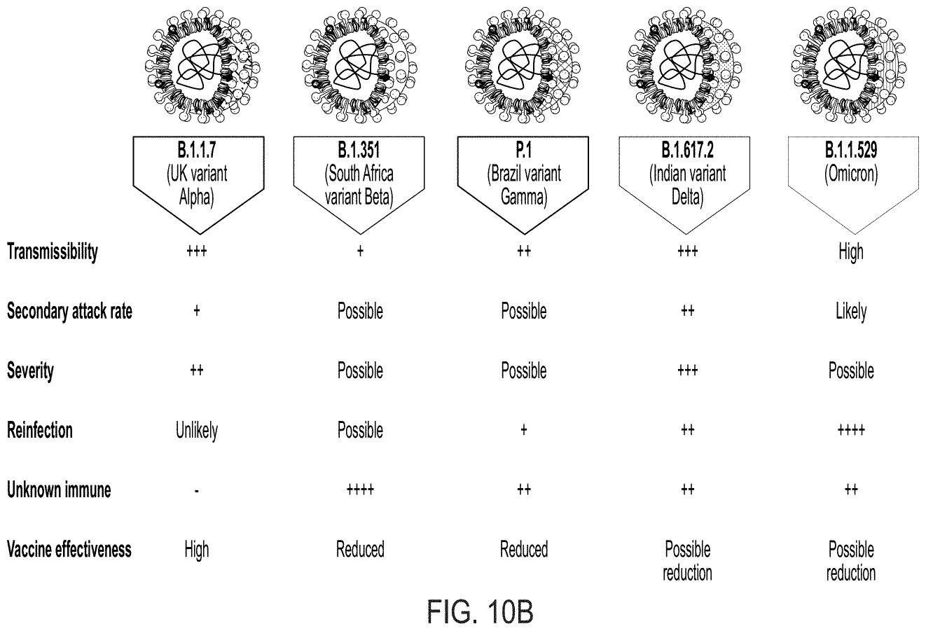

Following the positive proof of concept (POC) achieved with two candidate MAPS vaccines against SARS-CoV-2 (ancestral Wuhan D614G strain) in the cynomolgus macaque challenge model (Example 1), multivariant MAPS vaccine candidates were designed to target emerged variants of concern (VOCs). These include but are not limited to variants B.1.1.7 (UK, Alpha variant), B.1.351 (South Africa, Beta variant), P.1 (Japan/Brazil, Gamma variant), B.1.427/9 (California, Epsilon variant) and B.1.617.2 (India, Delta variant). Representative VOCs are presented in FIGS. 10A and 10B. In addition, multivariant MAPS vaccine candidates were designed to cover potential future variants of concern (VOCs) of SARS-CoV-2, for example, encompassing combinations of the following mutations of S glycoprotein RBD: K417N, L452R, E484K or E484Q, and N501Y.

Studies 1 and 2 evaluated immunogenicity of Multivariant MAPS Vaccine #1 targeting the ancestral Wuhan D614G, B.1.1.7 (UK, Alpha variant), and B.1.351 (South Africa, Beta variant) strains in rabbits and mice. Study 3 evaluated immunogenicity of Multivariant MAPS Vaccine #2 targeting the ancestral Wuhan D614G, B.1.617.2 (India, Delta variant), and P.1 (Japan/Brazil, Gamma variant) strains in mice. Study 4 evaluated Multivariant MAPS Vaccine #1 in a hamster challenge model. Additional studies evaluated immunogenicity of multivariant MAPS vaccines targeting additional emerged variants, as well as monovariant and multivariant MAPS vaccines targeting potential future VOCs.

Control and Test Articles

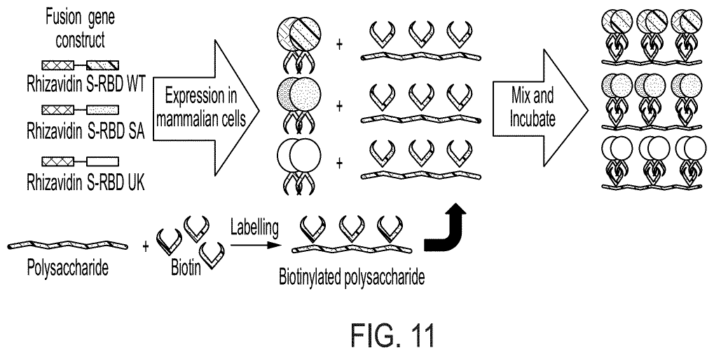

Studies 1 and 2 were designed to test candidate Multivariant MAPS Vaccine #1 against the SARS CoV-2 ancestral Wuhan D614G, B.1.1.7 (UK, Alpha variant), and B.1.351 (South African, Beta variant) strains. The multivariant vaccine comprised a mixture of three monovariant MAPS vaccines, as shown graphically in FIG. 11. Immunogenic complexes included in each monovariant MAPS vaccine comprised the RBD of the S glycoprotein from either the ancestral Wuhan D614G (SEQ ID NO:3), B.1.1.7 (UK, Alpha variant, SEQ ID NO:6), or B.1.351 (South Africa, Beta variant, SEQ ID NO:9) strains, fused with a rhizavidin polypeptide (e.g., in some embodiments a truncated rhizavidin protein that retains a biotin-binding domain), denoted Rhavi, as shown in FIG. 2 (top diagram).

In some embodiments, a multivariant MAPS vaccine can be developed to target against the ancestral Wuhan D614G, B.1.617.2 (India, Delta variant), and P.1 (Japan/Brazil, Gamma variant) strains. Similar to Multivariant MAPS Vaccine #1 as depicted in FIG. 11, such a multivariant MAPS vaccine comprises a mixture of three monovariant MAPS vaccines. In some embodiments, immunogenic complexes included in each monovariant MAPS vaccine comprise the RBD of the S glycoprotein from either the ancestral Wuhan D614G (SEQ ID NO:3), B.1.617.2 (India, Delta variant; SEQ ID NO:12), or P.1 (Japan/Brazil, Gamma variant; SEQ ID NO:18) strains, fused with a rhizavidin polypeptide, as shown in FIG. 2 (top diagram). In some embodiments, such a rhizavidin polypeptide can be a truncated rhizavidin protein that retains a biotin-binding domain. In some embodiments, such a rhizavidin polypeptide can be a rhizavidin variant.

Study 3 was designed to test candidate Multivariant MAPS Vaccine #2, targeting the ancestral Wuhan D614G, B.1.617.2 (India, Delta variant), and P.1 (Japan/Brazil, Gamma variant) strains in mice. Similar to Multivariant MAPS Vaccine #1 as depicted in FIG. 11, Multivariant MAPS Vaccine #2 comprised a mixture of three monovariant MAPS vaccines. Immunogenic complexes included in each monovariant MAPS vaccine comprised the RBD of the S glycoprotein from either the ancestral Wuhan D614G (SEQ ID NO:3), B.1.617.2 (India, Delta variant; SEQ ID NO:12), or P.1 (Japan/Brazil, Gamma variant; SEQ ID NO:18) strains, fused with a Rhavi polypeptide, as shown in FIG. 2 (top diagram).

Rhizavidin may be fused to the N-terminus or the C-terminus of the S-RBD. In Studies 1 and 2 of this Example, immunogenic complexes of each monovariant MAPS vaccine comprised fusion proteins comprising the amino acid sequence of SEQ ID NO:5, SEQ ID NO:8, or SEQ ID NO:11. Immunogenic complexes of each monovariant MAPS vaccine in Studies 1-4 also comprised the well-characterized antigenic polysaccharide 1 (PS1) of Streptococcus pneumoniae. Placebo groups received adjuvant (aluminum hydroxide, AlOH) or PBS only.

Study Design and Results

Study 1: White New Zealand Rabbits

Test article: Multivariant MAPS Vaccine #1: 50 μg of protein in MAPS immunogenic complexes per 1 mL dose (0.05 mg/mL), formulated in 1.2 mL of 20 mM tris, pH 8.0, 150 mM sodium chloride, 0.25 μg ALHYDROGEL (AlOH).

Each rabbit was pre-bled (˜20 mL) before immunization. Each rabbit received one subcutaneous injection of 1.0 mL of formulated test article or saline control on each day of immunization, for a total of 2 immunizations, 3 weeks apart. Each rabbit was bled after the 1^{st} immunization (prior to 2^{nd} immunization) and exsanguinated 3 weeks after the 2^{nd} immunization. Rabbit sera were analyzed by ELISA and by a surrogate neutralization assay authorized by the FDA for emergency use (Tan et al., 2020. A SARS-CoV-2 surrogate virus neutralization test based on antibody-mediated blockage of ACE2-Spike protein-protein interaction. Nat. Biotechnol. 38, 1073-1078). Each serum was assayed for total IgG level and neutralizing antibodies against the RBD of the S glycoprotein from each of the ancestral Wuhan D614G, B.1.1.7 (UK, Alpha variant), and B.1.351 (South Africa, Beta variant) strains. The surrogate neutralizing assay is based on inhibition of S-RBD binding to the hACE2 receptor, measured in the presence of serially diluted serum. Neutralizing antibody titer for each sample is expressed as IC50, the titer resulting in 50% S-RBD-hACE2 receptor binding inhibition.

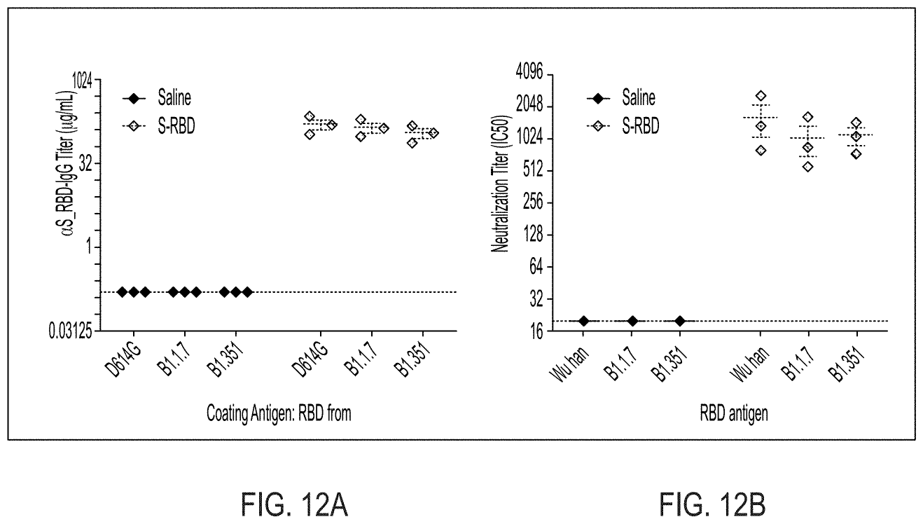

FIGS. 12A and 12B show total IgG levels in μg/mL (FIG. 12A) and IC50 neutralizing antibody titers (FIG. 12B) against S-RBD of the indicated strain. Individual data points as well as the mean+/−SD are shown. Immunization with Multivariant MAPS Vaccine #1 elicited high concentrations of total IgG and neutralizing antibody titers against each ancestral and variant S-RBD in rabbits.

Study 2: C57Bl6/J Mice

Test articles: Monovariant MAPS vaccines against S-RBD of ancestral Wuhan D614G, B.1.1.7 (UK, Alpha variant), and B.1.351 (South Africa, Beta variant) strains, and Multivariant MAPS Vaccine #1: 20 μg of protein in MAPS immunogenic complexes per 0.1 mL dose (0.2 mg/mL), formulated in 0.1 mL of 20 mM tris, pH 8.0, 150 mM sodium chloride, 0.25 μg ALHYDROGEL (AlOH).

Each mouse received one subcutaneous injection of 100 μL of formulated vaccine or PBS control on each day of immunization, for a total of 2 immunizations, 3 weeks apart. Each mouse was bled every 7 days after the 1^{st} immunization, i.e. on Days 7, 14, 21, and 28 of the study. Mouse sera were analyzed by ELISA and by surrogate neutralization assay authorized by the FDA for emergency use (Tan et al., 2020. A SARS-CoV-2 surrogate virus neutralization test based on antibody-mediated blockage of ACE2-Spike protein-protein interaction. Nat. Biotechnol. 38, 1073-1078). Each serum was assayed for total IgG level and neutralizing antibodies against RBD of the S glycoprotein from each of the ancestral Wuhan D614G, B.1.1.7 (UK, Alpha variant), and B.1.351 (South Africa, Beta variant) strains. The surrogate neutralizing assay is based on inhibition of S-RBD binding to the hACE2 receptor, measured in the presence of serially diluted serum. Neutralizing antibody titer for each sample is expressed as IC50, the titer resulting in 50% S-RBD-hACE2 receptor binding inhibition.

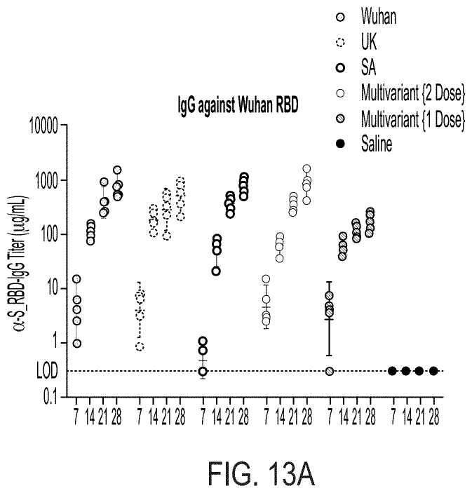

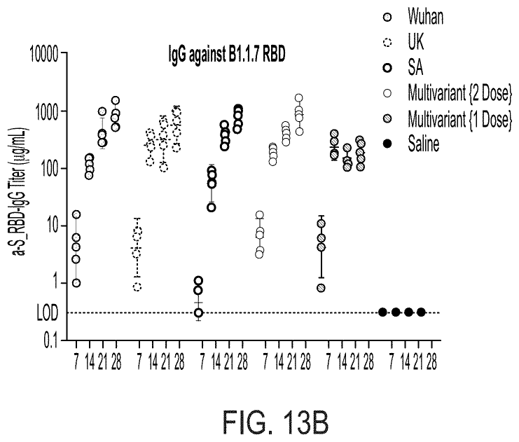

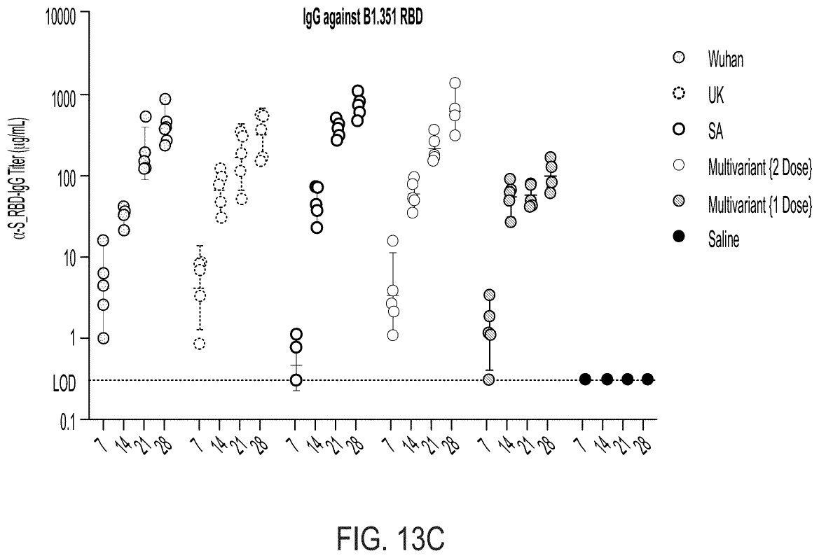

FIGS. 13A-13C show total IgG levels in μg/mL against S-RBD of the ancestral Wuhan D614G, B.1.1.7 (UK, Alpha variant), and B.1.351 (South Africa, Beta variant) strains, following immunization with the indicated monovariant MAPS vaccines or Multivariant MAPS Vaccine #1. Individual data points as well as the mean+/−SD are shown. Immunization with the monovariant MAPS vaccines and Multivariant MAPS Vaccine #1 elicited high concentrations of total IgG against each ancestral and variant S-RBD in mice.

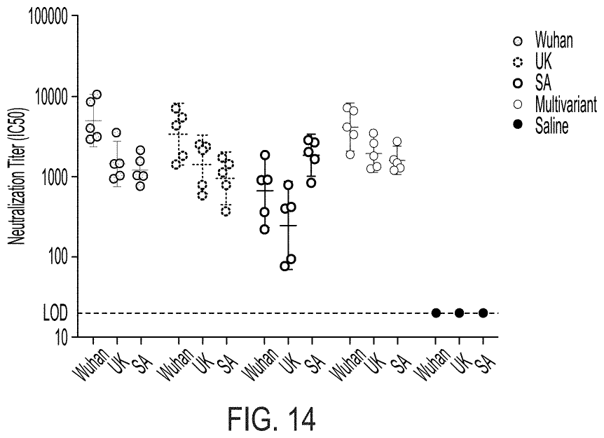

FIG. 14 shows IC50 neutralizing antibody titer against RBD of the ancestral Wuhan D614G, B.1.1.7 (UK, Alpha variant), and B.1.351 (South Africa, Beta variant) strains, following immunization with the indicated monovariant MAPS vaccines or Multivariant MAPS Vaccine #1. Individual data points as well as the mean+/−SD are shown. Immunization with the monovariant MAPS vaccines and Multivariant MAPS Vaccine #1 elicited high titers of neutralizing antibodies against each ancestral and variant S-RBD in mice.

As discussed in Example 1, a potential correlate of protection for neutralizing antibody levels has been established in NHPs and humans (McMahan et al., 2021. Correlates of protection against SARS-CoV-2 in rhesus macaques. Nature 590, 630-634; and Sui et al., 2021. Potential SARS-CoV-2 Immune Correlates of Protection in Infection and Vaccine Immunization. Pathogens 10, 138). The levels of neutralizing antibodies shown here in rabbits and mice suggest that monovariant MAPS vaccines and Multivariant MAPS Vaccine #1 may be protective against the three targeted variants. These results also demonstrate the potential to mix-and-match monovariant MAPS vaccines, perhaps with different degrees of cross-reactivity, in order to achieve broad protection against emerging VOCs. Confirmatory studies in a hamster challenge model are described below.

Study 3: C57Bl6/J Mice

Test articles: Monovariant MAPS vaccines against S-RBD of ancestral Wuhan D614G, B.1.617.2 (India, Delta variant), and P.1 (Japan/Brazil, Gamma variant) strains, and Multivariant MAPS Vaccine #2, using a Rhavi polypeptide: 20 μg of protein in MAPS immunogenic complexes per 0.2 mL dose, formulated in 0.1 mL of 20 mM Tris, pH 8.0, 150 mM sodium chloride, 0.25 μg ALHYDROGEL (AlOH).

Each mouse received one subcutaneous injection of 200 μL of formulated vaccine or PBS control on each day of immunization, for a total of 2 immunizations, 3 weeks apart. Each mouse was bled on Days 21, 42, and 56 of the study. Mouse sera were analyzed by ELISA. Each serum was assayed for total IgG level against RBD of the S glycoprotein from each of the ancestral Wuhan D614G, B.1.617.2 (India, Delta variant), and P.1 (Japan/Brazil, Gamma variant) strains.

Immunization with the monovariant MAPS vaccines and Multivariant MAPS Vaccine #2, containing one third of the dose of each monovariant MAPS vaccine, elicited similarly high concentrations of total IgG against ancestral and variant S-RBD in mice. These results demonstrate the feasibility of multivalent and/or multivariant MAPS vaccines against SARS-CoV-2.

Study 4: Hamster Challenge Model

Test article: Monovariant MAPS vaccines and Multivariant MAPS Vaccine #1: 20 μg of protein in MAPS immunogenic complexes per 0.1 mL dose (0.2 mg/mL), formulated in 0.1 mL of 20 mM tris, pH 8.0, 150 mM sodium chloride, 0.25 μg ALHYDROGEL (AlOH).

Each hamster received one intramuscular injection of 100 mL of formulated vaccine or PBS control on each day of immunization, for a total of 2 immunizations, 3 weeks apart. Each hamster was bled 21 days after the first immunization, and 14 days after the second immunization. Fourteen days after immunization with the second dose, the hamsters were challenged with the SARS-CoV-2 virus (Delta variant) via the combination intranasal/intratracheal (IN/IT) route, and clinical signs were measured from day 36 until day 43. Nasal swabs were collected and viral load in the nasopharynx was assessed by the Tissue Culture Infectious Dose (TCID) assay and by analysis of SARS-CoV-2 subgenomic RNA (sgRNA) at days 1, 3, 5, and 7 after challenge. In addition, at day 43, tissues (lung, heart, spleen, liver) were collected and viral load was assessed.

### Example 4: B Cell and T Cell Studies of Mice Vaccinated with SARS-CoV-2 MAPS Vaccine

The purpose of these studies was to determine whether immunization with exemplary MAPS vaccine as described herein against SARS-CoV-2 (ancestral Wuhan D614G) generates a memory B cell response, and if so, whether CD4+ T cells are implicated in the memory B cell response.

Test article: Monovariant MAPS vaccine comprising the RBD of the S glycoprotein from SARS CoV-2 (ancestral Wuhan D614G strain), fused with a Rhavi polypeptide, denoted MAPS Vaccine #3: 20 μg of protein in MAPS immunogenic complexes per 0.2 mL dose, formulated in 0.1 mL of 20 mM tris, pH 8.0, 150 mM sodium chloride, 0.25 μg ALHYDROGEL (AlOH). See FIG. 2 (top diagram) and Example 3.

Each C57Bl6/J mouse received one subcutaneous injection of 200 μL of formulated vaccine or PBS control on each day of immunization, for a total of 2 immunizations, 3 weeks apart. Each mouse was bled on Days 0, 21, 42, 60, and 90, and sacrificed on Day 180 of the study. Bone marrow and spleens were harvested. Mouse sera were assayed by ELISA for total IgG level against S-RBD of the ancestral Wuhan D614G strain.

Immunization with MAPS Vaccine #3 elicited a robust and durable IgG response against S-RBD of the ancestral Wuhan D614G strain. The sera collected from mice immunized with MAPS Vaccine #3 showed an anti-SARS-CoV-2 RBD IgG titer of at least 10 μg/mL (e.g., between 10 μg/mL and 500 μg/mL) by day 42 of the study which persisted until day 180 when the animals were sacrificed. Immunization with saline alone did not show an IgG response.

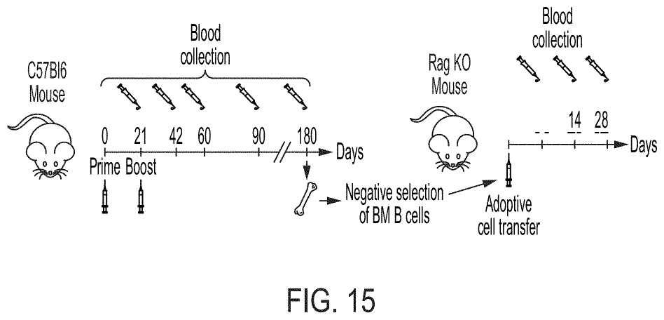

For Study 1, B cells were collected from bone marrow by negative selection using magnetic beads and washed 2× with PBS to eliminate potential residual antibodies. Purified B cells (10⁶) were resuspended in 100 μL of PBS and injected intravenously via the retroorbital vein into T cell- and B cell-deficient RAG2 KO mice (i.e., adoptively transferred). Sera collected on Days 7, 14, and 28 following adoptive transfer were assayed by ELISA for total IgG level against S-RBD of the ancestral Wuhan D614G strain. FIG. 15 shows the design of adoptive transfer Study 1.

RAG2 KO mice receiving bone marrow-derived B cells from MAPS Vaccine #3-vaccinated C57Bl6/J mice developed an IgG humoral response against S-RBD of the ancestral Wuhan D614G strain, with some mice showing an anti-SARS-CoV-2 RBD IgG titer of greater than 10 μg/mL in sera by day 7, and a majority or all of the mice showing a comparable or higher anti-SARS-CoV-2 RBD IgG titer by day 14 following adoptive transfer. In contrast, RAG2 KO mice receiving bone marrow-derived B cells from saline-vaccinated C57Bl6/J mice did not show an IgG humoral response. As RAG2 KO mice are unable to mount de novo immune responses due to the absence of B and T cells, these results demonstrate that immunization with a MAPS vaccine described herein (e.g., MAPS Vaccine #3) generated memory B cells against S-RBD. The transfer of such memory B cells led to a persistent IgG humoral response against S-RBD in the recipient mice.

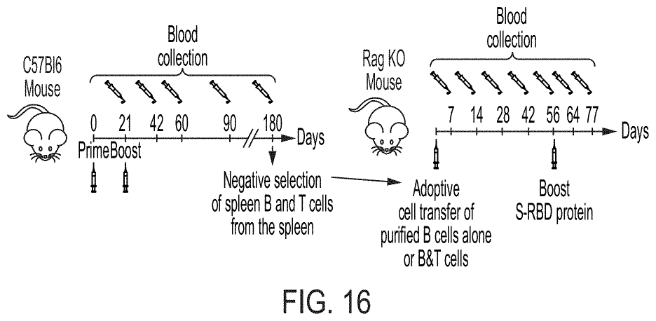

For Study 2, B cells and T cells were collected from spleens by negative selection using magnetic beads and washed 2× with PBS to eliminate potential residual antibodies. Purified B cells (10⁶) alone, or purified B cells (10⁶) and CD4+ T cells (10⁵) were resuspended in PBS and injected intravenously via the retroorbital vein into T cell- and B cell-deficient RAG2 KO mice (i.e., adoptively transferred). On Day 56 following adoptive transfer, all mice received a booster immunization of 10 μg S-RBD purified protein of the ancestral Wuhan D614G strain, adjuvanted with aluminum. Sera collected on Days 7, 14, 28, 42, 56, 64 and 77 following adoptive transfer were assayed by ELISA for total IgG level against S-RBD of the ancestral Wuhan D614G strain. FIG. 16 shows the design of adoptive transfer Study 2.

The results showed that approximately 50% of RAG2 KO mice that received spleen B cells and CD4+(helper) T cells from MAPS Vaccine #3-vaccinated C57Bl6/J mice developed an IgG humoral response against S-RBD of the ancestral Wuhan D614G strain at a IgG level of greater than 10 μg/mL, for example, as high as over 1000 μg/mL, by day 28 following adoptive transfer. Boosting the passively immunized mice with S-RBD protein increased both the overall rate of IgG response (from 50% to 100% of recipient mice) and the total IgG response (from mean of about 10 μg/ml to approximately 1000 μg/ml by Day 77). Mice that received B cells alone did not show an IgG humoral response.

All together, these results demonstrate that (i) a MAPS vaccine described herein (e.g., MAPS Vaccine #3) induced both B cells and CD4+(helper) T cells in the initially immunized mice; (ii) the presence of CD4+(helper) T cells was beneficial for IgG responses to S-RBD, at least in passively immunized mice; and (iii) IgG responses to S-RBD were greatly amplified following exposure of passively immunized mice to S-RBD protein, indicating that transferred B cells included memory B cells.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

LIST OF SEQUENCES

SEQ ID NO: 1, Rhizavidin protein, full-length [amino acids 1-179]
MIITSLYATFGTIADGRRTSGGKTMIRTNAVAALVFAVATSALAFDASNFKDFSSIASASSSWQ
NQSGSTMIIQVDSFGNVSGQYVNRAQGTGCQNSPYPLTGRVNGTFIAFSVGWNNSTENCNSATG
WTGYAQVNGNNTEIVTSWNLAYEGGSGPAIEQGQDTFQYVPTTENKSLLKD
SEQ ID NO: 2, truncated rhizavidin protein, denoted Rhavi [amino
acids 45-179]
FDASNFKDFSSIASASSSWQNQSGSTMIIQVDSFGNVSGQYVNRAQGTGCQNSPYPLTGRVNGT
FIAFSVGWNNSTENCNSATGWTGYAQVNGNNTEIVTSWNLAYEGGSGPAIEQGQDTFQYVPTTE
NKSLLKD
SEQ ID NO: 3, SARS-CoV-2 Spike glycoprotein RBD (Ancestral Wuhan
D614G)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TVCGP
SEQ ID NO: 4, Rhavi-S-RBD (Ancestral Wuhan D614G)
[Rhavi-linker-Spike glycoprotein RBD (Ancestral Wuhan D614G)]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQSYGFQPTNGVGYQPY
RVVLSFELLHAPATVCGP
SEQ ID NO: 5, S-RBD (Ancestral Wuhan D614G)-Rhavi
[Spike glycoprotein RBD (Ancestral Wuhan D614G)-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 6, SARS-CoV-2 Spike glycoprotein RBD (Alpha, B.1.1.7,
UK) (N501Y)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTYGVGYQPYRVVVLSFELLHAPA
TVCGP
SEQ ID NO: 7, Rhavi-S-RBD Alpha
[Rhavi-1inker-Spike glycoprotein RBD Alpha]
FDASNFKDFSSIASASSSWQNQSGSTMIIQVDSFGAVSGQYVNRAQGTGCQNSPYPLTGRVNGA
GAFIAFSVGWNAATENCNSATGWTGYAQVNGANTEIVTSWNLAYEGGSGPAIEQGQDTFQYVPT
TENKSLLKDGGGGSSSNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFPLQSYGFQPTYGVGYQ
PYRVVVLSFELLHAPATVCGP
SEQ ID NO: 8, S-RBD Alpha-Rhavi
[Spike glycoprotein RBD Alpha-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 9, SARS-CoV-2 Spike glycoprotein RBD (Beta, B.1.351,
South Africa) (K417N, E484K, N501Y)
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPA
TVCGP
SEQ ID NO: 10, Rhavi-S-RBD Beta
[Rhavi-1inker-Spike glycoprotein RBD Beta]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCVIAWNSNNLDSK
VGGNYNYLYRLFRKSNLKPFERDISTEIYQAGSTPCNGVKGFNCYFPLQSYGFQPTYGVGYQPY
RVVLSFELLHAPATVCGP
SEQ ID NO: 11, S-RBD Beta-Rhavi
[Spike glycoprotein RBD Beta-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 12, SARS-CoV-2 Spike glycoprotein RBD (Delta,
B.1.617.2, India) (L452R, T478K)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPA
TVCGP
SEQ ID NO: 13, Rhavi-S-RBD Delta
[Rhavi-linker-Spike glycoprotein RBD Delta]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNKKPFERDISTEIYQAGSKPCNGVEGFNCYFPLQSYGFQPTNGVGYQPY
RVVVLSFELLHAPATVCGP
SEQ ID NO: 14, S-RBD Delta-Rhavi
[Spike glycoprotein RBD Delta-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 15, SARS-CoV-2 Spike glycoprotein RBD (Delta plus,
AY.1, India) (K417N, L452R, T478K)
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPA
TVCGP
SEQ ID NO: 16, Rhavi-S-RBD Delta plus
[Rhavi-linker-Spike glycoprotein RBD Delta plus]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCIVAWNSNNLDSK
VGGNYNYRYRLFRKSNLKPFERDISTEIYQAGSKPCNGVEGFNCYFPLQSYGFQPTNGVGYQPY
RVWLSFELLHAPATVCGP
SEQ ID NO: 17, S-RBD Delta plus-Rhavi
[Spike glycoprotein RBD Delta plus-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSKPCNGVEGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 18, SARS-CoV-2 Spike glycoprotein RBD (Gamma, P.1,
Japan/Brazil) (K417T, E484K, N501Y)
NITNLCPFGEVFNATRFASVYAWNRKRISNCVADYSVLYNSASFSTFKCYGVSPTKLNDLCFTN
VYADSFVIRGDEVRQIAPGQTGTIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPA
TVCGP
SEQ ID NO: 19, Rhavi-S-RBD Gamma
[Rhavi-linker-Spike glycoprotein RBD Gamma]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGTIADYNYKLPDDFTGCVIAWNSNNLDSK
VGGNYNYLYRLFRKSNLKPFERDISTEIYQAGSTPCNGVKGFNCYFPLQSYGFQPTYGVGYQPY
RVVLSFELLHAPATVCGP
SEQ ID NO: 20, S-RBD Gamma-Rhavi
[Spike glycoprotein RBD Gamma-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGTIADYNYKLPDDFTGCVIAWNSNNLDSKVGGNYNYLYRLFRK
SNLKPFERDISTEIYQAGSTPCNGVKGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 21, SARS-CoV-2 Spike glycoprotein RBD (Epsilon,
B.1.427, B.1.429, California) (L452R)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVEGFNCYFPLQSYGFQPTNGVGYQPYRVVVLSFELLHAPA
TVCGP
SEQ ID NO: 22, Rhavi-S-RBD Epsilon
[Rhavi-linker-Spike glycoprotein RBD Epsilon]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNLKPFERDISTIEYQAGSTPCNGVEGFNCYFPLQSYGFQPTNGVGYQPY
RVWLSEELLHAPATVCGP
SEQ ID NO: 23, S-RBD Epsilon-Rhavi
[Spike glycoprotein RBD Epsilon-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVEGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 24, SARS-CoV-2 Spike glycoprotein RBD (Eta, B.1.525,
UK/Nigeria) (E484K)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVKGFNCYFPLQSYGFQPTNGVGYQPYRVVVLSFELLHAPA
TVCGP
SEQ ID NO: 25, Rhavi-S-RBD Eta
[Rhavi-linker-Spike glycoprotein RBD Eta]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVKGFNCYFPLQSYGFQPTNGVGYQPY
RVWLSFELLHAPATVCGP
SEQ ID NO: 26, S-RBD Eta-Rhavi
[Spike glycoprotein RBD Eta-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVKGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 27, SARS-CoV-2 Spike glycoprotein RBD (Iota, B.1.526,
US/NY) (L452R, E484K)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVKGFNCYFPLQSYGFQPTNGVGYQPYRVVVLSFELLHAPA
TVCGP
SEQ ID NO: 28, Rhavi-S-RBD Iota
[Rhavi-linker-Spike glycoprotein RBD Iota]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNLKPFERDISTEIYQAGSTPCNGVKGFNCYFPLQSYGFQPTNGVGYQPY
RVWLSFELLHAPATVCGP
SEQ ID NO: 29, S-RBD lota-Rhavi
[Spike glycoprotein RBD Iota-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKYGGNYNYRYRLFRK
SNLKPFERDISTEIYOAGSTPCNGVKGFNCYFPLOSYGFOPTNGVGYOPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 30, SARS-CoV-2 Spike glycoprotein RBD (Kappa,
B.1.617.1, India) (L452R, E484Q)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVQGFNCYFPLQSYGFQPTNGVGYQPYRVVVLSFELLHAPA
TVCGP
SEQ ID NO: 31, Rhavi-S-RBD Kappa
[Rhavi-linker-Spike glycoprotein RBD Kappa]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNLKPFERDISTEIYQAGSTPCNGVQGFNCYFPLQSYGFQPTNGVGYQPY
RVWLSFELLHAPATVCGP
SEQ ID NO: 32, S-RBD Kappa-Rhavi
[Spike glycoprotein RBD Kappa-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVQGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGODTFQYVPTTENKSLLKD
SEQ ID NO: 33, SARS-CoV-2 Spike glycoprotein RBD potential VOC #1
(K417N, L452R, E484Q, N501Y)
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSTPCNGVQGFNCYFPLQSYGFQPTYGVGYQPYRVVVLSFELLHAPA
TVCGP
SEQ ID NO: 34, Rhavi-S-RBD potential VOC #1
[Rhavi-linker-Spike glycoprotein RBD potential VOC #1]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCVIAWNSNNLDSK
VGGNYNYRYRLFRKSNLKPFERDISTEIYQAGSTPCNGVQGFNCYFPLQSYGFQPTYGVGYQPY
RVWLSFELLHAPATVCGP
SEQ ID NO: 35, S-RBD potential VOC #1-Rhavi
[Spike glycoprotein RBD potential VOC #1-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSTPCNGVQGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 36, SARS-CoV-2 Spike glycoprotein RBD potential VOC
#2 (K417N, L452R, E484K, N501Y)
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSTPCNGVKGFNCYFPLQSYGFQPTYGVGYQPYRVVVLSFELLHAPA
TVCGP
SEQ ID NO: 37, Rhavi-S-RBD potential VOC #2
[Rhavi-linker-Spike glycoprotein RBD potential VOC #2]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGNIADYNYKLPDDFTGCVIAWNSNNLDSK
VGGNYNYRYRLFRKSNLKPFERDISTEIYQAGSTPCNGVKGFNCYFPLQSYGFQPTYGVGYQPY
RVWLSFELLHAPATVCGP
SEQ ID NO: 38, S-RBD potential VOC #2-Rhavi
[Spike glycoprotein RBD potential VOC #2-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGNIADYNYKLPDDFTGCVIAWNSNNLDSKVGGNYNYRYRLFRK
SNLKPFERDISTEIYQAGSTPCNGVKGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 39, SARS-CoV-2 Spike glycoprotein RBD potential VOC #3
(L452R, E484Q, N501Y)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVQGFNCYFPLQSYGFQPTYGVGYQPYRVVVLSFELLHAPA
TVCGP
SEQ ID NO: 40, Rhavi-S-RBD potential VOC #3
[Rhavi-linker-Spike glycoprotein RBD potential VOC #3]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNLKPFERDISTEIYQAGSTPCNGVQGFNCYFPLQSYGFQPTYGVGYQPY
RVVLSFELLHAPATVCGP
SEQ ID NO: 41, S-RBD potential VOC #3-Rhavi
[Spike glycoprotein RBD potential VOC #3-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVQGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 42, SARS-CoV-2 Spike glycoprotein RBD potential VOC #4
(L452R, E484K, N501Y)
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVKGFNCYFPLQSYGFQPTYGVGYQPYRVVVLSFELLHAPA
TVCGP
SEQ ID NO: 43, Rhavi-S-RBD potential VOC #4
[Rhavi-linker-Spike glycoprotein RBD potential VOC #4]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYRYRLFRKSNLKPFERDISTEIYQAGSTPCNGVKGFNCYFPLQSYGFQPTYGVGYQPY
RVVLSFELLHAPATVCGP
SEQ ID NO: 44, S-RBD potential VOC #4-Rhavi
[Spike glycoprotein RBD potential VOC #4-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYRYRLFRK
SNLKPFERDISTEIYQAGSTPCNGVKGFNCYFPLQSYGFQPTYGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKD
SEQ ID NO: 45, SARS-CoV-2 M protein fused ECD1 and ECD2
MADSNGTITVEELKKLLEQWNLVIGFLFLTWICLLQFAYANRNRFLYIIKLIFLWLLWPVTLAC
FVLAAVYRINWITGGIAIAMACLVGLMWLSYFIASFRLFARTRSMWSFNPETNILLNVPLHGTI
LTRPLLESELVIGAVILRGHLRIAGHHLGRCDIKDLPKEITVATSRTLSYYKLGASQRVAGDSG
FAAYSRYRIGNYKLNTDHSSSSDNIALLVQ
SEQ ID NO: 46, Rhavi-M fused ECDs
[Rhavi-linker-M protein fused ECDs]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSMADSNGTITVEELKKLLEQWNLVIGFLFLTWICLLQFAYANRNRFLYIIK
LIFLWLLWPVTLACFVLAAVYRINWITGGIAIAMACLVGLMWLSYFIASFRLFARTRSMWSFNP
ETNILLNVPLHGTILTRPLLESELVIGAVILRGHLRIAGHHLGRCDIKDLPKEITVATSRTLSY
YKLGASQRVAGDSGFAAYSRYRIGNYKLNTDHSSSSDNIALLVQ
SEQ ID NO: 47, M fused ECDs-Rhavi
[M protein fused ECDs-linker-Rhavi]
ADSNGTITVEELKKLLEQWNLVIGFLFLTWICLLQFAYANRNRFLYIIKLIFLWLLWPVTLACF
VLAAVYRINWITGGIAIAMACLVGLMWLSYFIASFRLFARTRSMWSFNPETNILLNVPLHGTIL
TRPLLESELVIGAVILRGHLRIAGHHLGRCDIKDLPKEITVATSRTLSYYKLGASQRVAGDSGF
AAYSRYRIGNYKLNTDHSSSSDNIALLVQGGGGSGGGGSGGGGSMFDASNFKDFSSIASASSSW
QNQSGSTMIIQVDSFGAVSGQYVNRAQGTGCQNSPYPLTGRVNGAFIAFSVGWNAATENCNSAT
GWTGYAQVNGANTEIVTSWNLAYEGGSGPAIEQGQDTFQYVPTTENKSLLKD
SEQ ID NO: 48, S-RBD-Rhavi-M fused ECDs
[Spike glycoprotein RBD-linker-Rhavi-linker-M protein fused ECDs]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TVCGPGGGGSGGGGSGGGGSMFDASNFKDFSSIASASSSWQNQSGSTMIIQVDSFGAVSGQYVN
RAQGTGCQNSPYPLTGRVNGAFIAFSVGWNAATENCNSATGWTGYAQVNGANTEIVTSWNLAYE
GGSGPAIEQGQDTFQYVPTTENKSLLKDGGGGSSSMADSNGTITVEELKKLLEQWNLVIGFLFL
TWICLLQFAYANRNRFLYIIKLIFLWLLWPVTLACFVLAAVYRINWITGGIAIAMACLVGLMWL
SYFIASFRLFARTRSMWSFNPETNILLNVPLHGTILTRPLLESELVIGAVILRGHLRIAGHHLG
RCDIKDLPKEITVATSRTLSYYKLGASQRVAGDSGFAAYSRYRIGNYKLNTDHSSSSDNIALLV
Q
SEQ ID NO: 49, M fused ECDs-Rhavi-S-RBD
[M protein fused ECDs-linker-Rhavi-linker-Spike glycoprotein RBD]
ADSNGTITVEELKKLLEQWNLVIGFLFLTWICLLQFAYANRNRFLYIIKLIFLWLLWPVTLACF
VLAAVYRINWITGGIAIAMACLVGLMWLSYFIASFRLFARTRSMWSFNPETNILLNVPLHGTIL
TRPLLESELVIGAVILRGHLRIAGHHLGRCDIKDLPKEITVATSRTLSYYKLGASQRVAGDSGF
AAYSRYRIGNYKLNTDHSSSSDNIALLVQGGGGSGGGGSGGGGSMFDASNFKDFSSIASASSSW
QNQSGSTMIIQVDSFGAVSGQYVNRAQGTGCQNSPYPLTGRVNGAFIAFSVGWNAATENCNSAT
GWTGYAQVNGANTEIVTSWNLAYEGGSGPAIEQGQDTFQYVPTTENKSLLKDGGGGSSSNITNL
CPFGEVFNATRFASVYAWNRKRISNCVADYSVLYNSASFSTFKCYGVSPTKLNDLCFTNVYADS
FVIRGDEVRQIAPGQTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVLSFELLHAPATVCGP
SEQ ID NO: 50, Rhavi-S-RBD-M fused ECDs
[Rhavi-linker-Spike glycoprotein RBD-linker-M protein fused ECDs]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQSYGFQPTNGVGYQPY
RVVVLSFELLHAPATVCGPGGGGSSSMADSNGTITVEELKKLLEQWNLVIGFLFLTWICLLQFA
YANRNRFLYIIKLIFLWLLWPVTLACFVLAAVYRINWITGGIAIAMACLVGLMWLSYFIASFRL
FARTRSMWSFNPETNILLNVPLHGTILTRPLLESELVIGAVILRGHLRIAGHHLGRCDIKDLPK
EITVATSRTLSYYKLGASQRVAGDSGFAAYSRYRIGNYKLNTDHSSSSDNIALLVQ
SEQ ID NO: 51, S-RBD-M fused ECDs-Rhavi
[Spike glycoprotein RBD-linker-M protein fused ECDs-linker-Rhavi]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TVCGPGGGGSSSMADSNGTITVEELKKLLEQWNLVIGFLFLTWICLLQFAYANRNRFLYIIKLI
FLWLLWPVTLACFVLAAVYRINWITGGIAIAMACLVGLMWLSYFIASFRLFARTRSMWSFNPET
NILLNVPLHGTILTRPLLESELVIGAVILRGHLRIAGHHLGRCDIKDLPKEITVATSRTLSYYK
LGASQRVAGDSGFAAYSRYRIGNYKLNTDHSSSSDNIALLVQGGGGSGGGGSGGGGSMFDASNF
KDFSSIASASSSWQNQSGSTMIIQVDSFGAVSGQYVNRAQGTGCQNSPYPLTGRVNGAFIAFSV
GWNAATENCNSATGWTGYAQVNGANTEIVTSWNLAYEGGSGPAIEQGQDTFQYVPTTENKSLLK
D
SEQ ID NO: 52, Rhavi-M fused ECDs-S-RBD
[Rhavi-linker-M protein fused ECDs-linker-Spike glycoprotein RBD]
FDASNFKDFSSIASASSSWQNQSGSTMIIQVDSFGAVSGQYVNRAQGTGCQNSPYPLTGRVNGA
FIAFSVGWNAATENCNSATGWTGYAQVNGANTEIVTSWNLAYEGGSGPAIEQGQDTFQYVPTTE
NKSLLKDGGGGSSSMADSNGTITVEELKKLLEQWNLVIGFLFLTWICLLQFAYANRNRFLYIIK
LIFLWLLWPVTLACFVLAAVYRINWITGGIAIAMACLVGLMWLSYFIASFRLFARTRSMWSFNP
ETNILLNVPLHGTILTRPLLESELVIGAVILRGHLRIAGHHLGRCDIKDLPKEITVATSRTLSY
YKLGASQRVAGDSGFAAYSRYRIGNYKLNTDHSSSSDNIALLVQGGGGSSSNITNLCPFGEVFN
ATRFASVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPATVCGP
SEQ ID NO: 53, M fused ECDs-S-RBD-Rhavi
[M protein fused ECDs-linker-Spike glycoprotein RBD-linker-Rhavi]
ADSNGTITVEELKKLLEQWNLVIGFLFLTWICLLQFAYANRNRFLYIIKLIFLWLLWPVTLACF
VLAAVYRINWITGGIAIAMACLVGLMWLSYFIASFRLFARTRSMWSFNPETNILLNVPLHGTIL
TRPLLESELVIGAVILRGHLRIAGHHLGRCDIKDLPKEITVATSRTLSYYKLGASQRVAGDSGF
AAYSRYRIGNYKLNTDHSSSSDNIALLVQGGGGSSSNITNLCPFGEVFNATRFASVYAWNRKRI
SNCVADYSVLYNSASFSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTEIYQAGSTPCNGVEGF
NCYFPLQSYGFQPTNGVGYQPYRVVVLSFELLHAPATVCGPGGGGSGGGGSGGGGSMFDASNFK
DFSSIASASSSWQNQSGSTMIIQVDSFGAVSGQYVNRAQGTGCQNSPYPLTGRVNGAFIAFSVG
WNAATENCNSATGWTGYAQVNGANTEIVTSWNLAYEGGSGPAIEQGQDTFQYVPTTENKSLLKD
SEQ ID NO: 54, linker sequence [7 amino acids]:
GGGGSSS
linker sequence [3 amino acids ] :
AAA
SEQ ID NO: 56, linker sequence [5 amino acid repeats]:
(GGGGS)n
SEQ ID NO: 57, linker sequence [6 amino acids]:
GGGGGG
SEQ ID NO: 58, linker sequence [15 amino acids]:
GGGGSGGGGSGGGGS
SEQ ID NO: 59, linker sequence [16 amino acids]:
GGGGSGGGGSGGGGSM
SEQ ID NO: 60, linker sequence [30 amino acids]:
GGGGSGGGGSGGGGSGGGGSGGGGSGGGGS
SEQ ID NO: 61, linker sequence [18 amino
KESGSVSSEQLAQFRSLD
SEQ ID NO: 62, linker sequence [14 amino acids]:
EGKSSGSGSESKST
linker sequence:
(Gly)ₙ
SEQ ID NO: 64, linker sequence [8 amino acids]:
GGGGGGGG
SEQ ID NO: 65, linker sequence [12 amino acids]:
GSAGSAAGSGEF
SEQ ID NO: 66, linker sequence [5 amino acid repeats
(EAAAK)ₙ
SEQ ID NO: 67, linker sequence:
A(EAAAK)ₙA
SEQ ID NO: 68, linker sequence:
A(EAAAK)₄ALEA(EAAAK)₄A
SEQ ID NO: 69, linker sequence:
[A(EAAAK)ₙA]ₘ
SEQ ID NO: 70, linker sequence [12 amino acids]:
AEAAAKEAAAKA
linker sequence [2 amino acid repeats]:
(XP)ₙ
linker sequence [2 amino acid repeats]:
(AP)ₙ
linker sequence [2 amino acid repeats]:
(KP)ₙ
linker sequence [2 amino acid repeats]:
(QP)ₙ
SEQ ID NO: 75, linker sequence [14 amino acids]:
APAPAPAPAPAPAP
SEQ ID NO: 76, GAG linker sequence [21 amino acids]:
GAPGGGGGAAAAAGGGGGGAP
SEQ ID NO: 77, GAG2 linker sequence [39 amino acids]:
GAPGGGGGAAAAAGGGGGGAPGGGGGAAAAAGGGGGGAP
SEQ ID NO: 78, GAGSlinker sequence [57 amino acids]:
GAPGGGGGAAAAAGGGGGGAPGGGGGAAAAAGGGGGGAPGGGGGAAAAAGGGGGGAP
SEQ ID NO: 79, Hisx6 tag:
HHHHHH
SEQ ID NO: 80
MFVFLVLLPLVSSQCVNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSKTQSLLIVNNATNVVI
KVCEFQFCNDPFLGVYYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHRSYLTPGDSSS
GWTAGAAAYYVGYLQPRTFLLKYNENGTITDAVDCALDPLSETKCTLKSFTVEKGIYQTSNFRV
QPTESIVRFPNITNLCPFGEVFNATRFASVYAWNRKRISNCVADYSVLYNSASFSTFKCYGVSP
TKLNDLCFTNVYADSFVIRGDEVRQIAPGQTGKIADYNYKLPDDFTGCVIAWNSNNLDSKVGGN
YNYLYRLFRKSNLKPFERDISTEIYQAGSTPCNGVEGFNCYFPLQSYGFQPTNGVGYQPYRVVV
LSFELLHAPATVCGPKKSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAIHADQLTPTWRVYSTGS
NVFQTRAGCLIGAEHVNNSYECDIPIGAGIGASYQTQTNSPRRARSVASQSIIAYTMSLGAENS
VAYSNNSIAIPTNFTISVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQI
PFAMQMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVLKGVKLHYT
SEQ ID NO: 81, surface glycoprotein RBD [Ancestral Wuhan seafood
market pneumonia virus]
NITNLCPFGEVFNATRFASVYAWNRKRISNCVADYSVLYNSASFSTFKCYGVSPTKLNDLCFTN
VYADSFVIRGDEVRQIAPGQTGKIADYNYKLPDDFTGCVIAWNSNNLDSKVGGNYNYLYRLFRK
SNLKPFERDISTEIYQAGSTPCNGVEGFNCYFPLQSYGFQPTNGVGYQPYRVVVLSFELLHAPA
TV
SEQ ID NO: 82, Receptor Binding Motif (RBM) in surface
glycoprotein RBD [Ancestral Wuhan seafood market pneumonia virus]
NSNNLDSKVGGNYNYLYRLFRKSNLKPFERDISTEIYQAGSTPCNGVEGFNCYFPLQSYGFQPT
NGVGYQPY

1. A vaccine comprising one or more species of immunogenic complexes, wherein each immunogenic complex comprises:
(a) a biotinylated polysaccharide antigen; and
(b) a fusion protein comprising:
(i) a biotin-binding moiety; and
(ii) at least one polypeptide antigen of SARS-CoV-2;
wherein in each species of the immunogenic complexes, the biotinylated polysaccharide antigen is non-covalently associated with the biotin-binding moiety of the fusion protein.

2. The vaccine of claim 1, wherein the fusion protein comprises at least one of:
(a) a Spike (S) polypeptide antigen or antigenic fragment thereof;
(b) an Envelope (E) polypeptide antigen or antigenic fragment thereof;
(c) a Membrane (M) polypeptide antigen or antigenic fragment thereof; and
(d) a Nucleocapsid (N) polypeptide or antigenic fragment thereof.

3. The vaccine of claim 2, wherein the fusion protein comprises at least one of:
(a) a Spike (S) polypeptide antigen or antigenic fragment thereof; and
(b) a Membrane (M) polypeptide antigen or antigenic fragment thereof.

4. (canceled)

5. (canceled)

6. The vaccine of claim 3, wherein the fusion protein comprises:
(a) the Receptor Binding Domain (RBD) of a Spike (S) polypeptide antigen or antigenic fragment thereof; and/or
(b) one or more Extra-Cellular Domains (ECDs) of a Membrane (M) polypeptide antigen or antigenic fragment thereof.

7. The vaccine of claim 1, wherein the one or more species of immunogenic complexes comprise polypeptide antigen(s) of one strain (variant) of SARS-CoV-2.

8. The vaccine of claim 1, wherein the one or more species of immunogenic complexes comprise polypeptide antigen(s) of multiple strains (variants) of SARS-CoV-2.

9. The vaccine of claim 1, comprising one species of immunogenic complexes, wherein the species comprises the same fusion protein.

10. The vaccine of claim 1, comprising a plurality of different species of immunogenic complexes, wherein the plurality of different species comprises a plurality of different fusion proteins.

11. The vaccine of claim 1, wherein at least one of the polypeptide antigens is or comprises an amino acid sequence at least 90%, at least 91%, at least 92%, at least 93%, at least 94%, at least 95%, at least 96%, at least 97%, at least 98%, at least 99%, at least 99.5%, or 100% identical to any of SEQ ID NO:3, 6, 9, 12, 15, 18, 21, 24, 27, 30, 33, 36, 39 or 42, or an antigenic fragment thereof.

12. The vaccine of claim 11, wherein the fusion protein is or comprises an amino acid sequence at least 90%, at least 91%, at least 92%, at least 93%, at least 94%, at least 95%, at least 96%, at least 97%, at least 98%, at least 99%, or at least 99.5% identical to SEQ ID NO:5, 8, 11, 14, 17, 20, 23, 26, 29, 32, 35, 38, 41 or 44, or an antigenic fragment thereof.

13. The vaccine of claim 1, wherein the biotinylated polysaccharide antigen comprises a polysaccharide of Streptococcus pneumoniae.

14. The vaccine of claim 13, wherein the biotinylated polysaccharide antigen comprises a polysaccharide of Streptococcus pneumoniae selected from serotypes 1, 9N, and 19A.

15. The vaccine of claim 14, wherein the biotinylated polysaccharide antigen comprises a polysaccharide of Streptococcus pneumoniae serotype 1 (PS1).

16. The vaccine of claim 1, wherein the biotin-binding moiety is a polypeptide comprising (i) an amino acid sequence at least 90%, at least 91%, at least 92%, at least 93%, at least 94%, at least 95%, at least 96%, at least 97%, at least 98%, at least 99%, or at least 99.5%, or 100% identical to SEQ ID NO:1 or a biotin-binding fragment thereof; or (ii) a polypeptide comprising an amino acid sequence at least 90%, at least 91%, at least 92%, at least 93%, at least 94%, at least 95%, at least 96%, at least 97%, at least 98%, at least 99%, or at least 99.5% identical to SEQ ID NO:2, or a biotin-binding fragment thereof.

17. An immunogenic complex comprising:
(a) a biotinylated polysaccharide antigen; and
(b) a fusion protein comprising:
(i) a biotin-binding moiety; and
(ii) at least one polypeptide antigen of SARS-CoV-2;
wherein the biotinylated polysaccharide antigen is non-covalently associated with the biotin-binding moiety of the fusion protein.

18. (canceled)

19. A pharmaceutical composition comprising the vaccine of claim 1, and a pharmaceutically acceptable carrier.

20. (canceled)

21. A method of making the vaccine of claim 1, comprising non-covalently complexing a plurality of biotinylated polysaccharide antigens with a plurality of fusion proteins to form the one or more species of immunogenic complexes, wherein each fusion protein comprises at least one polypeptide antigen of SARS-CoV-2 selected from:
(a) a Spike (S) polypeptide antigen or antigenic fragment thereof;
(b) an Envelope (E) polypeptide antigen or antigenic fragment thereof;
(c) a Membrane (M) polypeptide antigen or antigenic fragment thereof; and
(d) a Nucleocapsid (N) polypeptide or antigenic fragment thereof.

22. A method of immunizing a subject against one or more strains (variants) of SARS-CoV-2 comprising administering to the subject an immunologically effective amount of the vaccine of claim 1.

23. (canceled)

24. (canceled)

25. A fusion protein comprising:
(i) a biotin-binding moiety;
(ii) at least one polypeptide antigen of SARS-CoV-2.

26. A nucleic acid that comprises a nucleotide sequence encoding the fusion protein of claim 25.


SEQUENCE LISTING (88 sequences)

SEQ ID NO: 1 - protein, 179 aa, Rhizobium sp.
MIITSLYATFGTIADGRRTSGGKTMIRTNAVAALVFAVATSALAFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 2 - protein, 135 aa, Rhizobium sp.
FDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

SEQ ID NO: 3 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 4 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 5 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 6 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGP

SEQ ID NO: 7 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGP

SEQ ID NO: 8 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 9 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 10 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 11 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 12 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 13 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 14 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 15 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 16 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGP

SEQ ID NO: 17 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 18 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 19 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGP

SEQ ID NO: 20 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 21 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGP

SEQ ID NO: 22 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGP

SEQ ID NO: 23 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 24 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGP

SEQ ID NO: 25 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGP

SEQ ID NO: 26 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 27 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGP

SEQ ID NO: 28 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGP

SEQ ID NO: 29 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 30 - protein, 197 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGP

SEQ ID NO: 31 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGP

SEQ ID NO: 32 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 33 - protein, 197 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 34 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 35 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 36 - protein, 197 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 37 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 38 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 39 - protein, 197 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 40 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGP

SEQ ID NO: 41 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 42 - protein, 197 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 43 - protein, 339 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGP

SEQ ID NO: 44 - protein, 348 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 45 - protein, 222 aa, synthetic construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 46 - protein, 364 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 47 - protein, 372 aa, synthetic construct
ADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 48 - protein, 577 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 49 - protein, 576 aa, synthetic construct
ADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 50 - protein, 568 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSSSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 51 - protein, 577 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSSSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 52 - protein, 568 aa, synthetic construct
FDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKDGGGGSSSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 53 - protein, 576 aa, synthetic construct
ADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGGGGSSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSMFDASNFKDFSSIASASSSWQNQSGSTMIIQVDSFGAVSGQYVNRAQGTGCQNSPYPLTGRVNGAFIAFSVGWNAATENCNSATGWTGYAQVNGANTEIVTSWNLAYEGGSGPAIEQGQDTFQYVPTTENKSLLKD

SEQ ID NO: 54 - protein, 7 aa, synthetic construct
GGGGSSS

SEQ ID NO: 55 - skipped

SEQ ID NO: 56 - protein, 5 aa, synthetic construct
GGGGS

SEQ ID NO: 57 - protein, 6 aa, synthetic construct
GGGGGG

SEQ ID NO: 58 - protein, 15 aa, synthetic construct
GGGGSGGGGSGGGGS

SEQ ID NO: 59 - protein, 16 aa, synthetic construct
GGGGSGGGGSGGGGSM

SEQ ID NO: 60 - protein, 30 aa, synthetic construct
GGGGSGGGGSGGGGSGGGGSGGGGSGGGGS

SEQ ID NO: 61 - protein, 18 aa, synthetic construct
KESGSVSSEQLAQFRSLD

SEQ ID NO: 62 - protein, 14 aa, synthetic construct
EGKSSGSGSESKST

SEQ ID NO: 63 - skipped

SEQ ID NO: 64 - protein, 8 aa, synthetic construct
GGGGGGGG

SEQ ID NO: 65 - protein, 12 aa, synthetic construct
GSAGSAAGSGEF

SEQ ID NO: 66 - protein, 5 aa, synthetic construct
EAAAK

SEQ ID NO: 67 - protein, 7 aa, synthetic construct
AEAAAKA

SEQ ID NO: 68 - protein, 46 aa, synthetic construct
AEAAAKEAAAKEAAAKEAAAKALEAEAAAKEAAAKEAAAKEAAAKA

SEQ ID NO: 69 - protein, 7 aa, synthetic construct
AEAAAKA

SEQ ID NO: 70 - protein, 12 aa, synthetic construct
AEAAAKEAAAKA

SEQ ID NO: 71-74 - skipped (4 entries)

SEQ ID NO: 75 - protein, 14 aa, synthetic construct
APAPAPAPAPAPAP

SEQ ID NO: 76 - protein, 21 aa, synthetic construct
GAPGGGGGAAAAAGGGGGGAP

SEQ ID NO: 77 - protein, 39 aa, synthetic construct
GAPGGGGGAAAAAGGGGGGAPGGGGGAAAAAGGGGGGAP

SEQ ID NO: 78 - protein, 57 aa, synthetic construct
GAPGGGGGAAAAAGGGGGGAPGGGGGAAAAAGGGGGGAPGGGGGAAAAAGGGGGGAP

SEQ ID NO: 79 - protein, 6 aa, synthetic construct
HHHHHH

SEQ ID NO: 80 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 81 - protein, 194 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 82 - protein, 72 aa, Severe acute respiratory syndrome coronavirus 2
NSNNLDSKVGGNYNYLYRLFRKSNLKPFERDISTEIYQAGSTPCNGVEGFNCYFPLQSYGFQPTNGVGYQPY

SEQ ID NO: 83 - DNA, 20 bp, synthetic construct
  source 1..20: mol_type = other DNA
gaccccaaaatcagcgaaat

SEQ ID NO: 84 - DNA, 24 bp, synthetic construct
  source 1..24: mol_type = other DNA
tctggttactgccagttgaatctg

SEQ ID NO: 85 - DNA, 24 bp, synthetic construct
  source 1..24: mol_type = other DNA
  modified_base 1: mod_base = OTHER; note = Fluorescein amidite adenine
  modified_base 24: mod_base = OTHER; note = Cytosine Black hole quencher 1
accccgcattacgtttggtggacc

SEQ ID NO: 86 - DNA, 30 bp, synthetic construct
  source 1..30: mol_type = other DNA
cgatcttgtagatctgttcctcaaacgaac

SEQ ID NO: 87 - DNA, 24 bp, synthetic construct
  source 1..24: mol_type = other DNA
atattgcagcagtacgcacacaca

SEQ ID NO: 88 - DNA, 26 bp, synthetic construct
  source 1..26: mol_type = other DNA
  modified_base 1: mod_base = OTHER; note = Fluorescein amidite adenine
  modified_base 26: mod_base = OTHER; note = Guanine Black hole quencher
acactagccatccttactgcgcttcg